(19) United States
(12) Patent Application Publication
(10) Pub. No.: US 2010/0105722 A1
(43) Pub. Date: Apr. 29, 2010

(54) Substituted 4,5,6,7-tetrahydrothienopyridines as KCNQ2/3 Modulators

(75) Inventors: Sven Kuehnert, Dueren (DE); Gregor Bahrenberg, Monschau-Konzen (DE); Achim Kless, Aachen (DE); Beatrix Merla, Aachen (DE); Klaus Schiene, Juechen (DE); Wolfgang Schroeder, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: GRUENENTHAL GmbH, Aachen (DE)

(21) Appl. No.: 12/604,534

(22) Filed: Oct. 23, 2009

Related U.S. Application Data
(60) Provisional application No. 61/108,180, filed on Oct. 24, 2008.

(30) Foreign Application Priority Data
Oct. 24, 2008 (EP) 08018617.4

(51) Int. Cl. A61K 31/4365; C07D 495/04; A61P 25/00; A61P 25/22; A61P 25/06

(52) U.S. Cl. 514/301; 546/114

(57) ABSTRACT

Substituted tetrahydrothienopyridines corresponding to formula (1)

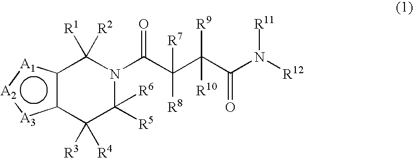

in which A₁ through A₃ and R¹ through R¹² have defined meanings, pharmaceutical compositions comprising such compounds, a process for preparing such compounds and the use of such compounds in treatment or inhibition of conditions mediated by the KCNQ 2/3 K⁺ channel, e.g., pain.

### BACKGROUND OF THE INVENTION

The invention relates to substituted tetrahydrothienopyridines, to processes for their preparation, to medicaments comprising these compounds and to the use of these compounds in the preparation of medicaments.

The treatment of pain, in particular of neuropathic pain, is of great importance in medicine. There is a worldwide need for effective pain therapies. The urgent need for action for a target-orientated treatment of chronic and non-chronic states of pain appropriate for the patient, by which is to be understood the successful and satisfactory treatment of pain for the patient, is also documented in the large number of scientific works which have recently been published in the field of applied analgesics and of fundamental research into nociception.

A pathophysiological feature of chronic pain is the overexcitability of neurons. Neuronal excitability is influenced decisively by the activity of K⁺ channels, since these determine decisively the resting membrane potential of the cell and therefore the excitability threshold. Heteromeric K⁺ channels of the molecular subtype KCNQ2/3 (Kv7.2/7.3) are expressed in neurons of various regions of the central (hippocampus, amygdala) and peripheral (dorsal root ganglia) nervous system and regulate the excitability thereof. Activation of KCNQ2/3 K⁺ channels leads to a hyperpolarization of the cell membrane and, accompanying this, to a decrease in the electrical excitability of these neurons. KCNQ2/3-expressing neurons of the dorsal root ganglia are involved in the transmission of nociceptive stimuli from the periphery into the spinal marrow (Passmore et al., J. Neurosci. 2003; 23(18): 7227-36).

It has accordingly been possible to detect an analgesic activity in preclinical neuropathy and inflammatory pain models for the KCNQ2/3 agonist retigabine (Blackburn-Munro and Jensen, Eur J Pharmacol. 2003; 460(2-3); 109-16; Dost et al., Naunyn Schmiedebergs Arch Pharmacol 2004; 369(4): 382-390).

The KCNQ2/3 K⁺ channel thus represents a suitable starting point for the treatment of pain; in particular of pain selected from the group consisting of chronic pain, neuropathic pain, inflammatory pain and muscular pain (Nielsen et al., Eur J Pharmacol. 2004; 487(1-3): 93-103), especially neuropathic or inflammatory pain.

Moreover, the KCNQ2/3 K⁺ channel is a suitable target for therapy of a large number of further diseases, such as, for example, migraine (US2002/0128277), cognitive diseases (Gribkoff, Expert Opin Ther Targets 2003; 7(6): 737-748), anxiety (Korsgaard et al., J Pharmacol Exp Ther. 2005, 14(1): 282-92), epilepsy (Wickenden et al., Expert Opin Ther Pat 2004; 14(4): 457-469; Gribkoff, Expert Opin Ther Targets 2008, 12(5): 565-81; Miceli et al., Curr Opin Pharmacol 2008, 8(1): 65-74), urinary incontinence (Streng et al., J Urol 2004; 172: 2054-2058), dependency (Hansen et al., Eur J Pharmacol 2007, 570(1-3): 77-88), mania/bipolar disorders (Dencker et al., Epilepsy Behav 2008, 12(1): 49-53), dystonia-associated dyskinesias (Richter et al., Br J Pharmacol 2006, 149(6): 747-53).

### SUMMARY OF THE INVENTION

It was therefore an object of the present invention to provide novel compounds which have advantages over the compounds of the prior art.

Another object of the invention was to provide compounds which are suitable in particular as pharmacological active ingredients in pharmaceutical compositions.

A further object of the invention was to provide compounds which are useful in the treatment of disorders or diseases that are mediated at least in part by KCNQ2/3 K⁺ channels.

Another object of the invention was to provide a new method of treating o inhibiting pain.

These and other objects have been achieved by the invention as described and claimed hereinafter.

It has been found, surprisingly, that substituted tetrahydrothienopyridines of the general formula (1) given below are suitable for the treatment of pain. It has also been found, surprisingly, that substituted tetrahydrothienopyridines of formula (1) given below also have an excellent affinity for the KCNQ2/3 K⁺ channel and are therefore suitable for the treatment of disorders or diseases that are mediated at least in part by KCNQ2/3 K⁺ channels. The substituted tetrahydrothienopyridines thereby act as modulators, that is to say agonists or antagonists, of the KCNQ2/3 K⁺ channel.

Substituted tetrahydropyrrolopyrazines that have an affinity for the KCNQ2/3 K⁺ channel are known from the prior art (WO 2008/046582).

Substituted tetrahydrothienopyridines and their use in medicaments are described in WO 96/34870. Further tetrahydrothienopyridines are also known, but the use thereof in medicaments is not described (e.g. CA 940806-85-9; CA 931614-62-9; CA 930990-23-1).

The invention provides substituted tetrahydrothienopyridines corresponding to formula (1)

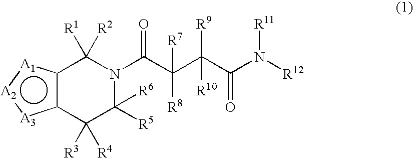

wherein
   A₁ represents S, A₂ represents CR¹⁴ and A₃ represents CR¹⁵; or
   A₁ represents CR¹³, A₂ represents S and A₃ represents CR¹⁵; or
   A₁ represents CR¹³, A₂ represents CR¹⁴ and A₃ represents S;
R⁰ represents C₁₋₈-alkyl, saturated or unsaturated, branched or unbranched, unsubstituted or mono- or poly-substituted; C₃₋₇-cycloalkyl or heterocyclyl, in each case saturated or unsaturated, unsubstituted or mono- or poly-substituted; aryl or heteroaryl, in each case unsubstituted or mono- or poly-substituted; C₁₋₈-alkyl-bridged C₃₋₇-cycloalkyl or heterocyclyl, in each case saturated or unsaturated, unsubstituted or mono- or poly-substituted, wherein the alkyl chain in each case can be branched or unbranched, saturated or unsaturated, unsubstituted, mono- or poly-substituted; or C₁₋₈-alkyl-bridged aryl or heteroaryl, in each case unsubstituted or mono- or poly-substituted, wherein the alkyl chain in each case can be branched or unbranched, saturated or unsaturated, unsubstituted, mono- or poly-substituted;
R¹ represents H; F; Cl; Br; CN; or R⁰;
R² represents H, F, Cl, Br or C₁₋₈-alkyl, saturated or unsaturated, branched or unbranched, unsubstituted or mono- or poly-substituted; or
R¹ and R², together with the carbon atom joining them as ring member, form a C₃₋₇-cycloalkyl or heterocyclyl, in each case saturated or unsaturated, unsubstituted or mono- or poly-substituted, in each case optionally fused with (hetero)aryl, unsubstituted or mono- or poly-substituted;
R³, R⁴, R⁵, R⁶ each independently represent H; F; Cl; Br; C₁₋₈-alkyl, saturated or unsaturated, branched or unbranched, unsubstituted or mono- or poly-substituted; or phenyl, unsubstituted or mono- or poly-substituted;
R⁷ represents H; F; Cl; Br; CN; R⁰; OR⁰; O—C(═O)—R⁰; O—C(═O)—O—R⁰; O—(C═O)—NH—R⁰; O—C(═O)—N(R⁰)₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; C(═O)—OH; C(═O)—OR⁰; C(═O)—NH₂; C(═O)—NH—R⁰; C(═O)—N(R⁰)₂; S(═O)₂—OH; S(═O)₂OR⁰; S(═O)₂—NH₂; S(═O)₂—NH—R⁰; S(═O)₂—N(R⁰)₂;
R⁸ represents H; F; Cl; Br; CN; or C₁₋₈-alkyl, saturated or unsaturated, branched or unbranched, unsubstituted or mono- or poly-substituted;
R⁹ represents H; F; Cl; Br; CN; C₁₋₈-alkyl, saturated or unsaturated, branched or unbranched, unsubstituted or mono- or poly-substituted; C₃₋₇-cycloalkyl or heterocyclyl, in each case saturated or unsaturated, unsubstituted or mono- or poly-substituted; aryl or heteroaryl, in each case unsubstituted or mono- or poly-substituted; C₁₋₈-alkyl-bridged C₃₋₇-cycloalkyl or heterocyclyl, in each case saturated or unsaturated, unsubstituted or mono- or poly-substituted, wherein the alkyl chain in each case can be branched or unbranched, saturated or unsaturated, unsubstituted, mono- or poly-substituted; or C₂₋₈-alkyl-bridged aryl or heteroaryl, in each case unsubstituted or mono- or poly-substituted, wherein the alkyl chain in each case can be branched or unbranched, saturated or unsaturated, unsubstituted, mono- or poly-substituted; O—C₁₋₈-alkyl, saturated or unsaturated, branched or unbranched, unsubstituted or mono- or poly-substituted; O—C₃₋₇-cycloalkyl or O-heterocyclyl, in each case saturated or unsaturated, unsubstituted or mono- or poly-substituted; O—C₁₋₈-alkyl-bridged C₃₋₇-cycloalkyl or heterocyclyl, in each case saturated or unsaturated, unsubstituted or mono- or poly-substituted, wherein the alkyl chain in each case can be branched or unbranched, saturated or unsaturated, unsubstituted, mono- or poly-substituted; or O—C₁₋₈-alkyl-bridged aryl or heteroaryl, in each case unsubstituted or mono- or poly-substituted, wherein the alkyl chain in each case can be branched or unbranched, saturated or unsaturated, unsubstituted, mono- or poly-substituted; O—C(═O—)—R⁰; O—C(═O)—O—R⁰; O—(C═O)—NH—R⁰; O—C(═O)—N(R⁰)₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; C(═O)—OH; C(═O)—OR⁰; C(═O)—NH₂; C(═O)—NH—R⁰; C(═O)—N(R⁰)₂; S(═O)₂—OH; S(═O)₂OR⁰; S(═O)₂—NH₂; S(═O)₂—NH—R⁰; S(═O)₂—N(R⁰)₂;
R¹⁰ represents H; F; Cl; Br; CN; or C₁₋₈-alkyl, saturated or unsaturated, branched or unbranched, unsubstituted or mono- or poly-substituted; or
R⁷ and R⁹, together with the carbon atoms joining them as ring members, form a C₃₋₇-cycloalkyl or heterocyclyl, in each case saturated or unsaturated, unsubstituted or mono- or poly-substituted, optionally fused with (hetero)aryl, unsubstituted or mono- or poly-substituted; or
R⁷ and R⁸, or R⁹ and R¹⁰, together with the carbon atoms joining them as ring members, form a C₃₋₇-cycloalkyl or heterocyclyl, in each case saturated or unsaturated, unsubstituted or mono- or poly-substituted, in each case optionally fused with (hetero)aryl, unsubstituted or mono- or poly-substituted;
R¹¹ represents H; C₁₋₈-alkyl, saturated or unsaturated, branched or unbranched, unsubstituted or mono- or poly-substituted; or C₃₋₇-cycloalkyl, saturated or unsaturated, unsubstituted or mono- or poly-substituted;
R¹² represents C₂₋₁₆-alkyl, saturated or unsaturated; branched or unbranched, unsubstituted or mono- or poly-substituted; C₃₋₇-cycloalkyl or heterocyclyl, in each case saturated or unsaturated, unsubstituted or mono- or poly-substituted; aryl or heteroaryl, unsubstituted or mono- or poly-substituted; C₁₋₈-alkyl-bridged C₃₋₇-cycloalkyl or heterocyclyl, in each case saturated or unsaturated, unsubstituted or mono- or poly-substituted, wherein the alkyl chain in each case can be branched or unbranched, saturated or unsaturated, unsubstituted, mono- or poly-substituted; C₁₋₈-alkyl-bridged aryl or heteroaryl, in each case unsubstituted or mono- or poly-substituted; wherein the alkyl chain in each case can be branched or unbranched, saturated or unsaturated, unsubstituted, mono- or poly-substituted; or
R¹¹ and R¹², together with the nitrogen atom joining them as ring member, form a heterocyclyl, saturated or unsaturated, unsubstituted or mono- or poly-substituted, optionally fused with (hetero)aryl, unsubstituted or mono- or poly-substituted;
R¹³, R¹⁴ and R¹⁵ each independently of the others denotes H; F; Cl; Br; I; NO; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; O—(C₁₋₈-alkyl)-O; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
wherein

“alkyl substituted”, “heterocyclyl substituted” and “cycloalkyl substituted” denote the replacement of one or more hydrogen atoms, in each case each independently, by F; Cl; Br; I; CN; CF₃; ═O; ═NH; ═C(NH₂)₂; NO₂; R⁰; C(═O)H; C(═O)R⁰; CO₂H; C(═O)OR⁰; CONH₂; C(═O)NHR⁰; C(═O)N(R⁰)₂; OH; OR⁰; O—(C₁₋₈-alkyl)-O; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; S(═O)₂N(R⁰)₂; and

“aryl substituted” and “heteroaryl substituted” denote the substitution of one or more hydrogen atoms, in each case each independently, by F; Cl; Br; I; NO; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; O—(C₁₋₈-alkyl)-O; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; S(═O)₂N(R⁰)₂;

with the exception of the following compounds:
1-morpholino-4-(4-(thiophen-2-yl)-6,7-dihydrothieno[3,2-c]pyridin-5(4H)-yl)-butane-1,4-dione,
1-(4-acetylpiperazin-1-yl)-4-(4-(thiophen-2-yl)-6,7-dihydrothieno[3,2-c]pyridin-5(4H)-yl)butane-1,4-dione and
1-(3-phenyl-4,5-dihydropyrazol-1-yl)-4-(4-(thiophen-2-yl)-6,7-dihydrothieno-[3,2-c]pyridin-5(4H)-yl)butane-1,4-dione;
   in the form of the free compounds or of salts of physiologically acceptable acids or bases.

According to the general formula (1), the five-membered aromatic ring fused with the tetrahydropyridine ring always contains a sulfur atom and accordingly represents a thienyl group which is disubstituted by the substituents R¹³ and/or R¹⁴ and/or R¹⁵, each of which can optionally denote H.

Within the scope of this invention the terms “C₁₋₂-alkyl”, “C₁₋₈-alkyl”, “C₁₋₄-alkyl”, “C₂₋₈-alkyl”, “C₂₋₁₆-alkyl”, “C₄₋₁₆-alkyl” include acyclic saturated or unsaturated hydrocarbon radicals, which can be branched or unbranched as well as unsubstituted or mono- or poly-substituted, having from 1 to 2 or from 1 to 4 or from 1 to 8 or from 2 to 8 or from 2 to 16 or from 4 to 16 carbon atoms, respectively, that is to say C₁₋₂-alkanyls and C₁₋₂-alkenyls or C₁₋₄-alkanyls, C₁₋₄-alkenyls and C₂₋₄-alkynyls or C₁₋₈-alkanyls, C₁₋₈-alkenyls and C₂₋₈-alkynyls or C₂₋₈-alkanyls, C₂₋₈-alkenyls and C₂₋₈-alkynyls or C₂₋₁₆-alkanyls, C₂₋₁₆-alkenyls and C₂₋₁₆-alkynyls or C₄₋₁₆-alkanyls, C₄₋₁₆-alkenyls and C₄₋₁₆-alkynyls. Alkenyls contain at least one C—C double bond and alkynyls contain at least one C—C triple bond. Alkyl is preferably selected from the group comprising methyl, ethyl, n-propyl, 2-propyl, n-butyl, isobutyl, sec-butyl, tert-butyl, n-pentyl, isopentyl, neopentyl, n-hexyl, n-heptyl, n-octyl, n-nonyl, n-decyl, n-undecyl, n-dodecyl, n-tridecyl, n-tetradecyl, n-pentadecyl, n-hexadecyl, ethylenyl (vinyl), ethynyl, propenyl (—CH₂CH═CH₂, —CH═CH—CH₃, —C(═CH₂)—CH₃), propynyl (—CH—C≡CH, —C≡C—CH₃), butenyl, butynyl, pentenyl, pentynyl, hexenyl and hexynyl, heptenyl, heptynyl, octenyl, octynyl, nonenyl, nonynyl, decenyl, decynyl, undecenyl, undecynyl, dodecenyl, dodecynyl, tridecenyl, tridecynyl, tetradecenyl, tetradecynyl, pentadecenyl, pentadecynyl, hexadecenyl and hexadecynyl.

For the purposes of this invention the expression “cycloalkyl” or “C₃₋₇-cyclo-alkyl” denotes cyclic hydrocarbons having 3, 4, 5, 6 or 7 carbon atoms, wherein the hydrocarbons can be saturated or unsaturated (but not aromatic), unsubstituted or mono- or poly-substituted. The cycloalkyl can be bonded to the general structure of higher order via any desired and possible ring member of the cycloalkyl radical. The cycloalkyl radicals can also be fused with further saturated, (partially) unsaturated, heterocyclic, aromatic or heteroaromatic ring systems, which in turn can be unsubstituted or mono- or poly-substituted. The cycloalkyl radicals can further be bridged one or more times, as in the case of adamantyl or dicyclopentadienyl, for example. C₃₋₇-Cycloalkyl is preferably selected from the group containing cyclopropyl, cyclobutyl, cyclopentyl, cyclohexyl, cycloheptyl, cyclopentenyl, cyclohexenyl and cycloheptenyl.

The term “heterocyclyl” includes saturated or unsaturated (but not aromatic) cycloalkyls having from three to seven ring members, in which one, two or three carbon atoms have been replaced by a hetero atom in each case selected each independently from the group S, N and O, it being possible for the ring members to be unsubstituted or mono- or poly-substituted. The heterocyclyl can be bonded to the general structure of higher order via any desired and possible ring member of the heterocyclyl radical. The heterocyclyl radicals can also be fused with further saturated, (partially) unsaturated or aromatic or heteroaromatic ring systems, which in turn can be unsubstituted or mono- or poly-substituted. Heterocyclyl radicals from the group azetidinyl, aziridinyl, azepanyl, quinolinyl, dioxanyl, dioxolanyl, furanyl, imidazolidinyl, isoxazolidinyl, isoquinolinyl, indolinyl, morpholinyl, pyranyl, pyrrolyl, pyridinyl, pyrrolyl, pyrrolidinyl, piperazinyl, piperidinyl, pyrazolidinyl, pyrazolinonyl and thiomorpholinyl are preferred. Heterocyclyl radicals from the group morpholinyl, piperazinyl and piperidinyl are particularly preferred.

Within the scope of this invention the term “aryl” denotes aromatic hydrocarbons having up to 14 ring members, inter alia phenyls and naphthyls. Each aryl radical can be unsubstituted or mono- or poly-substituted, it being possible for the aryl substituents to be identical or different and to be in any desired and possible position of the aryl. The aryl can be bonded to the general structure of higher order via any desired and possible ring member of the aryl radical. The aryl radicals can also be fused with further saturated, (partially) unsaturated, heterocyclic, aromatic or heteroaromatic ring systems, which in turn can be unsubstituted or mono- or poly-substituted. Aryl is preferably selected from the group containing phenyl, 1-naphthyl and 2-naphthyl, each of which can be unsubstituted or mono- or poly-substituted. A particularly preferred aryl is phenyl, unsubstituted or mono- or poly-substituted.

The term “heteroaryl” represents a 5-, 6- or 7-membered cyclic aromatic radical which contains at least 1 heteroatom, optionally also 2, 3, 4 or 5 heteroatoms, wherein the heteroatoms are in each case selected independently of the others from the group S, N and O and the heteroaryl radical can be unsubstituted or mono- or poly-substituted; in the case of substitution on the heteroaryl, the substituents can be identical or different and can be in any desired and possible position of the heteroaryl. Preferred heteroatoms are S, N and O. S and N are particularly preferred. Bonding to the general structure of higher order can take place via any desired and possible ring member of the heteroaryl radical. The heteroaryl can also be part of a bi- or poly-cyclic system having up to 14 ring members, wherein the ring system can be formed with further saturated, (partially) unsaturated, heterocyclic or aromatic or heteroaromatic rings, which in turn can be unsubstituted or mono- or poly-substituted. Within the scope of this invention, the thienyl ring of formula (1) fused with the tetrahydropyridine ring accordingly represents a heteroaryl and any substituents present are accordingly preferably defined as the substituents of heteroaryl defined hereinbefore. It is preferable for the heteroaryl radical to be selected from the group comprising benzofuranyl, benzimidazolyl, benzothienyl, benzothiadiazolyl, benzothiazolyl, benzotriazolyl, benzodioxolanyl, benzodioxanyl, quinazolinyl, carbazolyl, quinolinyl, furyl (furanyl), imidazolyl, indazolyl, indolizinyl, isoquinolinyl, isoxazolyl, isothiazolyl, indolyl, oxadiazolyl, phthalazinyl, pyrazolyl, pyridyl, pyrrolyl, pyridazinyl, pyrimidinyl, pyrazinyl, purinyl, phenazinyl, thienyl, triazolyl, thiazolyl, thiadiazolyl and triazinyl. Pyridyl and thienyl are particularly preferred.

Within the scope of the invention, the terms “C₁₋₂-alkyl- or C₁₋₄-alkyl- or C₁₋₈-alkyl- or C₂₋₈-alkyl-bridged aryl, heteroaryl, heterocyclyl or cycloalkyl” mean that C₁₋₂-alkyl or C₁₋₄-alkyl or C₁₋₈-alkyl or C₂₋₈-alkyl and aryl or heteroaryl or heterocyclyl or cycloalkyl have the meanings defined above and the aryl or heteroaryl or heterocyclyl or cycloalkyl radical is bonded to the structure of higher order via a C₁₋₂-alkyl or C₁₋₄-alkyl or C₁₋₈-alkyl or C₂₋₈-alkyl group. The alkyl chain can in all cases be saturated or unsaturated, branched or unbranched, unsubstituted or mono- or poly-substituted.

In connection with “alkyl”, “heterocyclyl” and “cycloalkyl”, the expression “mono- or poly-substituted” is understood as meaning within the scope of this invention the substitution of one or more hydrogen atoms, in each case each independently, one or more times, for example two, three or four times, by substituents selected from the group comprising F; Cl; Br; I; CN; CF₃; ═O; ═NH; ═C(NH₂)₂; NO₂; R⁰; C(═O)H; C(═O)R⁰; CO₂H; C(═O)OR⁰; CONH₂; C(═O)NHR⁰; C(═O)N(R⁰)₂; OH; OR⁰; O—(O₁₋₈-alkyl)—O; O—C(═O)—R⁰; O—C(═O)—O—R⁰; O—(C═O)—NH—R⁰; O—O(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; S(═O)₂N(R⁰)₂; wherein polysubstituted radicals are to be understood as being radicals that are substituted several times, for example two, three or four times, either on different atoms or on the same atom, for example three times on the same carbon atom, as in the case of CF₃ or CH₂CF₃, or at different places, as in the case of CH(OH)—CH═CH—CHCl₂. A substituent can itself optionally be mono- or poly-substituted. Polysubstitution can take place with the same substituent or with different substituents.

Preferred “alkyl”, “heterocyclyl” and “cycloalkyl” substituents are F; Cl; Br; I; CN; ═O; ═NH; ═C(NH₂)₂; NO₂; benzyl; C₁₋₈-alkyl; CF₃; C(═O)H; C(═O)OH; C(═O)C₁₋₈-alkyl; C(═O)aryl; C(═O)heteroaryl; C(═O)O—O₁₋₈-alkyl; C(═O)O-aryl; C(═O)O-heteroaryl; C(═O)NH₂; C(═O)NH—O₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(aryl)(heteroaryl); OH; O—C₁₋₈-alkyl; O—C₁₋₈-alkyl-OH; O—(C₁₋₈-alkyl)-O; O—(C₁₋₈-alkyl)—O—C₁₋₈-alkyl; OCF₃; O-aryl; O-heteroaryl; O-benzyl; O—C(═O)—C₁₋₈-alkyl; O—C(═O)-aryl; O—C(═O)-heteroaryl; O—S(═O)₂OH; O—S(═O)₂—OC₁₋₈-alkyl; O—S(═O)₂—O-aryl; O—S(═O)₂—O-heteroaryl; O—S(═O)₂C₁₋₈-alkyl; O—S(═O)₂aryl, O—S(═O)₂heteroaryl; O—S(═O)₂NH₂; O—S(═O)₂NH—C₁₋₈-alkyl; O—S(═O)₂N(C₁₋₈-alkyl)₂; O—S(═O)₂NH-aryl; O—S(═O)₂N(aryl)₂; O—S(═O)₂NH-heteroaryl; O—S(═O)₂N(heteroaryl)₂; O—S(═O)₂N(C₁₋₈-alkyl)(aryl); O—S(═O)₂N(heteroaryl)(aryl); O—S(═O)₂N(C₁₋₈-alkyl)(heteroaryl); NH₂; NH(OH); N═C(NH₂)₂; NH—C₁₋₈-alkyl; NH—C₁₋₈-alkyl-OH; N(C₁₋₈-alkyl)₂; N(C₁₋₈-alkyl-OH)₂; NH-aryl; N(aryl)₂; NH-heteroaryl; N(heteroaryl)₂; N(C₁₋₈-alkyl)(aryl); N(C₁₋₈-alkyl)(heteroaryl); N(aryl)(heteroaryl); NH—C(═O)C₁₋₈-alkyl; NH—C(═O)-aryl; NH—C(═O)-heteroaryl; N(C₁₋₈-alkyl)-C(═O)C₁₋₈-alkyl; N(C₁₋₈-alkyl)-C(═O)aryl; N(C₁₋₈-alkyl)-C(═O)heteroaryl; N(aryl)-C(═O)C₁₋₈-alkyl; N(aryl)-C(═O)aryl; N(aryl)-C(═O)heteroaryl; N(heteroaryl)-C(═O)C₁₋₈-alkyl; N(heteroaryl)-C(═O)aryl; N(heteroaryl)-C(═O)heteroaryl; NH—S(═O)₂OH; NH—S(═O)₂C₁₋₈-alkyl; NH—S(═O)₂aryl; NH—S(═O)₂heteroaryl; NH—S(═O)₂OC₁₋₈-alkyl; NH—S(═O)₂O-aryl; NH—S(═O)₂O-heteroaryl; NH—S(═O)₂NH₂; NH—S(═O)₂NH(C₁₋₈-alkyl); NH—S(═O)₂NH(aryl); NH—S(═O)₂NH(heteroaryl); NH—S(═O)₂N(C₁₋₈-alkyl)₂; NH—S(═O)₂N(aryl)₂; NH—S(═O)₂N(heteroaryl)₂; NH—S(═O)₂N(C₁₋₈-alkyl)(aryl); NH—S(═O)₂N(C₁₋₈-alkyl)(heteroaryl); NH—S(═O)₂N(aryl)(heteroaryl); N(C₁₋₈-alkyl)-S(═O)₂OH; N(aryl)-S(═O)₂OH; N(heteroaryl)-S(═O)₂OH; N(C₁₋₈-alkyl)-S(═O)₂—C₁₋₈-alkyl; N(C₁₋₈-alkyl)-S(═O)₂-aryl; N(C₁₋₈-alkyl)-S(═O)₂-heteroaryl; N(aryl)-S(═O)₂—C₁₋₈-alkyl; N(aryl)-S(═O)₂-aryl; N(aryl)-S(═O)₂-heteroaryl; N(heteroaryl)-S(═O)₂—C₁₋₈-alkyl; N(heteroaryl)-S(═O)₂-aryl; N(heteroaryl)-S(═O)₂-heteroaryl; N(C₁₋₈-alkyl)-S(═O)₂—OC₁₋₈-alkyl; N(C₁₋₈-alkyl)-S(═O)₂—O-aryl; N(C₁₋₈-alkyl)-S(═O)₂—O-heteroaryl; N(aryl)-S(═O)₂—OC₁₋₈-alkyl; N(aryl)-S(═O)₂—O-aryl; N(aryl)-S(═O)₂—O-heteroaryl; N(heteroaryl)-S(═O)₂—OC₁₋₈-alkyl; N(heteroaryl)-S(═O)₂—O-aryl; N(heteroaryl)-S(═O)₂—O-heteroaryl; N(C₁₋₈-alkyl)-S(═O)₂NH₂; N(aryl)-S(═O)₂NH₂; N(heteroaryl)-S(═O)₂NH₂; N(C₁₋₈-alkyl)-S(═O)₂NH(C₁₋₈-alkyl); N(C₁₋₈-alkyl)-S(═O)₂NH(aryl); N(C₁₋₈-alkyl)-S(═O)₂NH(heteroaryl); N(C₁₋₈-alkyl)-S(═O)₂N(C₁₋₈-alkyl)₂; N(C₁₋₈-alkyl)-S(═O)₂N(aryl)₂; N(C₁₋₈-alkyl)-S(═O)₂N(heteroaryl)₂; N(C₁₋₈-alkyl)-S(═O)₂N(C₁₋₈-alkyl)(aryl); N(C₁₋₈-alkyl)-S(═O)₂N(C₁₋₈-alkyl)(heteroaryl); N(C₁₋₈-alkyl)-S(═O)₂N(aryl)(heteroaryl); N(aryl)-S(═O)₂NH(C₁₋₈-alkyl); N(aryl)-S(═O)₂NH(aryl); N(aryl)-S(═O)₂NH(heteroaryl); N(aryl)-S(═O)₂N(C₁₋₈-alkyl)₂; N(aryl)-S(═O)₂N(aryl)₂; N(aryl)-S(═O)₂N(heteroaryl)₂; N(aryl)-S(═O)₂N(C₁₋₈-alkyl)(aryl); N(aryl)-S(═O)₂N(C₁₋₈-alkyl)(heteroaryl); N(aryl)-S(═O)₂N(aryl)(heteroaryl); N(heteroaryl)-S(═O)₂NH(C₁₋₈-alkyl); N(heteroaryl)-S(═O)₂NH(aryl); N(heteroaryl)-S(═O)₂NH(heteroaryl); N(heteroaryl)-S(═O)₂N(C₁₋₈-alkyl)₂; N(heteroaryl)-S(═O)₂N(aryl)₂; N(heteroaryl)-S(═O)₂N(heteroaryl)₂; N(heteroaryl)-S(═O)₂N(C₁₋₈-alkyl)(aryl); N(heteroaryl)-S(═O)₂N(C₁₋₈-alkyl)(heteroaryl); N(heteroaryl)-S(═O)₂N(aryl)(heteroaryl); SH; SCF₃; S—C₁₋₈-alkyl; S-benzyl; S-aryl; S-heteroaryl; S(═O)₂OH; S(═O)₂—OC₁₋₈-alkyl; S(═O)₂—O-aryl; S(═O)₂—O-heteroaryl; S(═O)C₁₋₈-alkyl; S(═O)aryl, S(═O)heteroaryl; S(═O)₂C₁₋₈-alkyl; S(═O)₂aryl; S(═O)₂heteroaryl; S(═O)₂NH₂; S(═O)₂NH—C₁₋₈-alkyl; S(═O)₂N(C₁₋₈-alkyl)₂; S(═O)₂NH-aryl; S(═O)₂N(aryl)₂; S(═O)₂NH-heteroaryl; S(═O)₂N(heteroaryl)₂; S(═O)₂N(C₁₋₈-alkyl)(aryl); S(═O)₂N(heteroaryl)(aryl); S(═O)₂N(C₁₋₈-alkyl)(heteroaryl); aryl, heteroaryl, C₃₋₇-cycloalkyl, heterocyclyl or C₁₋₈-alkyl-bridged aryl, heteroaryl, C₃₋₇-cycloalkyl or heterocyclyl. A substituent can itself optionally be mono- or poly-substituted. Polysubstitution is carried out with the same or with different substituents.

In connection with “aryl” and “heteroaryl”, “mono- or poly-substituted” is understood as meaning within the scope of this invention the substitution of one of more hydrogen atoms, in each case each independently, one or more times, for example two, three or four times, by substituents selected from the group comprising F; Cl; Br; I; NO; NO₂; CF₃; CN; R⁰; C(═O)H; C(═O)R⁰; CO₂H; C(═O)OR⁰; CONH₂; C(═O)NHR⁰; C(═O)N(R⁰)₂; OH; O—(C₁₋₈-alkyl)-O; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; S(═O)₂N(R⁰)₂ on one atom or optionally on different atoms, wherein a substituent can itself optionally be mono- or poly-substituted. Polysubstitution takes place with the same substituent or with different substituents.

Preferred “aryl” and “heteroaryl” substituents are F; Cl; Br; I; CN; NH₂; OCF₃; SCF₃; S(═O)CF₃; S(═O)₂CF₃; NH(OH); NO; NO₂; CF₂H; OCF₂H; SCF₂H; benzyl; C₁₋₈-alkyl; CF₃; C(═O)H; C(═O)OH; C(═O)C₁₋₈-alkyl; C(═O)aryl; C(═O)heteroaryl; C(═O)O—C₁₋₈-alkyl; C(═O)O-aryl; C(═O)O-heteroaryl; C(═O)NH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(aryl)(heteroaryl); OH; O—C₁₋₈-alkyl; O—C₁₋₈-alkyl-OH; O—(C₁₋₈-alkyl)-O; O-(C₁₋₈-alkyl)—O—C₁₋₈-alkyl; O-aryl; O-heteroaryl; O-benzyl; O—C(═O)—C₁₋₈-alkyl; O—C(═O)-aryl; O—C(═O)-heteroaryl; O—S(═O)₂OH; O—S(═O)₂—OC₁₋₈-alkyl; O—S(═O)₂—O-aryl; O—S(═O)₂—O-heteroaryl; O—S(═O)₂C₁₋₈-alkyl; O—S(═O)₂aryl; O—S(═O)₂heteroaryl; O—S(═O)₂NH₂; O—S(═O)₂NH—C₁₋₈-alkyl; O—S(═O)₂N(C₁₋₈-alkyl)₂; O—S(═O)₂NH-aryl; O—S(═O)₂N(aryl)₂; O—S(═O)₂NH-heteroaryl; O—S(═O)₂N(heteroaryl)₂; O—S(═O)₂N(C₁₋₈-alkyl)(aryl); O—S(═O)₂N(heteroaryl)(aryl); O—S(═O)₂N(C₁₋₈-alkyl)(heteroaryl); NH₂; NH(OH); N═C(NH₂)₂; NH—C₁₋₈-alkyl; NH—C₁₋₈-alkyl-OH; N(C₁₋₈-alkyl)₂; N(C₁₋₈-alkyl-OH)₂; NH-aryl; N(aryl)₂; NH-heteroaryl; N(heteroaryl)₂; N(C₁₋₈-alkyl)(aryl); N(C₁₋₈-alkyl)(heteroaryl); N(aryl)(heteroaryl); NH—C(═O)C₁₋₈-alkyl; NH—C(═O)-aryl; NH—C(═O)-heteroaryl; N(C₁₋₈-alkyl)-C(═O)C₁₋₈-alkyl; N(C₁₋₈-alkyl)-C(═O)aryl; N(C₁₋₈-alkyl)-C(═O)heteroaryl; N(aryl)-C(═O)C₁₋₈-alkyl; N(aryl)-C(═O)aryl; N(aryl)-C(═O)heteroaryl; N(heteroaryl)-C(═O)C₁₋₈-alkyl; N(heteroaryl)-C(═O)aryl; N(heteroaryl)-C(═O)heteroaryl; NH—S(═O)₂OH; NH—S(═O)₂C₁₋₈-alkyl; NH—S(═O)₂aryl; NH—S(═O)₂heteroaryl; NH—S(═O)₂OC₁₋₈-alkyl; NH—S(═O)₂O-aryl; NH—S(═O)₂O-heteroaryl; NH—S(═O)₂NH₂; NH—S(═O)₂NH(C₁₋₈-alkyl); NH—S(═O)₂NH(aryl); NH—S(═O)₂NH(heteroaryl); NH—S(═O)₂N(C₁₋₈-alkyl)₂; NH—S(═O)₂N(aryl)₂; NH—S(═O)₂N(heteroaryl)₂; NH—S(═O)₂N(C₁₋₈-alkyl)(aryl); NH—S(═O)₂N(C₁₋₈-alkyl)(heteroaryl); NH—S(═O)₂N(aryl)(heteroaryl); N(C₁₋₈-alkyl)-S(═O)₂OH; N(aryl)-S(═O)₂OH; N(heteroaryl)-S(═O)₂OH; N(C₁₋₈-alkyl)-S(═O)₂—C₁₋₈-alkyl; N(C₁₋₈-alkyl)-S(═O)₂-aryl; N(C₁₋₈-alkyl)-S(═O)₂-heteroaryl; N(aryl)-S(═O)₂—C₁₋₈-alkyl; N(aryl)-S(═O)₂-aryl; N(aryl)-S(═O)₂-heteroaryl; N(heteroaryl)-S(═O)₂—C₁₋₈-alkyl; N(heteroaryl)-S(═O)₂-aryl; N(heteroaryl)-S(═O)₂-heteroaryl; N(C₁₋₈-alkyl)-S(═O)₂—OC₁₋₈-alkyl; N(C₁₋₈-alkyl)-S(═O)₂—O-aryl; N(C₁₋₈-alkyl)-S(═O)₂—O-heteroaryl; N(aryl)-S(═O)₂—OC₁₋₈-alkyl; N(aryl)-S(═O)₂—O-aryl; N(aryl)-S(═O)₂—O-heteroaryl; N(heteroaryl)-S(═O)₂—OC₁₋₈-alkyl; N(heteroaryl)-S(═O)₂—O-aryl; N(heteroaryl)-S(═O)₂—O-heteroaryl; N(C₁₋₈-alkyl)-S(═O)₂NH₂; N(aryl)-S(═O)₂NH₂; N(heteroaryl)-S(═O)₂NH₂; N(C₁₋₈-alkyl)-S(═O)₂NH(C₁₋₈-alkyl); N(C₁₋₈-alkyl)-S(═O)₂NH(aryl); N(C₁₋₈-alkyl)-S(═O)₂NH(heteroaryl); N(C₁₋₈-alkyl)-S(═O)₂N(C₁₋₈-alkyl)₂; N(C₁₋₈-alkyl)-S(═O)₂N(aryl)₂; N(C₁₋₈-alkyl)-S(═O)₂N(heteroaryl)₂; N(C₁₋₈-alkyl)-S(═O)₂N(C₁₋₈-alkyl)(aryl); N(C₁₋₈-alkyl)-S(═O)₂N(C₁₋₈-alkyl)(heteroaryl); N(C₁₋₈-alkyl)-S(═O)₂N(aryl)(heteroaryl); N(aryl)-S(═O)₂NH(C₁₋₈-alkyl); N(aryl)-S(═O)₂NH(aryl); N(aryl)-S(═O)₂NH(heteroaryl); N(aryl)-S(═O)₂N(C₁₋₈-alkyl)₂; N(aryl)-S(═O)₂N(aryl)₂; N(aryl)-S(═O)₂N(heteroaryl)₂; N(aryl)-S(═O)₂N(C₁₋₈-alkyl)(aryl); N(aryl)-S(═O)₂N(C₁₋₈-alkyl)(heteroaryl); N(aryl)-S(═O)₂N(aryl)(heteroaryl); N(heteroaryl)-S(═O)₂NH(C₁₋₈-alkyl); N(heteroaryl)-S(═O)₂NH(aryl); N(heteroaryl)-S(═O)₂NH(heteroaryl); N(heteroaryl)-S(═O)₂N(C₁₋₈-alkyl)₂; N(heteroaryl)-S(═O)₂N(aryl)₂; N(heteroaryl)-S(═O)₂N(heteroaryl)₂; N(heteroaryl)-S(═O)₂N(C₁₋₈-alkyl)(aryl); N(heteroaryl)-S(═O)₂N(C₁₋₈-alkyl)(heteroaryl); N(heteroaryl)-S(═O)₂N(aryl)(heteroaryl); SH; S—C₁₋₈-alkyl; S-benzyl; S-aryl; S-heteroaryl; S(═O)₂OH; S(═O)₂—OC₁₋₈-alkyl; S(═O)₂—O-aryl; S(═O)₂—O-heteroaryl; S(═O)₂C₁₋₈-alkyl; S(═O)₂aryl, S(═O)₂heteroaryl; S(═O)C₁₋₈-alkyl; S(═O)aryl; S(═O)heteroaryl; S(═O)₂NH₂; S(═O)₂NH—C₁₋₈-alkyl; S(═O)₂N(C₁₋₈-alkyl)₂; S(═O)₂NH-aryl; S(═O)₂N(aryl)₂; S(═O)₂NH-heteroaryl; S(═O)₂N(heteroaryl)₂; S(═O)₂N(C₁₋₈-alkyl)(aryl); S(═O)₂N(heteroaryl)(aryl); S(═O)₂N(C₁₋₈-alkyl)(heteroaryl); aryl, heteroaryl, C₃₋₇-cycloalkyl, heterocyclyl or C₁₋₈-alkyl-bridged aryl, heteroaryl, C₃₋₇-cycloalkyl or heterocyclyl. A substituent can itself optionally be mono- or poly-substituted. Polysubstitution takes place with the same substituent or with different substituents. Particularly preferred substituents are F, Cl, OCH₃, CF₃, OCF₃, SCF₃ and CH₃.

The compounds according to the invention are defined by substituents, for example by R¹, R² and R³ (1st generation substituents), which are themselves optionally substituted (2nd generation substituents). Depending on the definition, these substituents of the substituents can in turn themselves be substituted (3rd generation substituents). If, for example, R¹═R⁰, wherein R⁰=aryl (1st generation substituent), aryl can itself be substituted, for example by NHR⁰, wherein R⁰═C₁₋₈-alkyl (2nd generation substituent). This yields the functional group aryl-NHC₁₋₈-alkyl. C₁₋₈-Alkyl can then in turn itself be substituted, for example by Cl (3rd generation substituent). Overall, this then yields the functional group aryl-NHC₁₋₈-alkyl-Cl.

In one preferred embodiment, however, the 3rd generation substituents cannot themselves be substituted, that is to say there are no 4th generation substituents.

In another preferred embodiment, the 2nd generation substituents cannot themselves be substituted, that is to say there are not even any 3rd generation substituents. In other words, the functional groups for R⁰ to R³⁵ in each case can optionally be substituted in this embodiment, but the substituents cannot themselves be substituted.

If a substituent occurs more than once within a molecule, such as, for example, the substituent R⁰, that substituent can have different meanings for different substituents: if, for example, both R¹═R⁰ and R⁷═R⁰, R⁰ can denote for R¹ and C₁₋₈-alkyl for R⁷.

In some cases, the compounds according to the invention are defined by substituents which, together with the carbon atom(s) or heteroatom(s) joining them as ring member or members, form a ring, for example a C₃₋₇-cycloalkyl or a heterocyclyl, in each case saturated or unsaturated, unsubstituted or mono- or poly-substituted. The ring systems so formed can optionally be fused with (hetero)aryl, that is to say with an aryl such as phenyl or with a heteroaryl such as pyridyl, it being possible for the (hetero)aryl radical to be unsubstituted or mono- or poly-substituted. The ring systems so formed are preferably fused with an aryl, particularly preferably with phenyl. If the substituents R¹¹ and R¹², for example, with the nitrogen atom joining them, form a piperidine ring, that piperidine ring can be fused with phenyl to give tetrahydroisoquinolinyl.

The expression “salt formed with a physiologically acceptable acid” is understood within the scope of this invention as meaning salts of the active ingredient in question with inorganic or organic acids that are physiologically acceptable—in particular when used in humans and/or mammals. The hydrochloride is particularly preferred. Examples of physiologically acceptable acids are: hydrochloric acid, hydrobromic acid, sulfuric acid, methanesulfonic acid, formic acid, acetic acid, oxalic acid, succinic acid, tartaric acid, mandelic acid, fumaric acid, maleic acid, lactic acid, citric acid, glutamic acid, saccharinic acid, monomethylsebacic acid, 5-oxo-proline, hexane-1-sulfonic acid, nicotinic acid, 2-, 3- or 4-aminobenzoic acid, 2,4,6-trimethyl-benzoic acid, α-liponic acid, acetylglycine, hippuric acid, phosphoric acid and/or aspartic acid. Citric acid and hydrochloric acid are particularly preferred.

Physiologically acceptable salts with cations or bases are salts of the compound in question—in the form of the anion with at least one, preferably inorganic cation—that are physiologically acceptable—in particular when used in humans and/or mammals. Particular preference is given to the salts of the alkali and alkaline earth metals but also to ammonium salts, but in particular to (mono-) or (di-)sodium, (mono-) or (di-)potassium, magnesium or calcium salts.

Preferred embodiments of the compounds according to the invention of formula (1) have the formula (1a), (1b) or (1c):

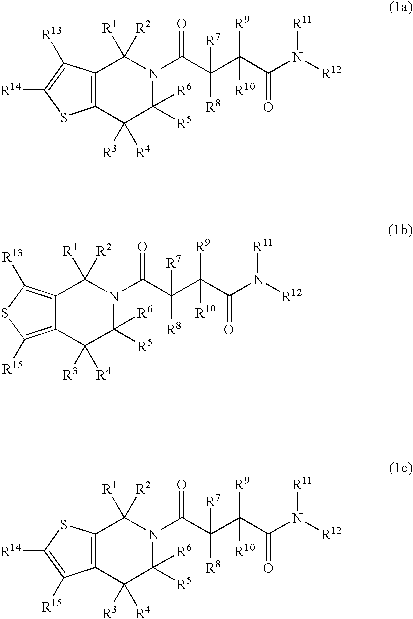

wherein R¹³, R¹⁴ and R¹⁵ each independently of the others denotes H; F; Cl; Br; I; NO; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; O—(C₁₋₈-alkyl)-O; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂.

Compounds of formulas (1a) and (1c) are particularly preferred. Compounds of formula (1a) are most particularly preferred.

Further preferred embodiments of the compounds according to the invention of formula (1) and of formulas (1a), (1b) and (1c) have the formula (2a), (2b) or (2c):

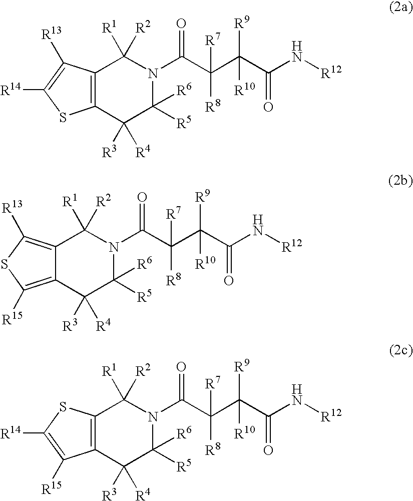

wherein R¹³, R¹⁴ and R¹⁵ each independently of the others denotes H; F; Cl; Br; I; NO; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; O—(C₁₋₈-alkyl)-O; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂.

Compounds of formulas (2a) and (2c) are particularly preferred. Compounds of formula (2a) are most particularly preferred.

In a further preferred embodiment of the compounds according to the invention, R¹³, R¹⁴ and R¹⁵ according to one of the formulas (1), (1a), (1b), (1c), (2a), (2b) or (2c) are in each case selected each independently from the group consisting of H; F; Cl; Br; I; NO; NO₂; CN; NH₂; NH—C₁₋₈-alkyl; N(C₁₋₈-alkyl)₂; NH—C(═O)C₁₋₈-alkyl; NH—C(═O)-aryl; NH—C(═O)-heteroaryl; C₁₋₈-alkyl; CF₃; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-O; O-(C₁₋₈-alkyl)-O—C₁₋₈alkyl; O-benzyl; O-aryl; O-heteroaryl; O—C(═O)C₁₋₈-alkyl; O—C(═O)aryl; O—C(═O)heteroaryl; SH; S—C₁₋₈-alkyl; SCF₃; S-benzyl; S-aryl; S-heteroaryl; aryl; heteroaryl; C₃₋₇-cycloalkyl; heterocyclyl or C₁₋₈-alkyl-bridged aryl, heteroaryl, C₃₋₇-cycloalkyl or heterocyclyl.

More preferably, the substituents R¹³, R¹⁴ and R¹⁵ according to one of the formulas (1), (1a), (1b), (1c), (2a), (2b) or (2c) are in each case selected each independently from the group consisting of H; F; Cl; Br; CN; NH₂; NH—C(═O)C₁₋₈-alkyl; NH—C(═O)-aryl; NH—C(═O)-heteroaryl; C₁₋₈-alkyl; CF₃; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O-benzyl; O-aryl; O-heteroaryl; SH; S—C₁₋₈-alkyl; SCF₃; S-benzyl; S-aryl; S-heteroaryl; aryl; heteroaryl; C₃₋₇-cycloalkyl; heterocyclyl or C₁₋₈-alkyl-bridged aryl, heteroaryl, C₃₋₇-cycloalkyl or heterocyclyl.

Yet more preferably, the substituents R¹³, R¹⁴ and R¹⁵ according to one of the formulas (1), (1a), (1b), (1c), (2a), (2b) or (2c) are in each case selected each independently from the group consisting of H; F; Cl; Br; CN; CF₃; OCF₃; SCF₃; C₁₋₈-alkyl; O—C₁₋₈-alkyl; CONH₂; C(═O)NH—O₁₋₈-alkyl; NH₂; NH—C(═O)C₁₋₈-alkyl.

Particularly preferably, the substituents R¹³, R¹⁴ and R¹⁵ are in each case selected each independently from the group consisting of H; F, Cl; Br; CN; OCH₃; OCF₃; CF₃ and C₁₋₈-alkyl.

Most particularly preferably, the substituents R¹³, R¹⁴ and R¹⁵ according to one of the formulas (1), (1a), (1b), (1c), (2a), (2b) or (2c) each independently of the others denotes H or CH₃.

Compounds in which one of R¹³ and R¹⁴; or R¹³ and R¹⁵; or R¹⁴ and R¹⁵ according to one of the formulas (1), (1a), (1b), (1c), (2a), (2b) or (2c) represents CH₃ and the other denotes H are particularly preferred.

In a further preferred embodiment, the substituent R¹ is selected from the group consisting of H; F; Cl; Br; CN; C₁₋₈-alkyl, saturated or unsaturated, branched or unbranched, unsubstituted or mono- or poly-substituted; C₃₋₇-cycloalkyl, saturated or unsaturated, unsubstituted or mono- or poly-substituted; aryl or heteroaryl, in each case unsubstituted or mono- or poly-substituted; C₁₋₈-alkyl-bridged C₃₋₇-cycloalkyl, saturated or unsaturated, unsubstituted or mono- or poly-substituted, wherein the alkyl chain in each case can be branched or unbranched, saturated or unsaturated, unsubstituted, mono- or poly-substituted; or C₁₋₈-alkyl-bridged aryl or heteroaryl, in each case unsubstituted or mono- or poly-substituted, wherein the alkyl chain in each case can be branched or unbranched, saturated or unsaturated, unsubstituted, mono- or poly-substituted; and the substituent R² is selected from the group consisting of H and C₁₋₈-alkyl, saturated or unsaturated, branched or unbranched, unsubstituted or mono- or poly-substituted; or

R¹ and R², together with the carbon atom joining them as ring member, form a C₃₋₇-cycloalkyl or heterocyclyl, in each case saturated or unsaturated, unsubstituted or mono- or poly-substituted, in each case optionally fused with (hetero)aryl, unsubstituted or mono- or poly-substituted.

In a further preferred embodiment, the substituent R¹ is selected from the group consisting of H; C₁₋₈-alkyl, saturated or unsaturated, branched or unbranched, unsubstituted or mono- or poly-substituted; C₃₋₇-cycloalkyl, saturated or unsaturated, unsubstituted or mono- or poly-substituted; aryl or heteroaryl, in each case unsubstituted or mono- or poly-substituted; C₁₋₈-alkyl-bridged C₃₋₇-cycloalkyl, saturated or unsaturated; or C₁₋₈-alkyl-bridged aryl or heteroaryl, unsubstituted or mono- or poly-substituted; and the substituent R² is selected from the group consisting of H and C₁₋₈-alkyl, saturated or unsaturated, branched or unbranched; or

R¹ and R², together with the carbon atom joining them as ring member, form a C₃₋₇-cycloalkyl, saturated or unsaturated, unsubstituted or mono- or poly-substituted, optionally fused with (hetero)aryl, unsubstituted or mono- or poly-substituted.

In a further preferred embodiment, the substituent R¹ is selected from the group consisting of H; C₁₋₈-alkyl, saturated or unsaturated, branched or unbranched, unsubstituted or mono- or poly-substituted; C₃₋₇-cycloalkyl, saturated or unsaturated, unsubstituted or mono- or poly-substituted; phenyl, pyridyl or thienyl, in each case unsubstituted or mono- or poly-substituted; C₁₋₈-alkyl-bridged C₃₋₇-cycloalkyl, saturated or unsaturated; or C₁₋₈-alkyl-bridged phenyl, pyridyl or thienyl, in each case unsubstituted or mono- or poly-substituted; and the substituent R² is selected from the group consisting of H and C₁₋₈-alkyl, saturated or unsaturated, branched or unbranched.

In a further preferred embodiment, the substituent R¹ is selected from the group consisting of H; C₁₋₈-alkyl, saturated or unsaturated, branched or unbranched, unsubstituted or mono- or poly-substituted; C₃₋₇-cycloalkyl, saturated or unsaturated, unsubstituted or mono- or poly-substituted; phenyl or thienyl, in each case unsubstituted or mono- or poly-substituted; C₁₋₈-alkyl-bridged C₃₋₇-cycloalkyl, saturated or unsaturated; or C₁₋₈-alkyl-bridged phenyl or thienyl; in each case unsubstituted or mono- or poly-substituted; and the substituent R² represents H.

In a further preferred embodiment, the substituent R¹ is selected from the group consisting of H; C₁₋₈-alkyl, saturated, branched or unbranched, unsubstituted or mono- or poly-substituted; C₃₋₇-cycloalkyl, saturated, unsubstituted or mono- or poly-substituted; phenyl or thienyl, in each case unsubstituted or mono- or poly-substituted; or C₁₋₈-alkyl-bridged phenyl, unsubstituted; and the substituent R² represents H.

In a further preferred embodiment, the substituent R¹ is selected from the group consisting of C₁₋₈-alkyl, saturated, branched or unbranched, unsubstituted; and C₁₋₈-alkyl-bridged phenyl, unsubstituted; or

denotes (CH₂)ₑ-bridged phenyl according to one of the following general formulas (3a) or (4a):

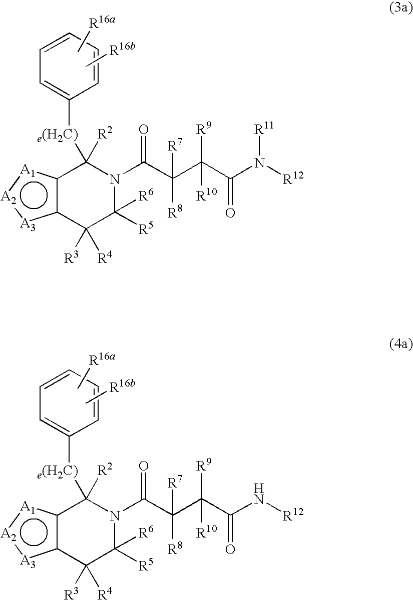

mono- or di-substituted by R^{16a} and/or R^{16b}; or

denotes according to one of the following formulas (3b) or (4b) (CH₂)ₑ-bridged thienyl

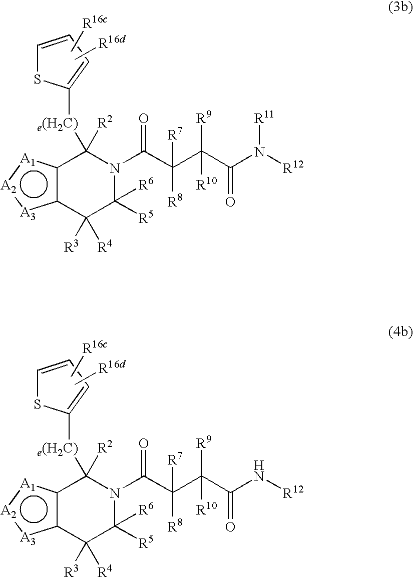

mono- or di-substituted by R^{16c} and/or R^{16d}; or

denotes (CH₂)ₑ-bridged C₃₋₇-cycloalkyl according to one of the following formulas (3c) or (4c):

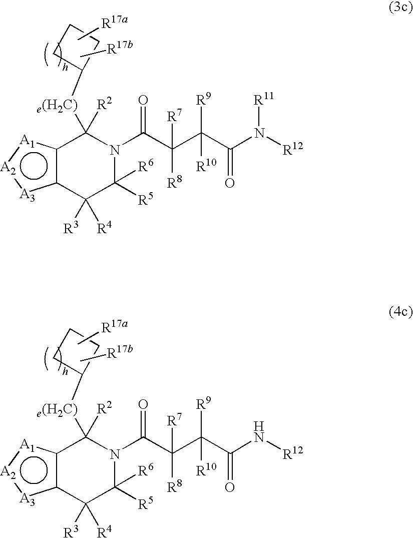

mono- or di-substituted by R^{17a} and/or R^{17b};
and the substituent R² represents H;
wherein e is 0, 1, 2, 3 or 4, preferably 0;

R^{16a} and R^{16b} are selected each independently from the group consisting of H, F, Cl, Br, CN, NH₂, OCF₃, SCF₃, CF₃, C₁₋₈-alkyl, saturated or unsaturated, branched or unbranched, unsubstituted or mono- or poly-substituted; aryl, heteroaryl, in each case unsubstituted or mono- or poly-substituted; C₃₋₇-cycloalkyl or heterocyclyl, in each case saturated or unsaturated, unsubstituted or mono- or poly-substituted;

R^{16c} and R^{16d} are selected each independently from the group consisting of H, F, Cl, Br, CN, NH₂, OCF₃, SCF₃, CF₃, C₁₋₈-alkyl, saturated or unsaturated, branched or unbranched, unsubstituted or mono- or poly-substituted; aryl, heteroaryl, in each case unsubstituted or mono- or poly-substituted; C₃₋₇-cycloalkyl or heterocyclyl, in each case saturated or unsaturated, unsubstituted or mono- or poly-substituted;

h is 0, 1, 2, 3 or 4;

R^{17a} and R^{17b} are selected each independently from the group consisting of H, F, Cl, Br, CN, NH₂, OCF₃, SCF₃, CF₃, C₁₋₈-alkyl, saturated or unsaturated, branched or unbranched, unsubstituted or mono- or poly-substituted; aryl, heteroaryl, in each case unsubstituted or mono- or poly-substituted.

R^{16a} and R^{16b} are preferably selected each independently from the group consisting of H, F, Cl, Br, CH₃, C₂N₅, isopropyl, OCH₃ and CF₃.

R^{16c} and R^{16d} are preferably selected each independently from the group consisting of H, F, Cl, Br, CH₃, OCH₃ and CF₃. Particularly preferably R^{16c} and R^{16d} each independently represent H or CH₃.

h preferably represents 2 or 3, particularly preferably 3.

R^{17a} and R^{17b} are preferably selected each independently from the group consisting of H, F, Cl, Br, CH₃, OCH₃ and CF₃. Particularly preferably R^{17a} and R^{17b} each represent H.

Compounds of formulas (3a), (4a), (3b) and (4b) are most particularly preferred. Compounds of formulas (4a) and (4b) are especially preferred.

In another preferred embodiment, R¹ and R² each represents H, or R² denotes H and R¹ is not H.

In a further preferred embodiment, the substituents R³, R⁴, R⁵ and R⁶ are selected each independently from the group consisting of H, C₁₋₈-alkyl, saturated or unsaturated, branched or unbranched, unsubstituted or mono- or poly-substituted; phenyl, unsubstituted or mono- or poly-substituted. Preferably, R³, R⁴, R⁵ and R⁶ each independently of the others represents H; CH₃; or phenyl. Particularly preferably, R³, R⁴, R⁵ each denotes H and R⁶ is selected from H and phenyl.

In a further preferred embodiment, the substituent R⁷ is selected from the group consisting of H; F; Cl; Br; CN; OH; NH₂; C₁₋₈-alkyl, O—C₁₋₈-alkyl, NH—C₁₋₈-alkyl, N(C₁₋₈-alkyl)₂, in each case saturated or unsaturated, branched or unbranched, unsubstituted or mono- or poly-substituted; phenyl or heteroaryl, in each case unsubstituted or mono- or poly-substituted; C₁₋₂-alkyl-bridged phenyl or heteroaryl, in each case unsubstituted or mono- or poly-substituted, wherein the alkyl chain in each case can be branched or unbranched, saturated or unsaturated, unsubstituted or mono- or poly-substituted, and R⁹ is selected from the group consisting of H; F; Cl; Br; CN; OH; NH₂; C₁₋₈-alkyl, O—C₁₋₈-alkyl, NH—C₁₋₈-alkyl, N(C₁₋₈-alkyl)₂, in each case saturated or unsaturated, branched or unbranched, unsubstituted or mono- or poly-substituted; phenyl or heteroaryl, in each case unsubstituted or mono- or poly-substituted; C₂-alkyl-bridged phenyl or heteroaryl, in each case unsubstituted or mono- or poly-substituted, wherein the alkyl chain in each case can be branched or unbranched, saturated or unsaturated, unsubstituted or mono- or poly-substituted.

Preferably, R⁷ is selected from the group consisting of H; C₁₋₈-alkyl, NH—C₁₋₈-alkyl, N(C₁₋₈-alkyl)₂, in each case saturated or unsaturated, branched or unbranched, unsubstituted or mono- or poly-substituted; phenyl or heteroaryl, in each case unsubstituted or mono- or poly-substituted; C₁₋₂-alkyl-bridged phenyl or heteroaryl, in each case unsubstituted or mono- or poly-substituted, wherein the alkyl chain in each case can be branched or unbranched, saturated or unsaturated, unsubstituted or mono- or poly-substituted, and R⁹ is selected from the group consisting of H; C₁₋₈-alkyl, NH—C₁₋₈-alkyl, N(C₁₋₈-alkyl)₂, in each case saturated or unsaturated, branched or unbranched, unsubstituted or mono- or poly-substituted; phenyl or heteroaryl, in each case unsubstituted or mono- or poly-substituted; C₂-alkyl-bridged phenyl or heteroaryl, in each case unsubstituted or mono- or poly-substituted, wherein the alkyl chain in each case can be branched or unbranched, saturated or unsaturated, unsubstituted or mono- or poly-substituted.

More preferably, R⁷ is selected from the group consisting of H; C₁₋₈-alkyl, in each case saturated or unsaturated, branched or unbranched, unsubstituted or mono- or poly-substituted; phenyl, unsubstituted or mono- or poly-substituted; C₁₋₂-alkyl-bridged phenyl, unsubstituted or mono- or poly-substituted, wherein the alkyl chain in each case can be branched or unbranched, saturated or unsaturated, and R⁹ is selected from the group consisting of H; C₁₋₈-alkyl, in each case saturated or unsaturated, branched or unbranched, unsubstituted or mono- or poly-substituted; phenyl, unsubstituted or mono- or poly-substituted; C₂-alkyl-bridged phenyl, unsubstituted or mono- or poly-substituted, wherein the alkyl chain in each case can be branched or unbranched, saturated or unsaturated.

Yet more preferably, R⁷ is selected from the group consisting of H; C₁₋₈-alkyl, in each case saturated or unsaturated, branched or unbranched; phenyl or benzyl, in each case unsubstituted or mono- or poly-substituted, and R⁹ is selected from the group consisting of H; C₁₋₈-alkyl, in each case saturated or unsaturated, branched or unbranched; phenyl, unsubstituted or mono- or poly-substituted. Particularly preferably, one of R⁷ and R⁹ is selected from the group consisting of H; CH₃; phenyl, unsubstituted or mono- or poly-substituted; and the other represents H. Especially preferably, one of R⁷ and R⁹ is selected from the group consisting of H; CH₃; phenyl, unsubstituted; and the other represents H.

In another preferred embodiment, R⁷ and R⁹, together with carbon atoms joining them as ring members, form a C₃₋₇-cycloalkyl or piperidinyl, in each case saturated or unsaturated, unsubstituted or mono- or poly-substituted, optionally fused with (hetero)aryl, unsubstituted or mono- or poly-substituted. R⁷ and R⁹, together with the carbon atoms joining them as ring members, preferably form a C₃₋₇-cycloalkyl, saturated or unsaturated, unsubstituted or mono- or poly-substituted, optionally fused with aryl, unsubstituted or mono- or poly-substituted. R⁷ and R⁹, together with the carbon atoms joining them as ring members, particularly preferably form a C₃₋₇-cycloalkyl, saturated or unsaturated, optionally fused with phenyl.

In a further preferred embodiment, R⁸ is selected from the group consisting of H; and C₁₋₈-alkyl, saturated or unsaturated, branched or unbranched, unsubstituted or mono- or poly-substituted. R⁸ is preferably selected from H and C₁₋₈-alkyl, saturated. More preferably, R⁸ represents H or CH₃. R⁸ particularly preferably denotes H.

In a further preferred embodiment, R¹⁰ is selected from the group consisting of H; and C₁₋₈-alkyl, saturated or unsaturated, branched or unbranched, unsubstituted or mono- or poly-substituted. R¹⁰ is preferably selected from H and C₁₋₈-alkyl, saturated. More preferably, R¹⁰ represents H or CH₃. R¹⁰ particularly preferably denotes H.

In a further preferred embodiment, R⁷ and R⁸; or R⁹ and R¹⁰; together with the carbon atoms joining them as ring members, form a C₃₋₇-cycloalkyl or piperidinyl, in each case saturated or unsaturated, unsubstituted or mono- or poly-substituted, optionally fused with (hetero)aryl, unsubstituted or mono- or poly-substituted. R⁷ and R⁸; or R⁹ and R¹⁰; together with the carbon atoms joining them as ring members, preferably form a C₃₋₇-cycloalkyl, saturated or unsaturated, unsubstituted or mono- or poly-substituted, optionally fused with aryl, unsubstituted or mono- or poly-substituted. Particularly preferably, R⁷ and R⁸; or R⁹ and R¹⁰; together with the carbon atoms joining them as ring members, form a C₃₋₇-cycloalkyl, saturated or unsaturated, optionally fused with phenyl.

In a further preferred embodiment, the substituent R¹¹ is selected from the group consisting of H; C₁₋₈-alkyl, saturated or unsaturated, branched or unbranched, C₃₋₇-cycloalkyl, saturated or unsaturated; and benzyl, unsubstituted or mono- or poly-substituted. R¹¹ preferably represents H; C₁₋₈-alkyl, saturated or unsaturated, branched or unbranched; or benzyl. More preferably, R¹¹ denotes H or CH₃. R¹¹ particularly preferably represents H.

In a further preferred embodiment, R¹² is selected from the group consisting of C₄₋₁₆-alkyl, saturated or unsaturated; branched or unbranched, unsubstituted or mono- or poly-substituted; C₃₋₇-cycloalkyl or heterocyclyl, in each case saturated or unsaturated, unsubstituted or mono- or poly-substituted; aryl or heteroaryl, unsubstituted or mono- or poly-substituted; C₁₋₈-alkyl-bridged C₃₋₇-cycloalkyl or heterocyclyl, in each case saturated or unsaturated, unsubstituted or mono- or poly-substituted, wherein the alkyl chain in each case can be branched or unbranched, saturated or unsaturated, unsubstituted, mono- or poly-substituted; C₁₋₈-alkyl-bridged aryl or heteroaryl, in each case unsubstituted or mono- or poly-substituted, wherein the alkyl chain in each case can be branched or unbranched, saturated or unsaturated, unsubstituted, mono- or poly-substituted. More preferably, R¹² is selected from the group consisting of C₄₋₁₆-alkyl, saturated or unsaturated, branched or unbranched, unsubstituted or mono- or poly-substituted; C₃₋₇-cycloalkyl or heterocyclyl, in each case saturated or unsaturated, unsubstituted or mono- or poly-substituted; aryl or heteroaryl, unsubstituted or mono- or poly-substituted; C₁₋₈-alkyl-bridged C₃₋₇-cycloalkyl or heterocyclyl, in each case saturated or unsaturated, unsubstituted or mono- or poly-substituted, wherein the alkyl chain in each case can be branched or unbranched, saturated or unsaturated, unsubstituted, mono- or poly-substituted; C₁₋₈-alkyl-bridged aryl or heteroaryl, in each case unsubstituted or mono- or poly-substituted, wherein the alkyl chain in each case can be branched or unbranched, saturated or unsaturated, unsubstituted, mono- or poly-substituted; with the proviso that when R¹² denotes heterocyclyl or heteroaryl, bonding of the heterocyclyl or heteroaryl takes place via a carbon atom of the heterocyclyl or heteroaryl.

Yet more preferably, R¹² represents C₄₋₁₆-alkyl, saturated or unsaturated, branched or unbranched, unsubstituted or mono- or poly-substituted; or is selected from the following partial structures A, B and C

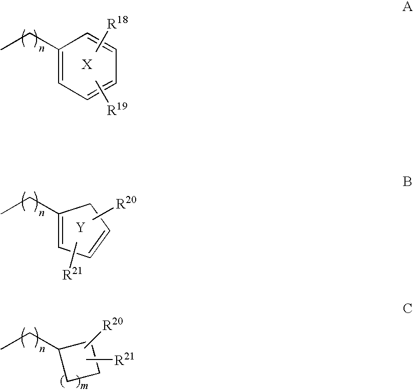

wherein
n=0, 1, 2, 3, 4, 5, 6, 7 or 8; particularly preferably 1, 2 or 3, especially 1;
m=0, 1, 2 or 3;
the ring X can contain one or two N atoms as ring member(s);
the ring Y contains at least 1 heteroatom selected from N, O and S and can contain up to 3 heteroatoms selected each independently from N, O and S;
R¹⁸ and R¹⁹ each independently denote H; F; Cl; Br; I; NO; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; O—(C₁₋₈-alkyl)-O; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂; or
R¹⁸ and R¹⁹, together with the carbon or nitrogen atoms joining them as ring members, form an aryl or heteroaryl fused with the phenyl ring and in each case unsubstituted or mono- or poly-substituted; or a C₃₋₇-cycloalkyl or heterocyclyl fused with the phenyl ring and in each case saturated or unsaturated, unsubstituted or mono- or poly-substituted;
R²⁰ and R²¹ each independently denote H or C₁₋₈-alkyl, saturated or unsaturated, branched or unbranched, unsubstituted or mono- or poly-substituted; C₃₋₇-cycloalkyl or heterocyclyl, in each case saturated or unsaturated, unsubstituted or mono- or poly-substituted.

Particular preference is given to compounds in which R¹² represents the partial structure A. Most particular preference is given to compounds in which R¹² represents the partial structure A wherein

R¹⁸ and R¹⁹ each independently denote H; F; Cl; Br; I; CN; NH₂; OCF₃; SCF₃; S(═O)CF₃; S(═O)₂CF₃; NH(OH); NO; NO₂; CF₂H; OCF₂H; SCF₂H; benzyl; C₁₋₈-alkyl; CF₃; C(═O)H; C(═O)OH; C(═O)C₁₋₈-alkyl; C(═O)aryl; C(═O)heteroaryl; C(═O)O—C₁₋₈-alkyl; C(═O)O-aryl; C(═O)O-heteroaryl; C(═O)NH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(aryl)(heteroaryl); OH; O—C₁₋₈-alkyl; O—C₁₋₈-alkyl-OH; O—(C₁₋₈-alkyl)-O; O—(C₁₋₈-alkyl)-O—C₁₋₈-alkyl; O-aryl; O-heteroaryl; O-benzyl; O—C(═O)—C₁₋₈-alkyl; O—C(═O)-aryl; O—C(═O)-heteroaryl; O—S(═O)₂OH; O—S(═O)₂—OC₁₋₈-alkyl; O—S(═O)₂—O-aryl; O—S(═O)₂—O-heteroaryl; O—S(═O)₂C₁₋₈-alkyl; O—S(═O)₂aryl; O—S(═O)₂heteroaryl; O—S(═O)₂NH₂; O—S(═O)₂NH—C₁₋₈-alkyl; O—S(═O)₂N(C₁₋₈-alkyl)₂; O—S(═O)₂NH-aryl; O—S(═O)₂N(aryl)₂; O—S(═O)₂NH-heteroaryl; O—S(═O)₂N(heteroaryl)₂; O—S(═O)₂N(C₁₋₈-alkyl)(aryl); O—S(═O)₂N(heteroaryl)(aryl); O—S(═O)₂N(C₁₋₈-alkyl)(heteroaryl); NH₂; NH(OH); N═C(NH₂)₂; NH—C₁₋₈-alkyl; NH—C₁₋₈-alkyl-OH; N(C₁₋₈-alkyl)₂; N(C₁₋₈-alkyl-OH)₂; NH-aryl; N(aryl)₂; NH-heteroaryl; N(heteroaryl)₂; N(C₁₋₈-alkyl)(aryl); N(C₁₋₈-alkyl)(heteroaryl); N(aryl)(heteroaryl); NH—C(═O)C₁₋₈-alkyl; NH—C(═O)-aryl; NH—C(═O)-heteroaryl; N(C₁₋₈-alkyl)-C(═O)C₁₋₈-alkyl; N(C₁₋₈-alkyl)-C(═O)aryl; N(C₁₋₈-alkyl)-C(═O)heteroaryl; N(aryl)-C(═O)C₁₋₈-alkyl; N(aryl)-C(═O)aryl; N(aryl)-C(═O)heteroaryl; N(heteroaryl)-C(═O)C₁₋₈-alkyl; N(heteroaryl)-C(═O)aryl; N(heteroaryl)-C(═O)heteroaryl; NH—S(═O)₂OH; NH—S(═O)₂C₁₋₈-alkyl; NH—S(═O)₂aryl; NH—S(═O)₂heteroaryl; NH—S(═O)₂OC₁₋₈-alkyl; NH—S(═O)₂O-aryl; NH—S(═O)₂O-heteroaryl; NH—S(═O)₂NH₂; NH—S(═O)₂NH(C₁₋₈-alkyl); NH—S(═O)₂NH(aryl); NH—S(═O)₂NH(heteroaryl); NH—S(═O)₂N(C₁₋₈-alkyl)₂; NH—S(═O)₂N(aryl)₂; NH—S(═O)₂N(heteroaryl)₂; NH—S(═O)₂N(C₁₋₈-alkyl)(aryl); NH—S(═O)₂N(C₁₋₈-alkyl)(heteroaryl); NH—S(═O)₂N(aryl)(heteroaryl); N(C₁₋₈-alkyl)-S(═O)₂OH; N(aryl)-S(═O)₂OH; N(heteroaryl)-S(═O)₂OH; N(C₁₋₈-alkyl)-S(═O)₂—C₁₋₈-alkyl; N(C₁₋₈-alkyl)-S(═O)₂-aryl; N(C₁₋₈-alkyl)-S(═O)₂-heteroaryl; N(aryl)-S(═O)₂—C₁₋₈-alkyl; N(aryl)-S(═O)₂-aryl; N(aryl)-S(═O)₂-heteroaryl; N(heteroaryl)-S(═O)₂—C₁₋₈-alkyl; N(heteroaryl)-S(═O)₂-aryl; N(heteroaryl)-S(═O)₂-heteroaryl; N(C₁₋₈-alkyl)-S(═O)₂—OC₁₋₈-alkyl; N(C₁₋₈-alkyl)-S(═O)₂—O-aryl; N(C₁₋₈-alkyl)-S(═O)₂—O-heteroaryl; N(aryl)-S(═O)₂—OC₁₋₈-alkyl; N(aryl)-S(═O)₂—O-aryl; N(aryl)-S(═O)₂—O-heteroaryl; N(heteroaryl)-S(═O)₂—OC₁₋₈-alkyl; N(heteroaryl)-S(═O)₂—O-aryl; N(heteroaryl)-S(═O)₂—O-heteroaryl; N(C₁₋₈-alkyl)-S(═O)₂NH₂; N(aryl)-S(═O)₂NH₂; N(heteroaryl)-S(═O)₂NH₂; N(C₁₋₈-alkyl)-S(═O)₂NH(C₁₋₈-alkyl); N(C₁₋₈-alkyl)-S(═O)₂NH(aryl); N(C₁₋₈-alkyl)-S(═O)₂NH(heteroaryl); N(C₁₋₈-alkyl)-S(═O)₂N(C₁₋₈-alkyl)₂; N(C₁₋₈-alkyl)-S(═O)₂N(aryl)₂; N(C₁₋₈-alkyl)-S(═O)₂N(heteroaryl)₂; N(C₁₋₈-alkyl)-S(═O)₂N(C₁₋₈-alkyl)(aryl); N(C₁₋₈-alkyl)-S(═O)₂N(C₁₋₈-alkyl)(heteroaryl); N(C₁₋₈-alkyl)-S(═O)₂N(aryl)(heteroaryl); N(aryl)-S(═O)₂NH(C₁₋₈-alkyl); N(aryl)-S(═O)₂NH(aryl); N(aryl)-S(═O)₂NH(heteroaryl); N(aryl)-S(═O)₂N(C₁₋₈-alkyl)₂; N(aryl)-S(═O)₂N(aryl)₂; N(aryl)-S(═O)₂N(heteroaryl)₂; N(aryl)-S(═O)₂N(C₁₋₈-alkyl)(aryl); N(aryl)-S(═O)₂N(C₁₋₈-alkyl)(heteroaryl); N(aryl)-S(═O)₂N(aryl)(heteroaryl); N(heteroaryl)-S(═O)₂NH(C₁₋₈-alkyl); N(heteroaryl)-S(═O)₂NH(aryl); N(heteroaryl)-S(═O)₂NH(heteroaryl); N(heteroaryl)-S(═O)₂N(C₁₋₈-alkyl)₂; N(heteroaryl)-S(═O)₂N(aryl)₂; N(heteroaryl)-S(═O)₂N(heteroaryl)₂; N(heteroaryl)-S(═O)₂N(C₁₋₈-alkyl)(aryl); N(heteroaryl)-S(═O)₂N(C₁₋₈-alkyl)(heteroaryl); N(heteroaryl)-S(═O)₂N(aryl)(heteroaryl); SH; S—C₁₋₈-alkyl; S-benzyl; S-aryl; S-heteroaryl; S(═O)₂OH; S(═O)₂—OC₁₋₈-alkyl; S(═O)₂—O-aryl; S(═O)₂—O-heteroaryl; S(═O)₂C₁₋₈-alkyl; S(═O)₂aryl, S(═O)₂heteroaryl; S(═O)C₁₋₈-alkyl; S(═O)aryl; S(═O)heteroaryl; S(═O)₂NH₂; S(═O)₂NH—C₁₋₈-alkyl; S(═O)₂N(C₁₋₈-alkyl)₂; S(═O)₂NH-aryl; S(═O)₂N(aryl)₂; S(═O)₂NH-heteroaryl; S(═O)₂N(heteroaryl)₂; S(═O)₂N(C₁₋₈-alkyl)(aryl); S(═O)₂N(heteroaryl)(aryl); S(═O)₂N(C₁₋₈-alkyl)(heteroaryl); aryl, heteroaryl, C₃₋₇-cycloalkyl, heterocyclyl or C₁₋₈-alkyl-bridged aryl, heteroaryl, C₃₋₇-cycloalkyl or heterocyclyl; or

R¹⁸ and R¹⁹, together with the carbon or nitrogen atoms joining them as ring members, form an aryl or heteroaryl group fused with the phenyl ring and in each case unsubstituted or mono- or poly-substituted; or a C₃₋₇-cycloalkyl or heterocyclyl group fused with the phenyl ring and in each case saturated or unsaturated, unsubstituted or mono- or poly-substituted.

In a further preferred embodiment, R¹² represents C₄₋₁₈-alkyl, saturated or unsaturated, branched or unbranched, or is selected from the following partial structures A, B and C

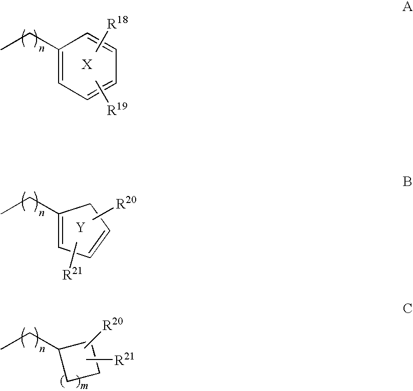

wherein
n=0, 1, 2, 3, 4, 5, 6, 7 or 8; particularly preferably 1, 2 or 3, especially 1;
m=0, 1, 2 or 3;
the ring X can contain a N atom as ring member;
the ring Y contains at least 1 heteroatom selected from N, O and S and can contain up to 3 heteroatoms selected each independently from N, O and S;
R¹⁸ and R¹⁹ each independently denote H; F; Cl; Br; I; NO; NO₂; CN; NH₂; NH—C₁₋₈-alkyl; N(C₁₋₈-alkyl)₂; NH—C(═O)C₁₋₈-alkyl; NH—C(═O)-aryl; NH—C(═O)-heteroaryl; C₁₋₈-alkyl; CF₃; C(═O)H; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(aryl)(heteroaryl); OH; O—C₁₋₈-alkyl; O—C₁₋₈-alkyl-OH; OCF₃; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; SH; S—C₁₋₈-alkyl; SCF₃; S-benzyl; S-aryl; S-heteroaryl; aryl; heteroaryl; C₃₋₇-cycloalkyl; heterocyclyl; or C₁₋₈-alkyl-bridged aryl, heteroaryl, C₃₋₇-cycloalkyl or heterocyclyl; or
R¹⁸ and R¹⁹, together with the carbon or nitrogen atoms joining them as ring members, form a C₃₋₇-cycloalkyl fused with the phenyl ring and saturated or unsaturated, unsubstituted or mono- or poly-substituted; or a phenyl, imidazolyl or thiadiazolyl fused with the phenyl ring and in each case unsubstituted or mono- or poly-substituted; or together with the carbon atoms joining them as ring members form O—CH₂—O; or O—CH₂—CH₂—O;
R²⁰ and R²¹ each independently denote H; or C₁₋₈-alkyl, saturated or unsaturated, branched or unbranched.

Particular preference is given to compounds in which R¹² represents the partial structure A. Most particular preference is given to compounds in which R¹² represents the partial structure A wherein R¹⁸ and R¹⁹ each independently denote H; F; Cl; Br; CN; NH₂; C₁₋₈-alkyl; CF₃; OH; O—C₁₋₈-alkyl; OCF₃; or SCF₃, or R¹⁸ and R¹⁹, together with the carbon or nitrogen atoms joining them as ring members, form a C₃₋₇-cycloalkyl fused with the phenyl ring and saturated or unsaturated, unsubstituted or mono- or poly-substituted; or a phenyl, imidazolyl or thiadiazolyl fused with the phenyl ring and in each case unsubstituted or mono- or poly-substituted; or together with the carbon atoms joining them as ring members form O—CH₂—O; or O—CH₂—CH₂—O.

In a further preferred embodiment, R¹² represents C₄₋₁₆-alkyl, saturated or unsaturated, branched or unbranched, or is selected from the following partial structures A, B and C

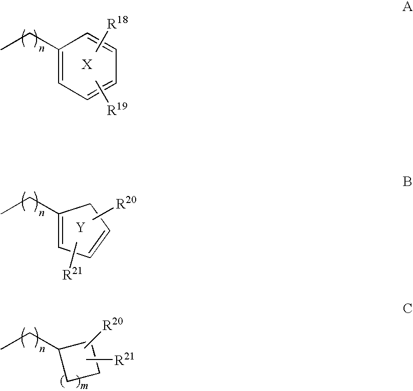

wherein
n=0, 1, 2, 3, 4, 5 or 6; particularly preferably 1, 2 or 3, especially 1;
m=0, 1, 2 or 3;
the ring X can contain a N atom as ring member;
the ring Y contains at least 1 heteroatom selected from N, O and S and can contain up to 3 heteroatoms selected each independently from N, O and S;
R¹⁸ and R¹⁹ each independently denote H; F; Cl; CN; CH₃; CF₃; OH; OCH₃; OCF₃; or SCF₃; or
R¹⁸ and R¹⁹, together with the carbon or nitrogen atoms joining them as ring members, form a phenyl, imidazole, thiadiazole fused with the phenyl ring and in each case unsubstituted or mono- or poly-substituted; or O—CH₂—O; or O—CH₂—CH₂—O; and
R²⁰ and R²¹ each independently denote H; or CH₃.

Particular preference is given to compounds in which R¹² represents the partial structure A. Most particular preference is given to compounds in which R¹² represents the partial structure A wherein R¹⁸ and R¹⁹ each independently denote H; F; Cl; CN; CH₃; CF₃; OH; OCH₃; OCF₃; or SCF₃.

In another preferred embodiment, R¹¹ and R¹², together with the nitrogen atom joining them as ring member, form one of the following groups

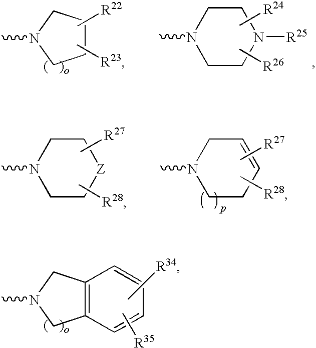

wherein
Z represents O or S;
o is 0, 1 or 2;
p is 0 or 1;
R²², R²³, R²⁴, R²⁵ and R²⁶ each independently denote H; C₁₋₈-alkyl, saturated or unsaturated; branched or unbranched, unsubstituted or mono- or poly-substituted; C₃₋₇-cycloalkyl or heterocyclyl, in each case saturated or unsaturated, unsubstituted or mono- or poly-substituted; aryl or heteroaryl, in each case unsubstituted or mono- or poly-substituted; C₁₋₈-alkyl-bridged C₃₋₇-cycloalkyl or heterocyclyl, in each case saturated or unsaturated, unsubstituted or mono- or poly-substituted, wherein the alkyl chain in each case can be branched or unbranched, saturated or unsaturated, unsubstituted, mono- or poly-substituted; C₁₋₈-alkyl-bridged aryl or heteroaryl, in each case unsubstituted or mono- or poly-substituted, wherein the alkyl chain in each case can be branched or unbranched, saturated or unsaturated, mono- or poly-substituted; or
R²² and R²³, with the carbon atom(s) joining them as ring member(s), form a C₃₋₇-cycloalkyl or heterocyclyl, in each case saturated or unsaturated, unsubstituted or mono- or poly-substituted, optionally fused with (hetero)aryl, unsubstituted or mono- or poly-substituted; or form a fused aryl or heteroaryl, unsubstituted or mono- or poly-substituted;
R²⁷ and R²⁸ each independently denote H; C₁₋₈-alkyl, saturated or unsaturated, branched or unbranched, unsubstituted or mono- or poly-substituted; and
R³⁴ and R³⁵ each independently denote H; F; Cl; Br; I; NO; NO₂; CN; NH₂; NH—C₁₋₈-alkyl; N(C₁₋₈-alkyl)₂; NH—C(═O)C₁₋₈-alkyl; NH—C(═O)-aryl; NH—C(═O)-heteroaryl; C₁₋₈-alkyl; CF₃; C(═O)H; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(aryl)(heteroaryl); OH; O—C₁₋₈-alkyl; O—C₁₋₈-alkyl-OH; OCF₃; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; SH; S—C₁₋₈-alkyl; SCF₃; S-benzyl; S-aryl; S-heteroaryl; aryl; heteroaryl; C₃₋₇-cycloalkyl; heterocyclyl; or C₁₋₈-alkyl-bridged aryl, heteroaryl, C₃₋₇-cycloalkyl or heterocyclyl.

R¹¹ and R¹², together with the nitrogen atom joining them as ring member, preferably form one of the following groups:

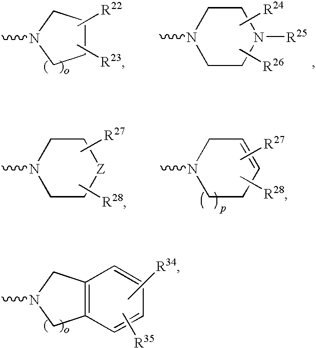

wherein
Z represents O or S;
o is 0, 1 or 2;
p is 0 or 1;
R²², R²³ and R²⁵ are each independently selected from the group consisting of H; C₁₋₈-alkyl, saturated or unsaturated; branched or unbranched; or form one of the following groups:

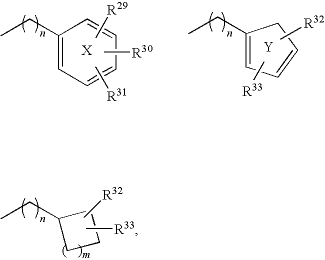

wherein
n is 0, 1, 2, 3 or 4;
m is 0, 1, 2 or 3;
the ring X can contain a N atom;
the ring Y can contain up to 3 heteroatoms selected each independently from N, O and S;
R²⁹, R³⁰ and R³¹ each independently denote H; F; Cl; Br; CN; NH₂; C₁₋₈-alkyl; CF₃; OH; O—C₁₋₈-alkyl; OCF₃; or SCF₃; or
R²⁹ and R³⁰, together with the carbon atoms joining them as ring members, form a C₃₋₇-cycloalkyl group fused with the phenyl ring, saturated or unsaturated; or a phenyl fused with the phenyl ring, unsubstituted or mono- or poly-substituted; or O—CH₂—O; or O—CH₂—CH₂—O;
R³² and R³³ each independently denote H; or C₁₋₈-alkyl, saturated or unsaturated, branched or unbranched; or
R²² and R²³ together with the carbon atom(s) joining them as ring member(s), form a C₃₋₇-cycloalkyl or heterocyclyl group, in each case saturated or unsaturated, optionally fused with (hetero)aryl, unsubstituted or mono- or poly-substituted; or form a fused aryl or heteroaryl, unsubstituted or mono- or poly-substituted;
R²⁴ and R²⁶ each independently denote H; C₁₋₈-alkyl, saturated or unsaturated; branched or unbranched, unsubstituted or mono- or poly-substituted; aryl or heteroaryl, in each case unsubstituted or mono- or poly-substituted; C₁₋₈-alkyl-bridged aryl or heteroaryl, in each case unsubstituted or mono- or poly-substituted, wherein the alkyl chain in each case can be branched or unbranched, saturated or unsaturated, unsubstituted, mono- or poly-substituted;
R²⁷ and R²⁸ each independently denote H; C₁₋₈-alkyl, saturated or unsaturated; branched or unbranched, unsubstituted or mono- or poly-substituted; and
R³⁴ and R³⁵ each independently denote H; F; Cl; Br; CN; NH₂; C₁₋₈-alkyl; CF₃; OH; O—C₁₋₈-alkyl; OCF₃; or SCF₃.

Particularly preferably, R¹¹ and R¹², together with the nitrogen atom joining them as ring member, form one of the following groups:

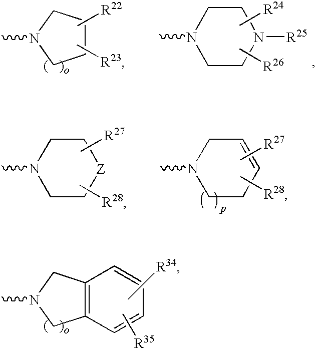

wherein
Z represents O or S;
o is 0, 1 or 2;
p is 0 or 1;
R²², R²³ and R²⁵ are each independently selected from the group consisting of H; C₁₋₈-alkyl, saturated or unsaturated; branched or unbranched; or one of the following groups:

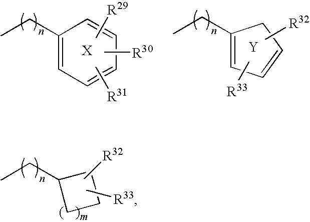

wherein
n is 0, 1, 2, 3 or 4;
m=0, 1, 2 or 3;
the ring X can contain a N atom;
the ring Y can contain up to 3 heteroatoms selected each independently from N, O and S;
R²⁹, R³⁰ and R³¹ each independently denote H; F; Cl; CN; CH₃; CF₃; OH; OCH₃; OCF₃; or SCF₃; or
R²⁹ and R³⁰, together with the carbon atoms joining them as ring members, form O—CH₂—O; or O—CH₂—CH₂—O;
R³² and R³³ each independently denote H; or CH₃; or
R²² and R²³, with the carbon atom(s) joining them as ring member(s), form a C₃₋₇-cycloalkyl group, saturated or unsaturated; or a fused phenyl or thienyl group, in each case unsubstituted or mono- or poly-substituted by F; Cl; CN; CH₃; CF₃; OH; OCH₃; OCF₃; or SCF₃;
R²⁴ and R²⁶ each independently denote H; C₁₋₈-alkyl, saturated or unsaturated; branched or unbranched; phenyl, unsubstituted or mono- or poly-substituted;
R²⁷ and R²⁸ each independently denote H; or CH₃; and
R³⁴ and R³⁵ each independently denote H; F; Cl; CN; CH₃; CF₃; OH; OCH₃; OCF₃; or SCF₃.

Particular preference is given to compounds in which R¹¹ and R¹², together with the nitrogen atom joining them as ring member, form the group

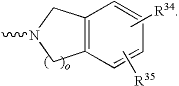

Particular preference is given to compounds of formula (1) wherein R¹ is selected from the group consisting of H; C₁₋₈-alkyl; C₃₋₇-cycloalkyl; C₁-alkyl-bridged C₃₋₇-cycloalkyl; phenyl, unsubstituted or mono- or di-substituted by substituents selected each independently from the group consisting of F, Cl, Br, CH₃, C₂H₅, isopropyl, OCH₃ and CF₃; thienyl, unsubstituted or mono- or di-substituted by CH₃; C₁₋₃-alkyl-bridged saturated, unsubstituted phenyl;
R² is selected from H; CH₃ or C₂H₅, but preferably denotes H; or
R¹ and R², together with the carbon atom joining them as ring member, form a saturated and unsubstituted C₃₋₇-cycloalkyl group optionally fused with phenyl;
R³, R⁴ and R⁵ each denote H, and R⁶ denotes H, —CH₃ or phenyl;
one of R⁷ or R⁹ is selected from the group consisting of H; CH₃; phenyl, unsubstituted; and the other represents H; or
R⁷ and R⁹, together with the carbon atoms joining them as ring members, form an unsubstituted C₃₋₇-cycloalkyl group, saturated or unsaturated, optionally fused with unsubstituted phenyl;
R⁸ and R¹⁰ each denote H;
R⁷ and R⁸; or R⁹ and R¹⁰; together with the carbon atoms joining them as ring members, form an unsubstituted C₃₋₇-cycloalkyl group, saturated or unsaturated, optionally fused with unsubstituted phenyl;
R¹¹ represents H; saturated or unsaturated, branched or unbranched C₁₋₈-alkyl; or benzyl; and preferably is selected from the group consisting of H; CH₃; C₂H₅; propyl; isopropyl; isopentyl; and benzyl;
R¹² represents saturated or unsaturated, branched or unbranched C₁₋₈-alkyl; saturated or unsaturated C₃₋₇-cycloalkyl optionally fused with phenyl, wherein the phenyl ring can be unsubstituted or mono- or di-substituted by CF₃; indazolyl; benzothiadiazolyl, in each case unsubstituted or monosubstituted by CH₃; phenyl, unsubsituted or mono- or di-substituted by substituents selected each independently selected from the group consisting of F; Cl; OCH₃; CH₃; CF₃; O—CH₂—O; benzyl; C₁₋₄-alkyl-bridged napththyl, imidazolyl, indolyl, pyrimidinyl, pyridyl, triazolyl, furanyl or thienyl, in each case unsubstituted or monosubstituted by CH₃, wherein the alkyl chain in each case can be saturated or unsaturated, branched or unbranched; C₁₋₄-alkyl-bridged C₃₋₇-cycloalkyl, saturated or unsaturated; C₁₋₄-alkyl-bridged piperidinyl, tetrahydrofuranyl, morpholinyl or diphenylmethyl; C₁₋₄-alkyl-bridged isoxazolyl, unsubstituted or mono- or di-substituted by substituents selected each independently from the group consisting of CH₃ and phenyl; C₁₋₄-alkyl-bridged phenyl, unsubstituted or mono- or di-substituted by substituents selected each independently from the group consisting of F, Cl, Br, CH₃, CF₃, OCH₃, O—CH₂—O, benzyl, wherein the alkyl chain in each case can be saturated or unsaturated, branched or unbranched; or
R¹¹ and R¹², together with the nitrogen atom joining them as ring member, form a piperazinyl, unsubstituted or mono- or di-substituted by saturated or unsaturated, branched or unbranched C₁₋₄-alkyl; C(═O)CH₃; C(═O)C₂H₅; C(═O)OCH₃; C(═O)OC₂H₅; C(═O)-furanyl; phenyl, unsubstituted or mono- or di-substituted by substituents selected each independently from the group consisting of F, Cl, Br, CH₃, CF₃, OCH₃ and O—CH₂—O; pyrimidinyl, unsubstituted; pyridyl, in each case unsubstituted or mono- or di-substituted by substituents selected each independently from the group consisting of F, Cl and CH₃; C₁₋₄-alkyl-bridged phenyl, unsubstituted or mono-, di- or tri-substituted by substituents selected each independently from the group consisting of F, Cl, Br, CH₃, tert-butyl, CF₃, OCH₃ and O—CH₂—O, wherein the alkyl chain in each case can be saturated or unsaturated, branched or unbranched; C₁₋₄-alkyl-bridged saturated or unsaturated C₃₋₇-cycloalkyl optionally fused with unsubstituted phenyl, wherein the alkyl chain in each case can be saturated or unsaturated, branched or unbranched; or
R¹¹ and R¹², together with the nitrogen atom joining them as ring member, form a piperidinyl, unsubstituted or mono- or di-substituted by C₁₋₄-alkyl, saturated or unsaturated, branched or unbranched; C(═O)OCH₃; C(═O)OC₂H₅; phenyl, unsubstituted or mono- or di-substituted by substituents selected each independently from the group consisting of F, Cl, CH₃, OCH₃ and CF₃; C₁₋₄-alkyl-bridged phenyl, unsubstituted, wherein the alkyl chain in each case can be saturated or unsaturated, branched or unbranched; C₁₋₄-alkyl-bridged pyridyl, unsubstituted or mono- or di-substituted by substituents selected each independently from the group consisting of CH₃, OCH₃ and CF₃, wherein the alkyl chain in each case can be saturated or unsaturated, branched or unbranched; and/or the piperidinyl can optionally be fused with cyclohexyl, phenyl or thienyl, in each case unsubstituted or mono- or di-substituted by CF₃; or
R¹¹ and R¹², together with the nitrogen atom joining them as ring member, form an azetidinyl, aziridinyl, tetrahydropyridinyl or dihydropyrrolyl group; or R¹¹ and R¹², together with the nitrogen atom joining them as ring member, form a pyrrolidinyl, unsubstituted or mono- or di-substituted by C₁₋₄-alkyl, saturated or unsaturated, branched or unbranched; CH₂OCH₃; phenyl, unsubstituted; pyridyl, unsubstituted or monosubstituted by substituents selected each independently from the group consisting of F, Cl, Br and CH₃; isothiazolyl; thiazolyl; C₁₋₄-alkyl-bridged pyridyl, unsubstituted or mono- or di-substituted by substituents selected each independently from the group consisting of CH₃, C₂H₅, F, Cl, Br, wherein the alkyl chain in each case can be saturated or unsaturated, branched or unbranched; C₁₋₄-alkyl-bridged phenyl, unsubstituted, wherein the alkyl chain in each case can be saturated or unsaturated, branched or unbranched; and/or the pyrrolidinyl can optionally be fused with cyclohexyl, phenyl or thienyl, in each case unsubstituted or mono- or di-substituted by F; CH₃; CF₃; or
R¹¹ and R¹², together with the nitrogen atom joining them as ring member, form a morpholinyl, unsubstituted or mono- or di-substituted by CH₃; or
R¹¹ and R¹², together with the nitrogen atom joining them as ring member, form a thiomorpholinyl group; and
one of R¹³ and R¹⁴; or R¹³ and R¹⁵; or R¹⁴ and R¹⁵ represents CH₃ and the other denotes H.

Most particular preference is given to compounds selected from the group consisting of:
1 4-oxo-4-(4-(thiophen-2-yl)-6,7-dihydrothieno[3,2-c]pyridin-5(4H)-yl)-N-(3-(trifluoromethyl)benzyl)butanamide;
2 4-oxo-4-(4-phenyl-6,7-dihydrothieno[3,2-c]pyridin-5(4H)-yl)-N-(3-(trifluoromethyl)benzyl)butanamide;
3 4-(4-(3-fluorophenyl)-6,7-dihydrothieno[3,2-c]pyridin-5(4H)-yl)-4-oxo-N-(3-(trifluoromethyl)benzyl)butanamide;
4 4-oxo-4-(4-m-tolyl-6,7-dihydrothieno[3,2-c]pyridin-5(4H)-yl)-N-(3-(trifluoromethyl)benzyl)butanamide;
5 4-oxo-4-(4-p-tolyl-6,7-dihydrothieno[3,2-c]pyridin-5(4H)-yl)-N-(3-(trifluoromethyl)benzyl)butanamide;
6 4-(4-(2-fluorophenyl)-6,7-dihydrothieno[3,2-c]pyridin-5(4H)-yl)-4-oxo-N-(3-(trifluoromethyl)benzyl)butanamide;
7 4-(4-(4-fluorophenyl)-6,7-dihydrothieno[3,2-c]pyridin-5(4H)-yl)-4-oxo-N-(3-(trifluoromethyl)benzyl)butanamide;
8 4-oxo-4-(4-o-tolyl-6,7-dihydrothieno[3,2-c]pyridin-5(4H)-yl)-N-(3-(trifluoromethyl)benzyl)butanamide;
9 4-oxo-4-(7-phenyl-4,5-dihydrothieno[2,3-c]pyridin-6(7H)-yl)-N-(3-(trifluoromethyl)benzyl)butanamide;
10 4-(2-methyl-4-phenyl-6,7-dihydrothieno[3,2-c]pyridin-5(4H)-yl)-4-oxo-N-(3-(trifluoromethyl)benzyl)butanamide;
11 N-(4-methylbenzyl)-4-oxo-4-(4-phenyl-6,7-dihydrothieno[3,2-c]pyridin-5(4H)-yl)butanamide;
12 N-benzyl-4-oxo-4-(4-phenyl-6,7-dihydrothieno[3,2-c]pyridin-5(4H)-yl)butanamide;
13 4-oxo-4-(4-phenyl-6,7-dihydrothieno[3,2-c]pyridin-5(4H)-yl)-N-(4-(trifluoromethyl)benzyl)butanamide;
14 N-(2-methoxybenzyl)-4-oxo-4-(4-phenyl-6,7-dihydrothieno[3,2-c]pyridin-5(4H)-yl)butanamide;
15 N-(3-methoxybenzyl)-4-oxo-4-(4-phenyl-6,7-dihydrothieno[3,2-c]pyridin-5(4H)-yl)butanamide;
16 N-(4-methoxybenzyl)-4-oxo-4-(4-phenyl-6,7-dihydrothieno[3,2-c]pyridin-5(4H)-yl)butanamide;
17 4-oxo-4-(4-phenyl-6,7-dihydrothieno[3,2-c]pyridin-5(4H)-yl)-N-(2-(trifluoromethyl)benzyl)butanamide;
18 N-(3-fluorobenzyl)-4-oxo-4-(4-phenyl-6,7-dihydrothieno[3,2-c]pyridin-5(4H)-yl)butanamide;
19 N-(3-methylbenzyl)-4-oxo-4-(4-phenyl-6,7-dihydrothieno[3,2-c]pyridin-5(4H)-yl)butanamide;
20 4-(3-methyl-4-phenyl-6,7-dihydrothieno[3,2-c]pyridin-5(4H)-yl)-4-oxo-N-(3-(trifluoromethyl)benzyl)butanamide;
21 4-(4-cyclohexyl-6,7-dihydrothieno[3,2-c]pyridin-5(4H)-yl)-4-oxo-N-(3-(trifluoromethyl)benzyl)butanamide;
22 4-(4-isopropyl-6,7-dihydrothieno[3,2-c]pyridin-5(4H)-yl)-4-oxo-N-(3-(trifluoromethyl)benzyl)butanamide;
23 4-(4-butyl-6,7-dihydrothieno[3,2-c]pyridin-5(4H)-yl)-4-oxo-N-(3-(trifluoromethyl)benzyl)butanamide;
24 N-methyl-4-oxo-4-(4-phenyl-6,7-dihydrothieno[3,2-c]pyridin-5(4H)-yl)-N-(3-(trifluoromethyl)benzyl)butanamide;
25 4-oxo-N-phenethyl-4-(4-phenyl-6,7-dihydrothieno[3,2-c]pyridin-5(4H)-yl)butanamide;
26 N-(2-methylbenzyl)-4-oxo-4-(4-phenyl-6,7-dihydrothieno[3,2-c]pyridin-5(4H)-yl)butanamide;
27 4-oxo-2-phenyl-4-(4-phenyl-6,7-dihydrothieno[3,2-c]pyridin-5(4H)-yl)-N-(3-(trifluoromethyl)benzyl)butanamide;
28 (R)-2-methyl-4-oxo-4-(4-phenyl-6,7-dihydrothieno[3,2-c]pyridin-5(4H)-yl)-N-(3-(trifluoromethyl)benzyl)butanamide;
29 4-oxo-3-phenyl-4-(4-phenyl-6,7-dihydrothieno[3,2-c]pyridin-5(4H)-yl)-N-(3-(trifluoromethyl)benzyl)butanamide;
30 4-(6,7-dihydrothieno[3,2-c]pyridin-5(4H)-yl)-4-oxo-3-phenyl-N-(3-(trifluoromethyl)benzyl)butanamide;
31 rac. (1S,2R)-2-(4-phenyl-4,5,6,7-tetrahydrothieno[3,2-c]pyridine-5-carbonyl)-N-(3-(trifluoromethyl)benzyl)cyclohexanecarboxamide;
32 rac. (1S)-2-(4-phenyl-4,5,6,7-tetrahydrothieno[3,2-c]pyridine-5-carbonyl)-N-(3-(trifluoromethyl)benzyl)cyclohexanecarboxamide;
33 rac. (1S,2R)-2-(4-phenyl-4,5,6,7-tetrahydrothieno[3,2-c]pyridine-5-carbonyl)-N-(3-(trifluoromethyl)benzyl)cyclopentanecarboxamide;
34 (R)-3-methyl-4-oxo-4-(4-phenyl-6,7-dihydrothieno[3,2-c]pyridin-5(4H)-yl)-N-(3-(trifluoromethyl)benzyl)butanamide;
35 (−)-4-oxo-4-(4-phenyl-6,7-dihydrothieno[3,2-c]pyridin-5(4H)-yl)-N-(3-(trifluoromethyl)benzyl)butanamide;
36 (+)-4-oxo-4-(4-phenyl-6,7-dihydrothieno[3,2-c]pyridin-5(4H)-yl)-N-(3-(trifluoromethyl)benzyl)butanamide;
37 4-(6,7-dihydrothieno[3,2-c]pyridin-5(4H)-yl)-4-oxo-2-phenyl-N-(3-(trifluoromethyl)benzyl)butanamide;
38 N-(3,5-bis(trifluoromethyl)benzyl)-4-oxo-4-(4-phenyl-6,7-dihydrothieno[3,2-c]-pyridin-5(4H)-yl)butanamide;
39 N-(2-fluoro-5-(trifluoromethyl)benzyl)-4-oxo-4-(4-phenyl-6,7-dihydrothieno-[3,2-c]pyridin-5(4H)-yl)butanamide;
40 N-(2-fluoro-3-(trifluoromethyl)benzyl)-4-oxo-4-(4-phenyl-6,7-dihydrothieno-[3,2-c]pyridin-5(4H)-yl)butanamide;
41 N-(3-fluoro-5-(trifluoromethyl)benzyl)-4-oxo-4-(4-phenyl-6,7-dihydrothieno-[3,2-c]pyridin-5(4H)-yl)butanamide;
42 rac. (1S,2R)-2-(4-phenyl-4,5,6,7-tetrahydrothieno[3,2-c]pyridine-5-carbonyl)-N-(3-(trifluoromethyl)benzyl)cyclopropanecarboxamide;
43 N-(3,4-difluorobenzyl)-4-oxo-4-(4-phenyl-6,7-dihydrothieno[3,2-c]pyridin-5(4H)-yl)butanamide;
44 4-oxo-4-(5-phenyl-4,5-dihydrothieno[2,3-c]pyridin-6(7H)-yl)-N-(3-(trifluoromethyl)benzyl)butanamide;
45 4-(1-methyl-4-phenyl-6,7-dihydrothieno[3,4-c]pyridin-5(4H)-yl)-4-oxo-N-(3-(trifluoromethyl)benzyl)butanamide;
46 N-(4-methoxyphenyl)-4-oxo-4-(4-p-tolyl-6,7-dihydrothieno[3,2-c]pyridin-5(4H)-yl)butanamide;
47 N-(1-methyl-1H-indazol-6-yl)-4-oxo-4-(4-p-tolyl-6,7-dihydrothieno[3,2-c]pyridin-5(4H)-yl)butanamide;
48 N-benzyl-4-oxo-4-(4-p-tolyl-6,7-dihydrothieno[3,2-c]pyridin-5(4H)-yl)butanamide;
49 4-oxo-N-phenethyl-4-(4-p-tolyl-6,7-dihydrothieno[3,2-c]pyridin-5(4H)-yl)butanamide;
50 4-oxo-N-(pyridin-4-ylmethyl)-4-(4-p-tolyl-6,7-dihydrothieno[3,2-c]pyridin-5(4H)-yl)butanamide;
51 4-oxo-N-(3-phenylpropyl)-4-(4-p-tolyl-6,7-dihydrothieno[3,2-c]pyridin-5(4H)-yl)butanamide;
52 N-(benzo[c][1,2,5]thiadiazol-4-yl)-4-oxo-4-(4-m-tolyl-6,7-dihydrothieno[3,2-c]-pyridin-5(4H)-yl)butanamide;
53 N-(1-methyl-1H-indazol-6-yl)-4-oxo-4-(4-m-tolyl-6,7-dihydrothieno[3,2-c]-pyridin-5(4H)-yl)butanamide;
54 4-oxo-N-(pyridin-2-ylmethyl)-4-(4-m-tolyl-6,7-dihydrothieno[3,2-c]pyridin-5(4H)-yl)butanamide;
55 1-(4-methylpiperazin-1-yl)-4-(4-m-tolyl-6,7-dihydrothieno[3,2-c]pyridin-5(4H)-yl)butane-1,4-dione;
56 3-methyl-4-oxo-4-(4-phenyl-6,7-dihydrothieno[3,2-c]pyridin-5(4H)-yl)-N-(pyridin-2-ylmethyl)butanamide;
57 4-oxo-4-(4-phenyl-6,7-dihydrothieno[3,2-c]pyridin-5(4H)-yl)-N-(thiophen-2-ylmethyl)butanamide;
58 N-(2-chlorophenyl)-4-(4-(4-fluorophenyl)-6,7-dihydrothieno[3,2-c]pyridin-5(4H)-yl)-4-oxobutanamide;
59 4-(4-(4-chlorophenyl)-6,7-dihydrothieno[3,2-c]pyridin-5(4H)-yl)-N-(furan-2-ylmethyl)-4-oxobutanamide;
60 4-(4-(4-chlorophenyl)-6,7-dihydrothieno[3,2-c]pyridin-5(4H)-yl)-4-oxo-N-propylbutanamide;
61 N-(2-chlorobenzyl)-4-oxo-4-(4-m-tolyl-6,7-dihydrothieno[3,2-c]pyridin-5(4H)-yl)butanamide;
62 N-(2,4-dichlorobenzyl)-4-(4-(3-fluorophenyl)-6,7-dihydrothieno[3,2-c]pyridin-5(4H)-yl)-4-oxobutanamide;
63 N-(4-fluorobenzyl)-4-(4-(3-fluorophenyl)-6,7-dihydrothieno[3,2-c]pyridin-5(4H)-yl)-4-oxobutanamide;
64 N-(3,4-dichlorobenzyl)-3-methyl-4-oxo-4-(4-phenyl-6,7-dihydrothieno[3,2-c]-pyridin-5(4H)-yl)butanamide;
65 N-(2,5-difluorobenzyl)-3-methyl-4-oxo-4-(4-phenyl-6,7-dihydrothieno[3,2-c]-pyridin-5(4H)-yl)butanamide;
66 3-methyl-4-oxo-4-(4-phenyl-6,7-dihydrothieno[3,2-c]pyridin-5(4H)-yl)-N-(3-(trifluoromethyl)benzyl)butanamide;
67 1-(4-(4-chlorophenyl)-6,7-dihydrothieno[3,2-c]pyridin-5(4H)-yl)-4-morpholino-butane-1,4-dione;
68 1-(4-(4-chlorophenyl)-6,7-dihydrothieno[3,2-c]pyridin-5(4H)-yl)-4-(4-(4-fluorophenyl)piperazin-1-yl)butane-1,4-dione;
69 2-methyl-N-(2-(5-methyl-1H-pyrazol-1-yl)ethyl)-4-oxo-4-(4-phenyl-6,7-dihydro-thieno[3,2-c]pyridin-5(4H)-yl)butanamide;
70 N-(naphthalen-1-ylmethyl)-4-oxo-4-(4-phenyl-6,7-dihydrothieno[3,2-c]pyridin-5(4H)-yl)butanamide;
71 4-(4-(4-fluorophenyl)-6,7-dihydrothieno[3,2-c]pyridin-5(4H)-yl)-N-(4-methyl-benzyl)-4-oxobutanamide;
72 N-(benzo[d][1,3]dioxol-5-ylmethyl)-4-(4-(4-fluorophenyl)-6,7-dihydrothieno-[3,2-c]pyridin-5(4H)-yl)-4-oxobutanamide;
73 4-(4-(4-fluorophenyl)-6,7-dihydrothieno[3,2-c]pyridin-5(4H)-yl)-4-oxo-N-(2-(trifluoromethyl)benzyl)butanamide;
74 4-(4-(4-fluorophenyl)-6,7-dihydrothieno[3,2-c]pyridin-5(4H)-yl)-N-(naphthalen-1-ylmethyl)-4-oxobutanamide;
75 4-(4-(4-chlorophenyl)-6,7-dihydrothieno[3,2-c]pyridin-5(4H)-yl)-N-(3-fluorobenzyl)-4-oxobutanamide;
76 2-methyl-N-(1-methyl-1H-indazol-6-yl)-4-oxo-4-(4-phenyl-6,7-dihydrothieno-[3,2-c]pyridin-5(4H)-yl)butanamide;
77 N-(4-methoxybenzyl)-2-methyl-4-oxo-4-(4-phenyl-6,7-dihydrothieno[3,2-c]-pyridin-5(4H)-yl)butanamide;
78 2-methyl-1-(4-methylpiperazin-1-yl)-4-(4-phenyl-6,7-dihydrothieno[3,2-c]-pyridin-5(4H)-yl)butane-1,4-dione;
79 N-(2-(1H-indo1-3-yl)ethyl)-2-methyl-4-oxo-4-(4-phenyl-6,7-dihydrothieno[3,2-c]-pyridin-5(4H)-yl)butanamide;
80 N-(2-fluorobenzyl)-2-methyl-4-oxo-4-(4-phenyl-6,7-dihydrothieno[3,2-c]pyridin-5(4H)-yl)butanamide;
81 2-methyl-4-oxo-4-(4-phenyl-6,7-dihydrothieno[3,2-c]pyridin-5(4H)-yl)-N-(2-(trifluoromethyl)benzyl)butanamide;
82 4-oxo-N-(2-(piperidin-1-yl)ethyl)-4-(4-p-tolyl-6,7-dihydrothieno[3,2-c]pyridin-5(4H)-yl)butanamide;
83 4-oxo-N-((tetrahydrofuran-2-yl)methyl)-4-(4-p-tolyl-6,7-dihydrothieno[3,2-c]-pyridin-5(4H)-yl)butanamide;
84 N-(4-chlorobenzyl)-4-oxo-4-(4-p-tolyl-6,7-dihydrothieno[3,2-c]pyridin-5(4H)-yl)butanamide;
85 N-(2,3-dichlorobenzyl)-4-oxo-4-(4-p-tolyl-6,7-dihydrothieno[3,2-c]pyridin-5(4H)-yl)butanamide;
86 4-oxo-N-(2-(thiophen-2-yl)ethyl)-4-(4-m-tolyl-6,7-dihydrothieno[3,2-c]pyridin-5(4H)-yl)butanamide;
87 N-(cyclohexylmethyl)-4-oxo-4-(4-m-tolyl-6,7-dihydrothieno[3,2-c]pyridin-5(4H)-yl)butanamide;
88 N-(3-chlorophenethyl)-4-oxo-4-(4-m-tolyl-6,7-dihydrothieno[3,2-c]pyridin-5(4H)-yl)butanamide;
89 N-(3,3-diphenylpropyl)-4-oxo-4-(4-m-tolyl-6,7-dihydrothieno[3,2-c]pyridin-5(4H)-yl)butanamide;
90 4-(4-(3-fluorophenyl)-6,7-dihydrothieno[3,2-c]pyridin-5(4H)-yl)-N-(3-morpholinopropyl)-4-oxobutanamide;
91 4-(4-(3-fluorophenyl)-6,7-dihydrothieno[3,2-c]pyridin-5(4H)-yl)-4-oxo-N-(pyridin-3-ylmethyl)butanamide;
92 4-(4-(3-fluorophenyl)-6,7-dihydrothieno[3,2-c]pyridin-5(4H)-yl)-N-(3-methyl-benzyl)-4-oxobutanamide;
93 4-(4-(3-fluorophenyl)-6,7-dihydrothieno[3,2-c]pyridin-5(4H)-yl)-N-(4-methylphenethyl)-4-oxobutanamide;
94 3-methyl-4-oxo-4-(4-phenyl-6,7-dihydrothieno[3,2-c]pyridin-5(4H)-yl)-N-(4-phenylbutyl)butanamide;
95 N-(biphenyl-4-ylmethyl)-4-oxo-4-(4-m-tolyl-6,7-dihydrothieno[3,2-c]pyridin-5(4H)-yl)butanamide;
96 4-(4-(3-fluorophenyl)-6,7-dihydrothieno[3,2-c]pyridin-5(4H)-yl)-N-(2-methoxybenzyl)-4-oxobutanamide;
97 N-(4-chlorophenethyl)-4-(4-(3-fluorophenyl)-6,7-dihydrothieno[3,2-c]pyridin-5(4H)-yl)-4-oxobutanamide;
98 4-(4-(3-fluorophenyl)-6,7-dihydrothieno[3,2-c]pyridin-5(4H)-yl)-N-((5-methyl-3-phenylisoxazol-4-yl)methyl)-4-oxobutanamide;
99 N-(2,6-difluorobenzyl)-3-methyl-4-oxo-4-(4-phenyl-6,7-dihydrothieno[3,2-c]-pyridin-5(4H)-yl)butanamide;
100 N-(3,5-difluorobenzyl)-3-methyl-4-oxo-4-(4-phenyl-6,7-dihydrothieno[3,2-c]-pyridin-5(4H)-yl)butanamide;
101 N-(3-chlorobenzyl)-3-methyl-4-oxo-4-(4-phenyl-6,7-dihydrothieno[3,2-c]pyridin-5(4H)-yl)butanamide;
102 N-(3,5-dimethoxyphenethyl)-3-methyl-4-oxo-4-(4-phenyl-6,7-dihydrothieno-[3,2-c]pyridin-5(4H)-yl)butanamide;
103 N-(3,4-difluorobenzyl)-4-oxo-4-(4-phenyl-6,7-dihydrothieno[3,2-c]pyridin-5(4H)-yl)butanamide;
104 N-(2,4-dichlorophenethyl)-4-oxo-4-(4-phenyl-6,7-dihydrothieno[3,2-c]pyridin-5(4H)-yl)butanamide;
105 N-(2-(1H-1,2,4-triazol-1-yl)ethyl)-4-oxo-4-(4-phenyl-6,7-dihydrothieno[3,2-c]-pyridin-5(4H)-yl)butanamide;
106 4-(4-(4-fluorophenyl)-6,7-dihydrothieno[3,2-c]pyridin-5(4H)-yl)-N-(2-methyl-benzyl)-4-oxobutanamide;
107 N-(4-fluorophenethyl)-4-(4-(4-fluorophenyl)-6,7-dihydrothieno[3,2-c]pyridin-5(4H)-yl)-4-oxobutanamide;
108 4-(4-(4-fluorophenyl)-6,7-dihydrothieno[3,2-c]pyridin-5(4H)-yl)-N-((5-methyl-isoxazol-3-yl)methyl)-4-oxobutanamide;
109 N-(2-chlorophenethyl)-4-(4-(4-fluorophenyl)-6,7-dihydrothieno[3,2-c]pyridin-5(4H)-yl)-4-oxobutanamide;
110 4-(4-(4-chlorophenyl)-6,7-dihydrothieno[3,2-c]pyridin-5(4H)-yl)-N-(2-cyclo-hexenylethyl)-4-oxobutanamide;
111 4-(4-(4-chlorophenyl)-6,7-dihydrothieno[3,2-c]pyridin-5(4H)-yl)-N-(3,5-dimethoxybenzyl)-4-oxobutanamide;
112 4-(4-(4-chlorophenyl)-6,7-dihydrothieno[3,2-c]pyridin-5(4H)-yl)-N-(3,4-dichlorophenethyl)-4-oxobutanamide;
113 4-(4-(4-chlorophenyl)-6,7-dihydrothieno[3,2-c]pyridin-5(4H)-yl)-N-(3-fluorophenethyl)-4-oxobutanamide;
114 1-(3-phenylpiperidin-1-yl)-4-(4-p-tolyl-6,7-dihydrothieno[3,2-c]pyridin-5(4H)-yl)butane-1,4-dione;
115 1-(4-benzylpiperazin-1-yl)-4-(4-p-tolyl-6,7-dihydrothieno[3,2-c]pyridin-5(4H)-yl)butane-1,4-dione;
116 1-(4-(4-methoxyphenyl)piperazin-1-yl)-4-(4-p-tolyl-6,7-dihydrothieno[3,2-c]-pyridin-5(4H)-yl)butane-1,4-dione;
117 1-(2-benzylpiperidin-1-yl)-4-(4-m-tolyl-6,7-dihydrothieno[3,2-c]pyridin-5(4H)-yl)butane-1,4-dione;
118 1-(4-(2-fluorophenyl)piperazin-1-yl)-4-(4-m-tolyl-6,7-dihydrothieno[3,2-c]-pyridin-5(4H)-yl)butane-1,4-dione;
119 1-(4-(cyclohexylmethyl)piperazin-1-yl)-4-(4-m-tolyl-6,7-dihydrothieno[3,2-c]-pyridin-5(4H)-yl)butane-1,4-dione;
120 N-(3-(1H-imidazol-1-yl)propyl)-4-(4-(3-fluorophenyl)-6,7-dihydrothieno[3,2-c]-pyridin-5(4H)-yl)-4-oxobutanamide;
121 1-(4-(3-fluorophenyl)-6,7-dihydrothieno[3,2-c]pyridin-5(4H)-yl)-4-(3-phenyl-pyrrolidin-1-yl)butane-1,4-dione;
122 1-(4-(3-fluorophenyl)-6,7-dihydrothieno[3,2-c]pyridin-5(4H)-yl)-4-(4-(furan-2-carbonyl)piperazin-1-yl)butane-1,4-dione;
123 N-(3-(3,4-dihydroquinolin-1(2H)-yl)propyl)-3-methyl-4-oxo-4-(4-phenyl-6,7-dihydrothieno[3,2-c]pyridin-5(4H)-yl)butanamide;
124 2-methyl-1-(4-phenyl-6,7-dihydrothieno[3,2-c]pyridin-5(4H)-yl)-4-(piperidin-1-yl)butane-1,4-dione;
125 4-(4-(2-methoxyphenyl)piperazin-1-yl)-2-methyl-1-(4-phenyl-6,7-dihydrothieno-[3,2-c]pyridin-5(4H)-yl)butane-1,4-dione;
126 4-oxo-4-(4-phenyl-6,7-dihydrothieno[3,2-c]pyridin-5(4H)-yl)-N-(2-(pyrazin-2-yl)ethyl)butanamide;
127 N,N-diethyl-4-oxo-4-(4-phenyl-6,7-dihydrothieno[3,2-c]pyridin-5(4H)-yl)-butanamide;
128 1-(4-phenyl-6,7-dihydrothieno[3,2-c]pyridin-5(4H)-yl)-4-(4-(pyridin-2-yl)piperazin-1-yl)butane-1,4-dione;
129 1-(4-(4-fluorophenyl)-6,7-dihydrothieno[3,2-c]pyridin-5(4H)-yl)-4-(4-(3-methoxyphenyl)piperazin-1-yl)butane-1,4-dione;
130 1-(4-isopropylpiperazin-1-yl)-4-(4-p-tolyl-6,7-dihydrothieno[3,2-c]pyridin-5(4H)-yl)butane-1,4-dione;
131 1-(4-p-tolyl-6,7-dihydrothieno[3,2-c]pyridin-5(4H)-yl)-4-(4-(3-(trifluoromethyl)-phenyl)piperazin-1-yl)butane-1,4-dione;
132 1-(4-phenylpiperazin-1-yl)-4-(4-m-tolyl-6,7-dihydrothieno[3,2-c]pyridin-5(4H)-yl)butane-1,4-dione;
133 1-(3,5-dimethylpiperidin-1-yl)-4-(4-m-tolyl-6,7-dihydrothieno[3,2-c]pyridin-5(4H)-yl)butane-1,4-dione;
134 1-(5,6-dihydropyridin-1(2H)-yl)-4-(4-(3-fluorophenyl)-6,7-dihydrothieno[3,2-c]-pyridin-5(4H)-yl)butane-1,4-dione;
135 1-(4-(3-fluorophenyl)-6,7-dihydrothieno[3,2-c]pyridin-5(4H)-yl)-4-(4-(pyrimidin-2-yl)piperazin-1-yl)butane-1,4-dione;
136 1-(4-(3-fluorophenyl)-6,7-dihydrothieno[3,2-c]pyridin-5(4H)-yl)-4-(3-methyl-piperidin-1-yl)butane-1,4-dione;
137 N-ethyl-4-(4-(3-fluorophenyl)-6,7-dihydrothieno[3,2-c]pyridin-5(4H)-yl)-4-oxo-N-(pyridin-4-ylmethyl)butanamide;
138 4-(4-acetylpiperazin-1-yl)-2-methyl-1-(4-phenyl-6,7-dihydrothieno[3,2-c]pyridin-5(4H)-yl)butane-1,4-dione;
139 4-(2,6-dimethylmorpholino)-2-methyl-1-(4-phenyl-6,7-dihydrothieno[3,2-c]-pyridin-5(4H)-yl)butane-1,4-dione;
140 4-(2,5-dihydro-1H-pyrrol-1-yl)-2-methyl-1-(4-phenyl-6,7-dihydrothieno[3,2-c]-pyridin-5(4H)-yl)butane-1,4-dione;
141 N-(2-methoxyphenethyl)-4-oxo-4-(4-phenyl-6,7-dihydrothieno[3,2-c]pyridin-5(4H)-yl)butanamide;
142 4-(4-(4-fluorophenyl)-6,7-dihydrothieno[3,2-c]pyridin-5(4H)-yl)-N-isopentyl-4-oxobutanamide;
143 N-(2,4-dimethoxybenzyl)-4-(4-(4-fluorophenyl)-6,7-dihydrothieno[3,2-c]pyridin-5(4H)-yl)-4-oxobutanamide;
144 4-(4-(4-chlorophenyl)-6,7-dihydrothieno[3,2-c]pyridin-5(4H)-yl)-N-((5-methyl-furan-2-yl)methyl)-4-oxobutanamide;
145 4-(4-(4-chlorophenyl)-6,7-dihydrothieno[3,2-c]pyridin-5(4H)-yl)-4-oxo-N-pentylbutanamide;
146 1-(4-benzylpiperidin-1-yl)-4-(4-(4-chlorophenyl)-6,7-dihydrothieno[3,2-c]-pyridin-5(4H)-yl)butane-1,4-dione;
147 1-(4-(4-chlorophenyl)-6,7-dihydrothieno[3,2-c]pyridin-5(4H)-yl)-4-(4-methyl-piperidin-1-yl)butane-1,4-dione;
148 1-(azetidin-1-yl)-4-(4-(4-chlorophenyl)-6,7-dihydrothieno[3,2-c]pyridin-5(4H)-yl)butane-1,4-dione;
149 1-(3,4-dihydroisoquinolin-2(1H)-yl)-2-methyl-4-(4-phenyl-6,7-dihydrothieno-[3,2-c]pyridin-5(4H)-yl)butane-1,4-dione;
150 1-(4-(benzo[d][1,3]dioxol-5-ylmethyl)piperazin-1-yl)-2-methyl-4-(4-phenyl-6,7-dihydrothieno[3,2-c]pyridin-5(4H)-yl)butane-1,4-dione;
151 N-benzyl-N-ethyl-4-oxo-4-(4-p-tolyl-6,7-dihydrothieno[3,2-c]pyridin-5(4H)-yl)butanamide;
152 N-methyl-4-oxo-N-phenethyl-4-(4-p-tolyl-6,7-dihydrothieno[3,2-c]pyridin-5(4H)-yl)butanamide;
153 ethyl 1-(4-oxo-4-(4-p-tolyl-6,7-dihydrothieno[3,2-c]pyridin-5(4H)-yl)butanoyl)piperidine-4-carboxylate;
154 (E)-1-(4-(3-phenylprop-2-enyl)-piperazin-1-yl)-4-(4-p-tolyl-6,7-dihydrothieno-[3,2-c]pyridin-5(4H)-yl)butane-1,4-dione;
155 1-(2-(thiazol-2-yl)pyrrolidin-1-yl)-4-(4-p-tolyl-6,7-dihydrothieno[3,2-c]pyridin-5(4H)-yl)butane-1,4-dione;
156 N-cyclohexyl-N-ethyl-4-oxo-4-(4-m-tolyl-6,7-dihydrothieno[3,2-c]pyridin-5(4H)-yl)butanamide;
157 N-ethyl-N-isopropyl-4-oxo-4-(4-m-tolyl-6,7-dihydrothieno[3,2-c]pyridin-5(4H)-yl)butanamide;
158 1-(pyrrolidin-1-yl)-4-(4-m-tolyl-6,7-dihydrothieno[3,2-c]pyridin-5(4H)-yl)butane-1,4-dione;
159 1-(4-(pyridin-4-yl)piperazin-1-yl)-4-(4-m-tolyl-6,7-dihydrothieno[3,2-c]pyridin-5(4H)-yl)butane-1,4-dione;
160 1-(2-(pyridin-2-ylmethyl)pyrrolidin-1-yl)-4-(4-m-tolyl-6,7-dihydrothieno[3,2-c]-pyridin-5(4H)-yl)butane-1,4-dione;
161 ethyl 1-(4-(4-(4-fluorophenyl)-6,7-dihydrothieno[3,2-c]pyridin-5(4H)-yl)-4-oxo-butanoyl)piperidine-3-carboxylate;
162 1-(4-(4-fluorophenyl)-6,7-dihydrothieno[3,2-c]pyridin-5(4H)-yl)-4-(4-(4-(trifluoro-methyl)phenyl)piperazin-1-yl)butane-1,4-dione;
163 1-(2-(5-bromopyridin-3-yl)pyrrolidin-1-yl)-4-(4-(4-fluorophenyl)-6,7-dihydro-thieno[3,2-c]pyridin-5(4H)-yl)butane-1,4-dione;
164 1-(4-ethylpiperazin-1-yl)-4-(4-phenyl-6,7-dihydrothieno[3,2-c]pyridin-5(4H)-yl)butane-1,4-dione;
165 1-(2-ethylpiperidin-1-yl)-4-(4-phenyl-6,7-dihydrothieno[3,2-c]pyridin-5(4H)-yl)butane-1,4-dione;
166 N,N-dibenzyl-4-oxo-4-(4-phenyl-6,7-dihydrothieno[3,2-c]pyridin-5(4H)-yl)butanamide;
167 N,N-diisobutyl-4-oxo-4-(4-phenyl-6,7-dihydrothieno[3,2-c]pyridin-5(4H)-yl)butanamide;
168 1-(3,4-dihydroquinolin-1(2H)-yl)-4-(4-(4-fluorophenyl)-6,7-dihydrothieno[3,2-c]-pyridin-5(4H)-yl)butane-1,4-dione;
169 1-(4-(3,4-dichlorobenzyl)piperazin-1-yl)-4-(4-p-tolyl-6,7-dihydrothieno[3,2-c]-pyridin-5(4H)-yl)butane-1,4-dione;
170 1-(4-p-tolyl-6,7-dihydrothieno[3,2-c]pyridin-5(4H)-yl)-4-(4-(2,4,6-trimethyl-benzyl)piperazin-1-yl)butane-1,4-dione;
171 1-(4-(4-bromobenzyl)piperazin-1-yl)-4-(4-m-tolyl-6,7-dihydrothieno[3,2-c]-pyridin-5(4H)-yl)butane-1,4-dione;
172 1-(4-(4-chlorobenzyl)piperazin-1-yl)-4-(4-m-tolyl-6,7-dihydrothieno[3,2-c]-pyridin-5(4H)-yl)butane-1,4-dione;
173 1-(2-((4,6-dimethylpyridin-2-yl)methyl)pyrrolidin-1-yl)-4-(4-(3-fluorophenyl)-6,7-dihydrothieno[3,2-c]pyridin-5(4H)-yl)butane-1,4-dione;
174 1-(4-(3-fluorophenyl)-6,7-dihydrothieno[3,2-c]pyridin-5(4H)-yl)-4-(2-((6-methyl-pyridin-2-yl)methyl)pyrrolidin-1-yl)butane-1,4-dione;
175 1-(2-((5-ethylpyridin-2-yl)methyl)pyrrolidin-1-yl)-4-(4-(3-fluorophenyl)-6,7-dihydrothieno[3,2-c]pyridin-5(4H)-yl)butane-1,4-dione;
176 1-(4-(3-fluorophenyl)-6,7-dihydrothieno[3,2-c]pyridin-5(4H)-yl)-4-(2-(6-methoxypyridin-3-yl)piperidin-1-yl)butane-1,4-dione;
177 4-(4-(3-fluorophenyl)-6,7-dihydrothieno[3,2-c]pyridin-5(4H)-yl)-N-methyl-4-oxo-N-(pyridin-3-ylmethyl)butanamide;
178 N-(2,2-diphenylethyl)-3-methyl-4-oxo-4-(4-phenyl-6,7-dihydrothieno[3,2-c]-pyridin-5(4H)-yl)butanamide;
179 N-(cyclopropylmethyl)-4-oxo-4-(4-phenyl-6,7-dihydrothieno[3,2-c]pyridin-5(4H)-yl)butanamide;
180 1-(4-(3-fluorophenyl)-6,7-dihydrothieno[3,2-c]pyridin-5(4H)-yl)-4-(4-(4-methyl-benzyl)piperazin-1-yl)butane-1,4-dione;
181 2-methyl-1-(4-phenyl-6,7-dihydrothieno[3,2-c]pyridin-5(4H)-yl)-4-(2-(pyridin-2-ylmethyl)piperidin-1-yl)butane-1,4-dione;
182 4-(4-(3,5-dichloropyridin-4-yl)piperazin-1-yl)-2-methyl-1-(4-phenyl-6,7-dihydro-thieno[3,2-c]pyridin-5(4H)-yl)butane-1,4-dione;
183 1-(2-((4,6-dimethylpyridin-2-yl)methyl)piperidin-1-yl)-4-(4-phenyl-6,7-dihydro-thieno[3,2-c]pyridin-5(4H)-yl)butane-1,4-dione;
184 N-(4-fluorobenzyl)-N-methyl-4-oxo-4-(4-phenyl-6,7-dihydrothieno[3,2-c]pyridin-5(4H)-yl)butanamide;
185 1-(4-(3-fluorophenyl)-6,7-dihydrothieno[3,2-c]pyridin-5(4H)-yl)-4-(2-(pyridin-2-yl)pyrrolidin-1-yl)butane-1,4-dione;
186 4-(4-(4-methoxybenzyl)piperazin-1-yl)-2-methyl-1-(4-phenyl-6,7-dihydrothieno-[3,2-c]pyridin-5(4H)-yl)butane-1,4-dione;
187 2-methyl-1-(4-phenyl-6,7-dihydrothieno[3,2-c]pyridin-5(4H)-yl)-4-(3-(pyridin-3-yl)pyrrolidin-1-yl)butane-1,4-dione;
188 1-(4-(2-fluorobenzyl)piperazin-1-yl)-4-(4-phenyl-6,7-dihydrothieno[3,2-c]-pyridin-5(4H)-yl)butane-1,4-dione;
189 N-methyl-4-oxo-4-(4-phenyl-6,7-dihydrothieno[3,2-c]pyridin-5(4H)-yl)-N-(2-(pyridin-4-yl)ethyl)butanamide;
190 N-butyl-N-ethyl-3-methyl-4-oxo-4-(4-phenyl-6,7-dihydrothieno[3,2-c]pyridin-5(4H)-yl)butanamide;
191 N,3-dimethyl-4-oxo-4-(4-phenyl-6,7-dihydrothieno[3,2-c]pyridin-5(4H)-yl)-N-propylbutanamide;
192 4-((S)-2-(methoxymethyl)pyrrolidin-1-yl)-2-methyl-1-(4-phenyl-6,7-dihydro-thieno[3,2-c]pyridin-5(4H)-yl)butane-1,4-dione;
193 4-(4-(5-chloro-2-methylphenyl)piperazin-1-yl)-2-methyl-1-(4-phenyl-6,7-dihydrothieno[3,2-c]pyridin-5(4H)-yl)butane-1,4-dione;
194 N-(furan-2-ylmethyl)-N-methyl-4-oxo-4-(4-phenyl-6,7-dihydrothieno[3,2-c]-pyridin-5(4H)-yl)butanamide;
195 1-((4aR,8aS)-octahydroisoquinolin-2(1H)-yl)-4-(4-phenyl-6,7-dihydrothieno-[3,2-c]pyridin-5(4H)-yl)butane-1,4-dione;
196 1-(4-phenyl-6,7-dihydrothieno[3,2-c]pyridin-5(4H)-yl)-4-thiomorpholinobutane-1,4-dione;
197 1-(4-phenyl-6,7-dihydrothieno[3,2-c]pyridin-5(4H)-yl)-4-(2-(pyridin-3-yl)-pyrrolidin-1-yl)butane-1,4-dione;
198 N-benzyl-4-(4-(4-fluorophenyl)-6,7-dihydrothieno[3,2-c]pyridin-5(4H)-yl)-N-isopropyl-4-oxobutanamide;
199 N-benzyl-4-(4-(4-fluorophenyl)-6,7-dihydrothieno[3,2-c]pyridin-5(4H)-yl)-N-methyl-4-oxobutanamide;
200 N-(3,4-dimethoxyphenethyl)-4-(4-(4-fluorophenyl)-6,7-dihydrothieno[3,2-c]-pyridin-5(4H)-yl)-N-methyl-4-oxobutanamide;
201 1-(4-(4-fluorophenyl)-6,7-dihydrothieno[3,2-c]pyridin-5(4H)-yl)-4-(4-methyl-2-phenylpiperazin-1-yl)butane-1,4-dione;
202 1-(4-(4-chlorophenyl)-6,7-dihydrothieno[3,2-c]pyridin-5(4H)-yl)-4-(4-phenyl-piperidin-1-yl)butane-1,4-dione;
203 4-(4-(4-chlorophenyl)-6,7-dihydrothieno[3,2-c]pyridin-5(4H)-yl)-4-oxo-N-(2-(pyridin-2-yl)ethyl)butanamide;
204 N-benzyl-2-methyl-4-oxo-N-phenethyl-4-(4-phenyl-6,7-dihydrothieno[3,2-c]-pyridin-5(4H)-yl)butanamide;
205 1-(4-(4-fluorophenyl)-6,7-dihydrothieno[3,2-c]pyridin-5(4H)-yl)-4-(2-((6-methyl-pyridin-2-yl)methyl)piperidin-1-yl)butane-1,4-dione;
206 1-(4-(4-fluorophenyl)-6,7-dihydrothieno[3,2-c]pyridin-5(4H)-yl)-4-(4-(3-methyl-benzyl)piperazin-1-yl)butane-1,4-dione;
207 1-(4-(4-chlorophenyl)-6,7-dihydrothieno[3,2-c]pyridin-5(4H)-yl)-4-(2-(pyridin-4-ylmethyl)pipendin-1-yl)butane-1,4-dione;
208 1-(4-(4-chlorophenyl)-6,7-dihydrothieno[3,2-c]pyridin-5(4H)-yl)-4-(2-((5-ethyl-pyridin-2-yl)methyl)piperidin-1-yl)butane-1,4-dione;
209 1-(4-(4-chlorophenyl)-6,7-dihydrothieno[3,2-c]pyridin-5(4H)-yl)-4-(2-((3-methyl-pyridin-2-yl)methyl)piperidin-1-yl)butane-1,4-dione;
210 1-(4-(4-chlorophenyl)-6,7-dihydrothieno[3,2-c]pyridin-5(4H)-yl)-4-(4-(3-chloro-phenyl)piperazin-1-yl)butane-1,4-dione;
211 1-(4-(4-dhlorophenyl)-6,7-dihydrothieno[3,2-c]pyridin-5(4H)-yl)-4-(4-(2,3-dimethylphenyl)piperazin-1-yl)butane-1,4-dione;
212 1-(4-(4-chlorophenyl)-6,7-dihydrothieno[3,2-c]pyridin-5(4H)-yl)-4-(4-(3,4-dimethylphenyl)piperazin-1-yl)butane-1,4-dione;
213 1-(4-(4-chlorophenyl)-6,7-dihydrothieno[3,2-c]pyridin-5(4H)-yl)-4-(4-(3,4-dichlorophenyl)piperazin-1-yl)butane-1,4-dione;
214 1-(4-(4-tert-butylbenzyl)piperazin-1-yl)-4-(4-(4-chlorophenyl)-6,7-dihydro-thieno[3,2-c]pyridin-5(4H)-yl)butane-1,4-dione;
215 1-[4-(3,5-dichloro-4-pyridyl)-1-piperazinyl]-2-methyl-4-(4-phenyl-6,7-dihydro-4H-thieno[3,2-c]pyridin-5-yl)butane-1,4-dione;
216 1-[4-[(4-methoxyphenyl)methyl]-1-piperazinyl]-4-[4-(p-tolyl)-6,7-dihydro-4H-thieno[3,2-c]pyridin-5-yl]butane-1,4-dione;
217 1-[4-[(2-fluorophenyl)methyl]-1-piperazinyl]-4-[4-(p-tolyl)-6,7-dihydro-4H-thieno[3,2-c]pyridin-5-yl]butane-1,4-dione;
218 1-(4-methyl-2-phenyl-1-piperazinyl)-4-[4-(p-tolyl)-6,7-dihydro-4H-thieno[3,2-c]-pyridin-5-yl]butane-1,4-dione;
219 1-(4-phenyl-1-piperidinyl)-4-[4-(p-tolyl)-6,7-dihydro-4H-thieno[3,2-c]pyridin-5-yl]butane-1,4-dione;
220 1-[4-(p-tolyl)-6,7-dihydro-4H-thieno[3,2-c]pyridin-5-yl]-4-[2-(2-pyridyl)-1-pyrrolidinyl]butane-1,4-dione;
221 1-[4-(p-tolyl)-6,7-dihydro-4H-thieno[3,2-c]pyridin-5-yl]-4-[3-(3-pyridyl)-1-pyrrolidinyl]butane-1,4-dione;
222 4-oxo-4-[4-(p-tolyl)-6,7-dihydro-4H-thieno[3,2-c]pyridin-5-yl]-N-[2-(2-pyridyl)-ethyl]butanamide;
223 1-[4-[(4-methoxyphenyl)methyl]-1-piperazinyl]-4-[4-(m-tolyl)-6,7-dihydro-4H-thieno[3,2-c]pyridin-5-yl]butane-1,4-dione;
224 1-[4-[(2-fluorophenyl)methyl]-1-piperazinyl]-4-[4-(m-tolyl)-6,7-dihydro-4H-thieno[3,2-c]pyridin-5-yl]butane-1,4-dione;
225 1-(4-methyl-2-phenyl-1-piperazinyl)-4-[4-(m-tolyl)-6,7-dihydro-4H-thieno[3,2-c]-pyridin-5-yl]butane-1,4-dione;
226 1-[4-(m-tolyl)-6,7-dihydro-4H-thieno[3,2-c]pyridin-5-yl]-4-(4-phenyl-1-piperidinyl)butane-1,4-dione;
227 1-[4-(m-tolyl)-6,7-dihydro-4H-thieno[3,2-c]pyridin-5-yl]-4-[2-(2-pyridyl)-1-pyrrolidinyl]butane-1,4-dione;
228 1-[4-(m-tolyl)-6,7-dihydro-4H-thieno[3,2-c]pyridin-5-yl]-4-[3-(3-pyridyl)-1-pyrrolidinyl]butane-1,4-dione;
229 N-methyl-4-[4-(m-tolyl)-6,7-dihydro-4H-thieno[3,2-c]pyridin-5-yl]-4-oxo-N-[2-(4-pyridyl)ethyl]butanamide;
230 1-[4-(3-fluorophenyl)-6,7-dihydro-4H-thieno[3,2-c]pyridin-5-yl]-4-[4-[(4-methoxyphenyl)methyl]-1-piperazinyl]butane-1,4-dione;
231 1-[4-(3-fluorophenyl)-6,7-dihydro-4H-thieno[3,2-c]pyridin-5-yl]-4-[4-[(2-fluoro-phenyl)methyl]-1-piperazinyl]butane-1,4-dione;
232 4-[4-(m-tolyl)-6,7-dihydro-4H-thieno[3,2-c]pyridin-5-yl]-4-oxo-N-(p-tolyl)-butanamide;
233 N-(2,4-dimethylphenyl)-4-[4-(m-tolyl)-6,7-dihydro-4H-thieno[3,2-c]pyridin-5-yl]-4-oxobutanamide;
234 4-[4-(3-fluorophenyl)-6,7-dihydro-4H-thieno[3,2-c]pyridin-5-yl]-N-(1-methyl-6-indazolyl)-4-oxobutanamide;
235 4-[4-(3-fluorophenyl)-6,7-dihydro-4H-thieno[3,2-c]pyridin-5-yl]-4-oxo-N-(p-tolyl)butanamide;
236 4-[4-(3-fluorophenyl)-6,7-dihydro-4H-thieno[3,2-c]pyridin-5-yl]-4-oxo-N-phenyl-butanamide;
237 N-(2-chlorophenyl)-4-[4-(3-fluorophenyl)-6,7-dihydro-4H-thieno[3,2-c]pyridin-5-yl]-4-oxobutanamide;
238 N-(2,4-dimethylphenyl)-4-[4-(3-fluorophenyl)-6,7-dihydro-4H-thieno[3,2-c]-pyridin-5-yl]-4-oxobutanamide;
239 4-[4-(4-chlorophenyl)-6,7-dihydro-4H-thieno[3,2-c]pyridin-5-yl]-N-(1-methyl-6-indazolyl)-4-oxobutanamide;
240 4-[4-(4-chlorophenyl)-6,7-dihydro-4H-thieno[3,2-c]pyridin-5-yl]-4-oxo-N-(p-tolyl)butanamide;
241 4-[4-(4-chlorophenyl)-6,7-dihydro-4H-thieno[3,2-c]pyridin-5-yl]-N-(4-methoxy-phenyl)-4-oxobutanamide;
242 4-[4-(4-chlorophenyl)-6,7-dihydro-4H-thieno[3,2-c]pyridin-5-yl]-4-oxo-N-phenyl-butanamide;
243 4-[4-(4-chlorophenyl)-6,7-dihydro-4H-thieno[3,2-c]pyridin-5-yl]-N-(2,4-dimethyl-phenyl)-4-oxobutanamide;
244 4-[4-(2,6-dimethylphenyl)-6,7-dihydro-4H-thieno[3,2-c]pyridin-5-yl]-4-oxo-N-[[3-(trifluoromethyl)phenyl]methyl]butanamide;
245 N-(2-cyclohexylethyl)-4-oxo-4-(4-phenyl-6,7-dihydro-4H-thieno[3,2-c]pyridin-5-yl)butanamide;
246 N-(3,3-dimethylbutyl)-4-oxo-4-(4-phenyl-6,7-dihydro-4H-thieno[3,2-c]pyridin-5-yl)butanamide;
247 N-(cyclohexylmethyl)-4-oxo-4-(4-phenyl-6,7-dihydro-4H-thieno[3,2-c]pyridin-5-yl)butanamide;
248 N-[[3-methyl-5-(trifluoromethoxy)phenyl]methyl]-4-oxo-4-(4-phenyl-6,7-dihydro-4H-thieno[3,2-c]pyridin-5-yl)butanamide;
249 N-[[4-methyl-3-(trifluoromethyl)phenyl]methyl]-4-oxo-4-(4-phenyl-6,7-dihydro-4H-thieno[3,2-c]pyridin-5-yl)butanamide;
250 N-[[4-fluoro-3-(trifluoromethyl)phenyl]methyl]-4-oxo-4-(4-phenyl-6,7-dihydro-4H-thieno[3,2-c]pyridin-5-yl)butanamide;
251 4-[4-(2-ethylphenyl)-6,7-dihydro-4H-thieno[3,2-c]pyridin-5-yl]-4-oxo-N-[[3-(trifluoromethyl)phenyl]methyl]butanamide;
252 4-[4-(2-isopropylphenyl)-6,7-dihydro-4H-thieno[3,2-c]pyridin-5-yl]-4-oxo-N-[[3-(trifluoromethyl)phenyl]methyl]butanamide;
253 N-(3-cyclohexylpropyl)-4-oxo-4-(4-phenyl-6,7-dihydro-4H-thieno[3,2-c]pyridin-5-yl)butanamide;
254 4-[4-(3-methyl-2-thienyl)-6,7-dihydro-4H-thieno[3,2-c]pyridin-5-yl]-4-oxo-N-[[3-(trifluoromethyl)phenyl]methyl]butanamide;
255 (1S,2S)-2-[oxo-(4-phenyl-6,7-dihydro-4H-thieno[3,2-c]pyridin-5-yl)methyl]-N-[[3-(trifluoromethyl)phenyl]methyl]-1-cyclopropanecarboxamide;
256 4-[4-(o-tolyl)-6,7-dihydro-4H-thieno[3,2-c]pyridin-5-yl]-4-oxo-N-[[2-(trifluoro-methyl)phenyl]methyl]butanamide;
257 4-[4-(o-tolyl)-6,7-dihydro-4H-thieno[3,2-c]pyridin-5-yl]-4-oxo-N-[[4-(trifluoro-methyl)phenyl]methyl]butanamide;
258 4-(7-butyl-5,7-dihydro-4H-thieno[2,3-c]pyridin-6-yl)-4-oxo-N-[[3-(trifluoro-methyl)phenyl]methyl]butanamide;
259 4-[4-(cyclohexylmethyl)-6,7-dihydro-4H-thieno[3,2-c]pyridin-5-yl]-4-oxo-N-[[3-(trifluoromethyl)phenyl]methyl]butanamide;
260 4-(4-cyclopropyl-6,7-dihydro-4H-thieno[3,2-c]pyridin-5-yl)-4-oxo-N-[[3-(trifluoro-methyl)phenyl]methyl]butanamide;
261 4-oxo-4-(4-phenethyl-6,7-dihydro-4H-thieno[3,2-c]pyridin-5-yl)-N-[[3-(trifluoro-methyl)phenyl]methyl]butanamide;
262 4-[4-(4-fluoro-2-methylphenyl)-6,7-dihydro-4H-thieno[3,2-c]pyridin-5-yl]-4-oxo-N-[[3-(trifluoromethyl)phenyl]methyl]butanamide;
263 4-(4-cyclopentyl-6,7-dihydro-4H-thieno[3,2-c]pyridin-5-yl)-4-oxo-N-[[3-(trifluoro-methyl)phenyl]methyl]butanamide;
264 N-[[2-methyl-3-(trifluoromethyl)phenyl]methyl]-4-oxo-4-(4-phenyl-6,7-dihydro-4H-thieno[3,2-c]pyridin-5-yl)butanamide;
265 4-oxo-4-[4-(3-phenylpropyl)-6,7-dihydro-4H-thieno[3,2-c]pyridin-5-yl]-N-[[3-(trifluoromethyl)phenyl]methyl]butanamide;
266 N-[[2-methyl-5-(trifluoromethyl)phenyl]methyl]-4-oxo-4-(4-phenyl-6,7-dihydro-4H-thieno[3,2-c]pyridin-5-yl)butanamide;
267 N-cyclohexyl-4-oxo-4-(4-phenyl-6,7-dihydro-4H-thieno[3,2-c]pyridin-5-yl)-butanamide;
268 N-(2,2-dimethylpropyl)-4-oxo-4-(4-phenyl-6,7-dihydro-4H-thieno[3,2-c]pyridin-5-yl)butanamide;
269 1-(4-phenyl-6,7-dihydro-4H-thieno[3,2-c]pyridin-5-yl)-4-[7-(trifluoromethyl)-3,4-dihydro-1H-isoquinolin-2-yl]butane-1,4-dione;
270 4-oxo-N-pentyl-4-(4-phenyl-6,7-dihydro-4H-thieno[3,2-c]pyridin-5-yl)-butanamide;
271 1-(4-phenyl-6,7-dihydro-4H-thieno[3,2-c]pyridin-5-yl)-4-[5-(trifluoromethyl)-3,4-dihydro-1H-isoquinolin-2-yl]butane-1,4-dione;
272 4-oxo-4-(4-phenyl-6,7-dihydro-4H-thieno[3,2-c]pyridin-5-yl)-N-[5-(trifluoro-methyl)-1-tetralinyl]butanamide;
273 4-oxo-4-(4-phenyl-6,7-dihydro-4H-thieno[3,2-c]pyridin-5-yl)-N-[7-(trifluoro-methyl)-1-tetralinyl]butanamide;
274 4-[7-(o-tolyl)-5,7-dihydro-4H-thieno[2,3-c]pyridin-6-yl]-4-oxo-N-[[3-(trifluoro-methyl)phenyl]methyl]butanamide;
275 4-(7-cyclohexyl-5,7-dihydro-4H-thieno[2,3-c]pyridin-6-yl)-4-oxo-N-[[3-(trifluoro-methyl)phenyl]methyl]butanamide;
   and physiologically acceptable salts of any of the foregoing.

The substituted tetrahydrothienopyridines according to the invention, and in each case the corresponding acids, bases, salts and solvates, or a compound selected from the group consisting of
1-morpholino-4-(4-(thiophen-2-yl)-6,7-dihydrothieno[3,2-c]pyridin-5(4H)-yl)butane-1,4-dione,
1-(4-acetylpiperazin-1-yl)-4-(4-(thiophen-2-yl)-6,7-dihydrothieno[3,2-c]pyridin-5(4H)-yl)butane-1,4-dione and
1-(3-phenyl-4,5-dihydropyrazol-1-yl)-4-(4-(thiophen-2-yl)-6,7-dihydrothieno-[3,2-c]pyridin-5(4H)-yl)butane-1,4-dione;
   are suitable as pharmaceutical active ingredients in medicaments.

The present invention therefore also provides pharmaceutical compositions comprising a substituted tetrahydrothienopyridine according to the invention of formula (1) wherein R¹ to R¹² have the meanings given above, including compounds selected from the group consisting of
1-morpholino-4-(4-(thiophen-2-yl)-6,7-dihydrothieno[3,2-c]pyridin-5(4H)-yl)-butane-1,4-dione;
1-(4-acetylpiperazin-1-yl)-4-(4-(thiophen-2-yl)-6,7-dihydrothieno[3,2-c]pyridin-5(4H)-yl)butane-1,4-dione, and
1-(3-phenyl-4,5-dihydropyrazol-1-yl)-4-(4-(thiophen-2-yl)-6,7-dihydrothieno-[3,2-c]pyridin-5(4H)-yl)butane-1,4-dione;
   and optionally one or more pharmaceutically acceptable auxiliary substances.

Particular preference is given to pharmaceutical compositions comprising a compound selected from the group consisting of:
2 4-oxo-4-(4-phenyl-6,7-dihydrothieno[3,2-c]pyridin-5(4H)-yl)-N-(3-(trifluoro-methyl)benzyl)butanamide;
3 4-(4-(3-fluorophenyl)-6,7-dihydrothieno[3,2-c]pyridin-5(4H)-yl)-4-oxo-N-(3-(trifluoromethyl)benzyl)butanamide;
4 4-oxo-4-(4-m-tolyl-6,7-dihydrothieno[3,2-c]pyridin-5(4H)-yl)-N-(3-(trifluoro-methyl)benzyl)butanamide;
5 4-oxo-4-(4-p-tolyl-6,7-dihydrothieno[3,2-c]pyridin-5(4H)-yl)-N-(3-(trifluoro-methyl)benzyl)butanamide;
6 4-(4-(2-fluorophenyl)-6,7-dihydrothieno[3,2-c]pyridin-5(4H)-yl)-4-oxo-N-(3-(trifluoromethyl)benzyl)butanamide;
7 4-(4-(4-fluorophenyl)-6,7-dihydrothieno[3,2-c]pyridin-5(4H)-yl)-4-oxo-N-(3-(trifluoromethyl)benzyl)butanamide;
8 4-oxo-4-(4-o-tolyl-6,7-dihydrothieno[3,2-c]pyridin-5(4H)-yl)-N-(3-(trifluoro-methyl)benzyl)butanamide;
9 4-oxo-4-(7-phenyl-4,5-dihydrothieno[2,3-c]pyridin-6(7H)-yl)-N-(3-(trifluoro-methyl)benzyl)butanamide;
10 4-(2-methyl-4-phenyl-6,7-dihydrothieno[3,2-c]pyridin-5(4H)-yl)-4-oxo-N-(3-(trifluoromethyl)benzyl)butanamide;
13 4-oxo-4-(4-phenyl-6,7-dihydrothieno[3,2-c]pyridin-5(4H)-yl)-N-(4-(trifluoro-methyl)benzyl)butanamide;
17 4-oxo-4-(4-phenyl-6,7-dihydrothieno[3,2-c]pyridin-5(4H)-yl)-N-(2-(trifluoro-methyl)benzyl)butanamide;
18 N-(3-fluorobenzyl)-4-oxo-4-(4-phenyl-6,7-dihydrothieno[3,2-c]pyridin-5(4H)-yl)butanamide;
20 4-(3-methyl-4-phenyl-6,7-dihydrothieno[3,2-c]pyridin-5(4H)-yl)-4-oxo-N-(3-(trifluoromethyl)benzyl)butanamide;
21 4-(4-cyclohexyl-6,7-dihydrothieno[3,2-c]pyridin-5(4H)-yl)-4-oxo-N-(3-(trifluoro-methyl)benzyl)butanamide;
22 4-(4-isopropyl-6,7-dihydrothieno[3,2-c]pyridin-5(4H)-yl)-4-oxo-N-(3-(trifluoro-methyl)benzyl)butanamide;
23 4-(4-butyl-6,7-dihydrothieno[3,2-c]pyridin-5(4H)-yl)-4-oxo-N-(3-(trifluoro-methyl)benzyl)butanamide;
27 4-oxo-2-phenyl-4-(4-phenyl-6,7-dihydrothieno[3,2-c]pyridin-5(4H)-yl)-N-(3-(trifluoromethyl)benzyl)butanamide;
28 (R)-2-methyl-4-oxo-4-(4-phenyl-6,7-dihydrothieno[3,2-c]pyridin-5(4H)-yl)-N-(3-(trifluoromethyl)benzyl)butanamide;
29 4-oxo-3-phenyl-4-(4-phenyl-6,7-dihydrothieno[3,2-c]pyridin-5(4H)-yl)-N-(3-(trifluoromethyl)benzyl)butanamide;
30 4-(6,7-dihydrothieno[3,2-c]pyridin-5(4H)-yl)-4-oxo-3-phenyl-N-(3-(trifluoro-methyl)benzyl)butanamide;
31 rac. (1S,2R)-2-(4-phenyl-4,5,6,7-tetrahydrothieno[3,2-c]pyridine-5-carbonyl)-N-(3-(trifluoromethyl)benzyl)cyclohexanecarboxamide;
32 rac. (1S)-2-(4-phenyl-4,5,6,7-tetrahydrothieno[3,2-c]pyridine-5-carbonyl)-N-(3-(trifluoromethyl)benzyl)cyclohexanecarboxamide;
34 (R)-3-methyl-4-oxo-4-(4-phenyl-6,7-dihydrothieno[3,2-c]pyridin-5(4H)-yl)-N-(3-(trifluoromethyl)benzyl)butanamide;
35 (−)-4-oxo-4-(4-phenyl-6,7-dihydrothieno[3,2-c]pyridin-5(4H)-yl)-N-(3-(trifluoro-methyl)benzyl)butanamide;
36 (+)-4-oxo-4-(4-phenyl-6,7-dihydrothieno[3,2-c]pyridin-5(4H)-yl)-N-(3-(trifluoro-methyl)benzyl)butanamide;
37 4-(6,7-dihydrothieno[3,2-c]pyridin-5(4H)-yl)-4-oxo-2-phenyl-N-(3-(trifluoro-methyl)benzyl)butanamide;
40 N-(2-fluoro-3-(trifluoromethyl)benzyl)-4-oxo-4-(4-phenyl-6,7-dihydrothieno-[3,2-c]pyridin-5(4H)-yl)butanamide;
41 N-(3-fluoro-5-(trifluoromethyl)benzyl)-4-oxo-4-(4-phenyl-6,7-dihydrothieno-[3,2-c]pyridin-5(4H)-yl)butanamide;
45 4-(1-methyl-4-phenyl-6,7-dihydrothieno[3,4-c]pyridin-5(4H)-yl)-4-oxo-N-(3-(trifluoromethyl)benzyl)butanamide;
245 N-(2-cyclohexylethyl)-4-oxo-4-(4-phenyl-6,7-dihydro-4H-thieno[3,2-c]pyridin-5-yl)butanamide;
247 N-(cyclohexylmethyl)-4-oxo-4-(4-phenyl-6,7-dihydro-4H-thieno[3,2-c]pyridin-5-yl)butanamide;
249 N-[[4-methyl-3-(trifluoromethyl)phenyl]methyl]-4-oxo-4-(4-phenyl-6,7-dihydro-4H-thieno[3,2-c]pyridin-5-yl)butanamide;
250 N-[[4-fluoro-3-(trifluoromethyl)phenyl]methyl]-4-oxo-4-(4-phenyl-6,7-dihydro-4H-thieno[3,2-c]pyridin-5-yl)butanamide;
251 4-[4-(2-ethylphenyl)-6,7-dihydro-4H-thieno[3,2-c]pyridin-5-yl]-4-oxo-N-[[3-(trifluoromethyl)phenyl]methyl]butanamide;
252 4-[4-(2-isopropylphenyl)-6,7-dihydro-4H-thieno[3,2-c]pyridin-5-yl]-4-oxo-N-[[3-(trifluoromethyl)phenyl]methyl]butanamide;
254 4-[4-(3-methyl-2-thienyl)-6,7-dihydro-4H-thieno[3,2-c]pyridin-5-yl]-4-oxo-N-[[3-(trifluoromethyl)phenyl]methyl]butanamide;
258 4-(7-butyl-5,7-dihydro-4H-thieno[2,3-c]pyridin-6-yl)-4-oxo-N-[[3-(trifluoro-methyl)phenyl]methyl]butanamide;
260 4-(4-cyclopropyl-6,7-dihydro-4H-thieno[3,2-c]pyridin-5-yl)-4-oxo-N-[[3-(trifluoro-methyl)phenyl]methyl]butanamide;
261 4-oxo-4-(4-phenethyl-6,7-dihydro-4H-thieno[3,2-c]pyridin-5-yl)-N-[[3-(trifluoro-methyl)phenyl]methyl]butanamide;
262 4-[4-(4-fluoro-2-methylphenyl)-6,7-dihydro-4H-thieno[3,2-c]pyridin-5-yl]-4-oxo-N-[[3-(trifluoromethyl)phenyl]methyl]butanamide;
263 4-(4-cyclopentyl-6,7-dihydro-4H-thieno[3,2-c]pyridin-5-yl)-4-oxo-N-[[3-(trifluoro-methyl)phenyl]methyl]butanamide;
264 N-[[2-methyl-3-(trifluoromethyl)phenyl]methyl]-4-oxo-4-(4-phenyl-6,7-dihydro-4H-thieno[3,2-c]pyridin-5-yl)butanamide;
265 4-oxo-4-[4-(3-phenylpropyl)-6,7-dihydro-4H-thieno[3,2-c]pyridin-5-yl]-N-[[3-(trifluoromethyl)phenyl]methyl]butanamide;
269 1-(4-phenyl-6,7-dihydro-4H-thieno[3,2-c]pyridin-5-yl)-4-[7-(trifluoromethyl)-3,4-dihydro-1H-isoquinolin-2-yl]butane-1,4-dione;
271 1-(4-phenyl-6,7-dihydro-4H-thieno[3,2-c]pyridin-5-yl)-4-[5-(trifluoromethyl)-3,4-dihydro-1H-isoquinolin-2-yl]butane-1,4-dione;
274 4-[7-(o-tolyl)-5,7-dihydro-4H-thieno[2,3-c]pyridin-6-yl]-4-oxo-N-[[3-(trifluoro-methyl)phenyl]methyl]butanamide;
275 4-(7-cyclohexyl-5,7-dihydro-4H-thieno[2,3-c]pyridin-6-yl)-4-oxo-N-[[3-(trifluoro-methyl)phenyl]methyl]butanamide;
   and physiologically acceptable salts of any of the foregoing.

In addition to at least one compound according to the invention, the pharmaceutical compositions according to the invention optionally comprise suitable additives and/or auxiliary substances, that is to say also carriers, fillers, solvents, diluents, colorings and/or binders, and can be administered as liquid medicament forms in the form of injection solutions, drops or juices, as semi-solid medicament forms in the form of granules, tablets, pellets, patches, capsules, plasters/spray-on plasters or aerosols. The choice of auxiliary substances etc. and the amounts thereof to be used are dependent on whether the medicament is to be administered orally, perorally, parenterally, intravenously, intraperitoneally, intradermally, intramuscularly, intranasally, buccally, rectally or locally, for example to the skin, the mucosa or into the eyes. Preparations in the form of tablets, dragées, capsules, granules, drops, juices and syrups are suitable for oral administration, and solutions, suspensions, readily reconstitutable dry preparations and sprays are suitable for parenteral, topical and inhalatory administration. Compounds according to the invention in a depot, in dissolved form or in a plaster, optionally with the addition of agents that promote penetration through the skin, are suitable percutaneous forms of administration. Forms of preparation for administration orally or percutaneously can release the compounds according to the invention in a delayed manner. The compounds according to the invention can also be administered in parenteral long-term depot forms such as, for example, implants or implanted pumps. In principle, other further active ingredients known to persons skilled in the art can be added to the medicaments according to the invention.

The compounds and compositions according to the invention are suitable for influencing KCNQ2/3 channels and exert an agonistic or antagonistic action, in particular an agonistic action. The compounds and compositions according to the invention are preferably suitable for the treatment of disorders or diseases that are mediated at least in part by KCNQ2/3 channels. The compounds and compositions according to the invention thus are suitable for the treatment or inhibition of disease states or disorders selected from the group consisting of pain, especially pain selected from the group consisting of acute pain, chronic pain, neuropathic pain, muscular pain and inflammatory pain; epilepsy, urinary incontinence, anxiety, dependency, mania, bipolar disorders, migraine, cognitive diseases, dystonia-associated dyskinesias and/or urinary incontinence. The compounds and compositions according to the invention are suitable particularly preferably for the treatment of pain, most particularly preferably of chronic pain, neuropathic pain, inflammatory pain and muscular pain. The compounds and compositions according to the invention are also preferably suitable for the treatment of epilepsy.

The invention further provides the use of at least one substituted tetrahydrothienopyridine according to the invention, including compounds selected from the group consisting of
1-morpholino-4-(4-(thiophen-2-yl)-6,7-dihydrothieno[3,2-c]pyridin-5(4H)-yl)-butane-1,4-dione,
1-(4-acetylpiperazin-1-yl)-4-(4-(thiophen-2-yl)-6,7-dihydrothieno[3,2-c]pyridin-5(4H)-yl)butane-1,4-dione and
1-(3-phenyl-4,5-dihydropyrazol-1-yl)-4-(4-(thiophen-2-yl)-6,7-dihydrothieno-[3,2-c]pyridin-5(4H)-yl)butane-1,4-dione;
   and optionally one or more pharmaceutically acceptable auxiliary substances, in the preparation of a medicament for the treatment of disorders or diseases that are mediated at least in part by KCNQ2/3 channels.

Preference is given to the use of at least one substituted tetrahydrothienopyridine compound according to the invention, or a composition comprising a substituted tetrahydrothienopyridine compound and one or more pharmaceutically acceptable auxiliary substances, for the treatment of pain, especially pain selected from the group consisting of acute pain, chronic pain, neuropathic pain, muscular pain and inflammatory pain; epilepsy, urinary incontinence, anxiety, dependency, mania, bipolar disorders, migraine, cognitive diseases, dystonia-associated dyskinesias and/or urinary incontinence. Particular preference is given to the use of at least one substituted tetrahydrothienopyridine according to the invention, or of a composition comprising a substituted tetrahydrothienopyridine compound and one or more pharmaceutically acceptable auxiliary substances, for the treatment of pain, most particularly preferably chronic pain, neuropathic pain, inflammatory pain and muscular pain. Particular preference is given also to the use of at least one substituted tetrahydrothienopyridine according to the invention, or a composition comprising a substituted tetrahydrothienopyridine compound and one or more pharmaceutically acceptable auxiliary substances, for the treatment of epilepsy.

The effectiveness of the compounds and compositions of the invention against pain can be shown, for example, in the Bennett or Chung model (Bennett, G. J. and Xie, Y. K., A peripheral mononeuropathy in rat that produces disorders of pain sensation like those seen in man, Pain 1988, 33(1), 87-107; Kim, S. H. and Chung, J. M., An experimental model for peripheral neuropathy produced by segmental spinal nerve ligation in the rat, Pain 1992, 50(3), 355-363). The effectiveness against epilepsy can be demonstrated, for example, in the DBA/2 mouse model (De Sarro et al., Naunyn-Schmiedeberg's Arch. Pharmacol. 2001, 363, 330-336).

The substituted tetrahydrothienopyridines according to the invention preferably have a EC₅₀ value of not more than 10 μM or not more than 5 μM, more preferably not more than 3 μM or not more than 2 μM, yet more preferably not more than 1.5 μM or not more than 1 μM, most preferably not more than 0.8 μM or not more than 0.6 μM and especially not more than 0.4 μM or not more than 0.2 μM. Methods for determining the EC₅₀ value are known to persons skilled in the art. The EC₅₀ value is preferably determined by fluorimetry, particularly preferably as described under “Pharmacological Experiments”.

The invention further provides a process for the preparation of the substituted tetrahydrothienopyridines according to the invention.

The chemicals and reaction components used in the reactions described hereinbelow are available commercially or in each case can be prepared by conventional methods known to persons skilled in the art.

### General Preparation Process

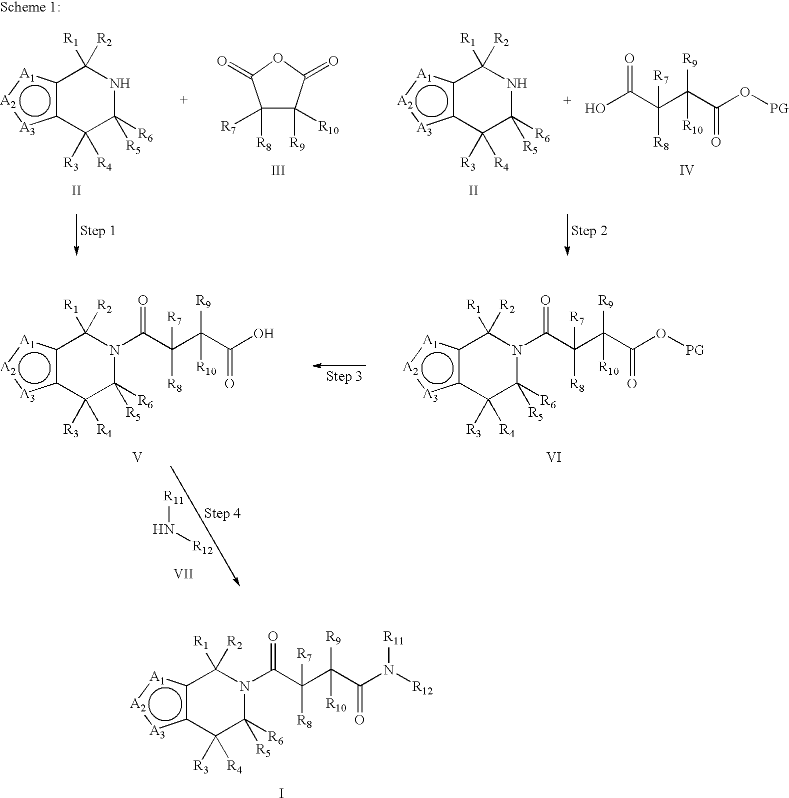

In step 1, amines of formula II are reacted with succinic anhydrides of formula III, in a reaction medium, preferably selected from the group consisting of acetone, acetonitrile, chloroform, dioxane, dichloromethane, ethanol, ethyl acetate, nitrobenzene, methanol and tetrahydrofuran, optionally in the presence of an inorganic base, preferably potassium carbonate, or of an organic base, preferably selected from the group consisting of triethylamine, pyridine, dimethylaminopyridine and diisopropylethylamine, preferably at temperatures of from −20° C. to 160° C., to give carboxylic acids of formula V.

In step 2, carboxylic acids of formula IV wherein PG represents a C₁₋₆-alkyl group, preferably methyl, ethyl, isopropyl or tert-butyl, are reacted with amines of formula II by the processes described under step 4 to give compounds of formula VI.

In step 3, carboxylic acid esters of formula VI wherein PG represents a C₁₋₆-alkyl group, preferably methyl, ethyl, isopropyl or tert-butyl, are cleaved, optionally in a reaction medium, preferably selected from the group consisting of acetone, acetonitrile, chloroform, dioxane, dichloromethane, ethanol, methanol, tetrahydrofuran and water, or in a mixture of these reaction media, optionally in the presence of an inorganic base, preferably LiOH or NaOH, or optionally in the presence of an acid, preferably formic acid, hydrochloric acid or trifluoroacetic acid, optionally in the presence of triethylsilane, triisopropylsilane or ethanediol, preferably at temperatures of from −20° C. to 80° C., to give carboxylic acids of formula V.

In step 4, carboxylic acids of formula V are reacted with amines of formula VII, in a reaction medium, preferably selected from the group consisting of diethyl ether, tetrahydrofuran, acetonitrile, methanol, ethanol, dimethylformamide and dichloromethane, optionally in the presence of at least one coupling reagent, preferably selected from the group consisting of 1-benzotriazolyloxy-tris-(dimethylamino)-phosphonium hexafluorophosphate (BOP), dicyclohexylcarbodiimide (DCC), diisopropylcarbodiimide (DIC), N′-(3-dimethylaminopropyl)-N-ethylcarbo-diimide (EDCI), N-[(dimethylamino)-1H-1,2,3-triazolo[4,5-b]pyridin-1-ylmethylene]-N-methylmethanaminium hexafluorophosphate N-oxide (HATU), O-(benzotriazol-1-yl)-N,N,N′,N′-tetramethyluronium hexafluorophosphate (HBTU) and 1-hydroxy-7-aza-benzotriazole (HOAt), optionally in the presence of at least one inorganic base, preferably selected from the group consisting of potassium carbonate and caesium carbonate, or of an organic base, preferably selected from the group consisting of triethylamine, pyridine, dimethylaminopyridine and diisopropylethylamine, preferably at temperatures of from −70° C. to 150° C., to give compounds of formula I.

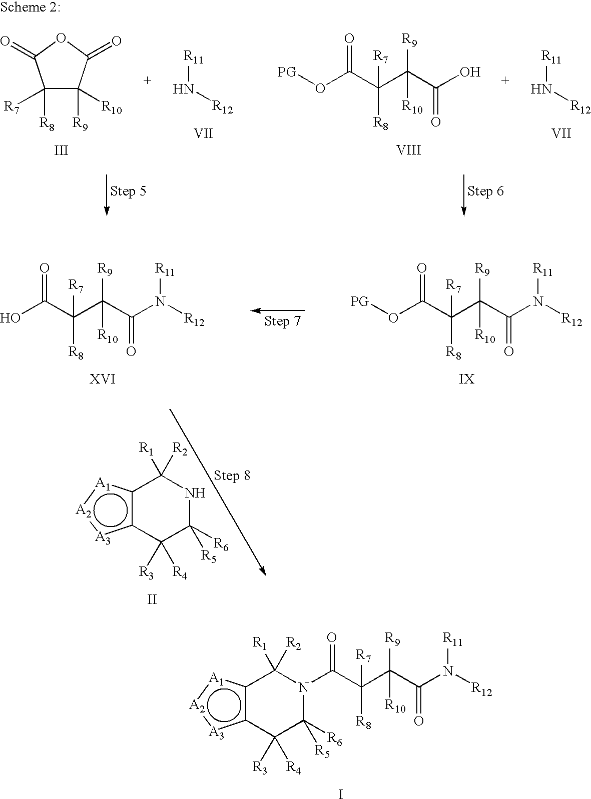

In step 5, amines of formula VII are reacted with succinic anhydrides of formula III according to the processes described under step 1 to give carboxylic acids of formula XVI.

In step 6, amines of formula VII are reacted with carboxylic acids of formula VIII according to the processes described under step 4 to give compounds of formula IX.

In step 7, carboxylic acid esters of formula IX wherein PG represents a C₁₋₆-alkyl group, preferably methyl, ethyl, isopropyl or tert-butyl, are cleaved according to the processes described under step 3 to give carboxylic acids of formula XVI.

In step 8, amines of formula II are reacted with carboxylic acids of formula XVI according to the processes described under step 4 to give compounds of formula I.

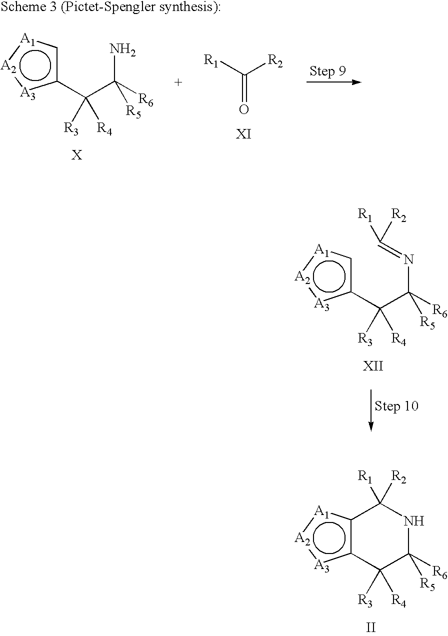

In step 9, amines of formula X are reacted with ketones or aldehydes (R²═H) of formula XI, in a reaction medium, preferably selected from the group consisting of acetonitrile, chloroform, dichloromethane, diethyl ether, ethanol, methanol, tetrahydrofuran, toluene and xylene, optionally in the presence of an inorganic base, preferably potassium carbonate, or an organic base, preferably selected from the group consisting of triethylamine, pyridine, dimethylaminopyridine and diisopropylethylamine, preferably at temperatures of from 0° C. to 160° C., to give imines of formula XII.

In step 10, imines of formula XII are cyclized, optionally in a reaction medium, preferably selected from the group consisting of benzene, ethanol, methanol, toluene, water and xylene, with the addition of an acid, preferably selected from the group consisting of hydrochloric acid, trifluoroacetic acid and trifluoromethanesulfonic acid, preferably at temperatures of from 0° C. to 160° C., to give compounds of formula II.

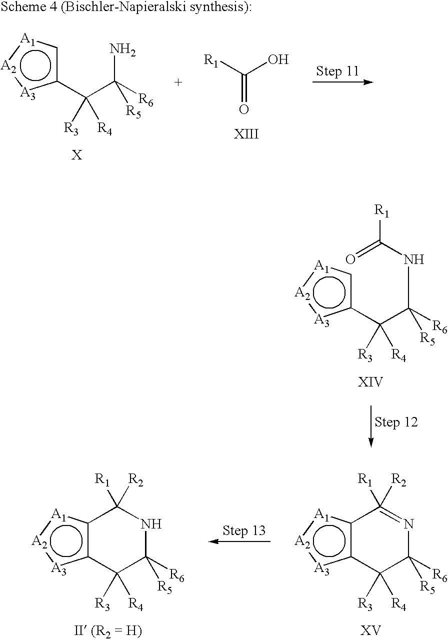

In step 11, amines of formula X are reacted with carboxylic acids of formula XIII according to the processes described under step 4 to give amides of formula XIV.

In step 12, amides of formula XIV are cyclized in a reaction medium, preferably selected from the group consisting of benzene, chloroform, toluene or xylene, in the presence of a suitable cyclizing reagent, preferably phosphoryl trichloride or phosphorus pentachloride, optionally with the addition of phosphorus pentoxide, preferably at temperatures of from 20° C. to 150° C., to give compounds of formula XV.

In step 13, compounds of formula XIV are reduced in a reaction medium, preferably selected from the group consisting of diethyl ether, ethanol, acetic acid, methanol and tetrahydrofuran, in the presence of a suitable reducing agent, preferably selected from the group consisting of sodium borohydride, sodium cyano-borohydride, lithium aluminium hydride and hydrogen, optionally with the addition of a catalyst, preferably selected from the group consisting of palladium, platinum, platinum oxide and Raney nickel, optionally with the addition of an organic base selected from the group consisting of ammonia, triethylamine and diisopropylethyl-amine, preferably at temperatures of from −20° C. to 100° C., to give compounds of formula II′ (R²═H).

### DESCRIPTION OF THE ILLUSTRATIVE SYNTHESES

### Abbreviations

Ac acetyl
AcOH acetic acid
aq. aqueous
brine sat. aq. NaCl sol.
CDI 1,1′-carbonyldiimidazole
d days
DCM dichloromethane
DMAP 4-(dimethylamino)-pyridine
DIPEA diisopropyl-ethyl-amine
EDC N-(3-dimethylaminopropyl)-N′-ethyl-carbodiimide hydrochloride
EA ethyl acetate
ether diethyl ether
equiv. substance equivalent
sat. saturated
h hour(s)
HATU O-(7-aza-benzotriazol-1-yl)-N,N,N′,N′-tetramethyluronium hexafluorophosphate
HOAt 7-aza-1-hydroxy-1H-benzotriazole
HOBt 1-hydroxy-1H-benzotriazole
sol. solution
m/z mass to charge ratio
M molar
MeCN acetonitrile
MeOH methanol
min minutes
MS mass spectrometry
N/A not available
NEt₃ triethylamine
RG retigabine
RT room temperature 23±7° C.
CC column chromatography on silica gel
TFA trifluoroacetic acid
THF tetrahydrofuran
vv volume ratio

### Synthesis of Intermediates

### Synthesis of Intermediate TAM01: 2-(2-Methylthiophen-3-yl)ethylamine

### a) Synthesis of 2-methyl-3-(2-nitrovinyl)thiophene

0.45 equiv. of NH₄OAc was added to a solution of 3.78 g (30.0 mmol) of 2-methyl-thiophene-3-carbaldehyde in nitromethane (45 ml), and stirring was carried out for 2 h at 100° C. After cooling to RT, the mixture was diluted with EA and washed with water and brine. The organic phase was dried over MgSO₄, filtered and concentrated in vacuo. After CC (hexane/EA 20:1) of the residue, 4.16 g (24.6 mmol, 82%) of 2-methyl-3-(2-nitrovinyl)thiophene were obtained.

### b) Synthesis of 2-(2-methylthiophen-3-yl)ethylamine (TAM01)

A solution of 5.08 g (30.0 mmol) of 2-methyl-3-(2-nitrovinyl)thiophene in ether (150 ml) was added dropwise at RT to a solution of 2.1 equiv. of LiAlH₄ in ether (190 ml). When the addition was complete, quenching with a sat. aq. Na₂SO₄ sol. was carried out. The mixture was then filtered over kieselguhr and the filtrate was concentrated in vacuo. After CC (DCM/MeOH 20:1+0.1% NEt₃) of the residue, 2.33 g (16.5 mmol, 55%) of intermediate TAM01 were obtained.

### Synthesis of Intermediate TAM03: 1-Phenyl-2-(thiophen-3-yl)ethylamine hydrochloride

### a) Synthesis of N-methoxy-N-methyl-2-(thiophen-3-yl)acetamide

20.2 g (105.5 mmol) of EDC, 9.50 g (70.3 mmol) of HOBt and 47.8 ml (281.2 mmol) of DIPEA were added at 0° C. to a solution of 10.0 g (70.3 mmol) of 2-(thiophen-3-yl)-acetic acid in DCM (200 ml). After stirring for 20 min at 0° C., 3.08 g (84.4 mmol) of N,O-dimethylhydroxylamine hydrochloride were added. Stirring was then carried out for a further 16 h at RT. The mixture was then quenched with a sat. aq. NH₄Cl sol. and extracted with DCM. The organic phase was washed with brine, dried over Na₂SO₄, filtered and concentrated in vacuo. After CC (hexane/EA 17:3), 9.98 g (53.9 mmol, 77%) of N-methoxy-N-methyl-2-(thiophen-3-yl)acetamide were obtained.

### b) Synthesis of 1-phenyl-2-(thiophen-3-yl)ethanone

53.4 ml (53.4 mmol, 1M in THF) of phenylmagnesium bromide solution were added dropwise at 0° C. to a solution of 9.9 g (53.4 mmol) of N-methoxy-N-methyl-2-(thiophen-3-yl)acetamide in ether (100 ml). Stirring was then carried out for 3 h at RT, followed by quenching, while cooling with ice, with a sat. aq. NH₄Cl sol. The mixture was extracted with EA and the organic phase was washed with brine, dried over Na₂SO₄, filtered and concentrated in vacuo. The resulting crude product (8.98 g, 44.4 mmol, 83%) 1-phenyl-2-(thiophen-3-yl)ethanone was reacted further without additional purification.

### c) Synthesis of 1-phenyl-2-(thiophen-3-yl)ethanone-oxime

14.56 g (177.6 mmol) of sodium acetate and 6.17 g (88.8 mmol) of hydroxylamine hydrochloride were added to a solution of 8.98 g (44.4 mmol) of 1-phenyl-2-(thiophen-3-yl)ethanone in EtOH (220 ml), and the mixture was heated for 4 h at 85° C. Filtration through Celite was then carried out, and the filtrate was concentrated in vacuo. The residue was taken up in EA, dried over Na₂SO₄, filtered and concentrated in vacuo. There were obtained as residue 7.52 g (34.6 mmol, 78%) of 1-phenyl-2-(thiophen-3-yl)ethanone-oxime, which was reacted further without additional purification.

### d) Synthesis of 1-phenyl-2-(thiophen-3-yl)ethylamine hydrochloride (TAM03)

3.81 g of Raney nickel were added to a solution of 7.52 g (34.6 mmol) of 1-phenyl-2-(thiophen-3-yl)ethanone-oxime dissolved in EtOH (760 ml). The reaction solution was then stirred for 3 d under a hydrogen atmosphere at 4.14 bar and 30° C. Filtration over Celite was then carried out, and the filtrate was concentrated in vacuo. The resulting residue was dissolved in 1M aq. hydrochloric acid and washed with EA. The aqueous phase was adjusted to pH 8-10 with a sat. aq. Na₂CO₂ sol., while cooling with ice, and extraction with EA was then carried out. The organic phase was dried over Na₂SO₄, filtered and concentrated in vacuo. The resulting residue was dissolved in dioxane (40 ml), and a sat. HCl sol. in dioxane (115 ml) was added at 0° C. (ice bath). After 2 h stirring at RT, concentration in vacuo was carried out. 3.1 g (12.8 mmol, 37%) of intermediate TAMO3 were obtained.

### Synthesis of Intermediate SAMN04: 4-(2-Fluoro-phenyl)-4,5,6,7-tetrahydro-thieno[3,2-c]pyridine

4.8 ml (34.6 mmol) of NEt₃ and 8.2 ml (78.6 mmol) of 2-fluoro-benzaldehyde were added to a solution of 9.2 ml (78.6 mmol) of 2-thiophen-2-yl-ethylamine in EtOH (80 ml), and stirring was carried out for 72 h at 50° C. The mixture was then concentrated in vacuo. The residue was taken up in TFA (300 ml), and stirring was carried out for a further 72 h at RT. After concentration in vacuo, the residue was taken up in DCM and washed with a 2M aq. NaOH sol. The organic phase was dried over MgSO₄, filtered and concentrated in vacuo. After CC (EA/hexane 3:1) of the residue, 12.2 g (52.3 mmol, 66%) of intermediate SAMN04 were obtained.

### Synthesis of Intermediate SAMN08: 2-methyl-4-phenyl-4,5,6,7-tetrahydrothieno-[3,2-c]pyridine

4 Å molecular sieve was added to a solution of 2.0 g (14.1 mmol) of 2-(5-methylthien-2-yl)-ethylamine and 1.42 ml (14.1 mmol) of benzaldehyde in toluene (75 ml), and the mixture was heated under reflux for 16 h with a water separator. The molecular sieve was then filtered off and washed with toluene. The filtrate was concentrated in vacuo. The residue was taken up in TFA (54 ml) and stirring was carried out for 6 d at RT. The mixture was then concentrated in vacuo. The resulting residue was taken up in EA and washed with a 2M aq. NaOH sol. The organic phase was dried over MgSO₄, filtered and concentrated in vacuo. After CC (EA/hexane 4:1) of the residue, 1.98 g (8.6 mmol, 61%) of intermediate SAMN08 were obtained.

### Synthesis of Intermediate SAMN10: 5-phenyl-4,5,6,7-tetrahydrothieno[2,3-c]-pyridine

A solution of 3.1 g (12.8 mmol) of intermediate TAM03 in a mixture of formaldehyde/-AcOH (1:1 vv, 230 ml) was heated for 1 h under reflux. The mixture was then diluted with water and neutralized with liquid ammonia. Extraction with EA was then carried out, and the organic phase was washed with brine, dried over Na₂SO₄, filtered and concentrated in vacuo. After CC (hexane/EA 7:3) of the residue, 1.27 g (5.9 mmol, 46%) of intermediate SAMN10 were obtained.

### Synthesis of Intermediate SAMN13: 4-(2-Isopropylphenyl)-4,5,6,7-tetrahydro-thieno[3,2-c]pyridine

4 Å molecular sieve was added to a solution of 2.5 g (19.7 mmol) of 2-thiophen-2-yl-ethylamine and 2.9 g (19.7 mmol) of 2-isopropyl-benzaldehyde in toluene (40 ml), and the mixture was heated for 16 h under reflux with a water separator. The molecular sieve was then filtered off and washed with toluene. The filtrate was concentrated in vacuo. The residue was taken up in TFA (40 ml) and stirred for 72 h at 90° C. The mixture was then concentrated in vacuo. A 2M aq. NaOH sol. was added to the resulting residue, and extraction with DCM was then carried out. The organic phase was dried over MgSO₄, filtered and concentrated in vacuo. After CC (EA/hexane 1:1) of the residue, 3.28 g (12.7 mmol, 64%) of intermediate SAMN13 were obtained.

### Synthesis of Intermediate ASP01: 4-Methoxy-2-methyl-4-oxo-butyric acid

A stirred solution of 4-methoxy-2-methylene-4-oxo-butyric acid (5.0 g, 34.7 mmol) in ethanol (100 ml) was deoxygenated for 15 min with N₂; palladium (10% on carbon, 1.85 g, 1.74 mmol) was then added and deoxygenation with N₂ was carried out again for 15 min. The reaction mixture was stirred for 16 h under a H₂ atmosphere and then filtered over Celite. The solvent was removed using a rotary evaporator. The resulting crude intermediate ASP01 was used further without additional purification.

### Synthesis of the Intermediate ASP02: 4-tert-Butoxy-3-methyl-4-oxobutyric acid

### a) Synthesis of 1-tert-butyl 4-methyl 2-methylsuccinate

A solution of intermediate ASP01 (20.2 g, 138.0 mmol), DMAP (8.44 g, 69.0 mmol) and tert-butanol (14.45 ml, 152.0 mmol) in DCM (500 ml) was cooled to 0° C., and EDC (29.1 g, 152.0 mmol) was added. The reaction mixture was stirred for 16 h at RT. When the reaction was ended, the mixture was washed with brine (500 ml). The organic phase was then dried over Na₂SO₄, filtered and concentrated to dryness. The resulting crude product, 1-tert-butyl 4-methyl 2-methylsuccinate, was used further without additional purification.

### b) Synthesis of 4-tert-butoxy-3-methyl-4-oxobutyric acid (ASP02)

LiOH.H₂O (5.19 g, 124.0 mmol) was added to a solution of (25.0 g, 124.0 mmol) 1-tert-butyl 4-methyl 2-methylsuccinate in a mixture of MeOH (85 ml), THF (85 ml) and water (85 ml), and the reaction mixture was stirred for 16 h at RT. When the reaction was ended, the solvent was removed using a rotary evaporator. The residue was taken up in aq. HCl (1M, 130 ml) and extracted twice with DCM (150 ml). The combined organic phases were dried over Na₂SO₄, filtered and then concentrated to dryness. The resulting crude intermediate ASP02 was used further without additional purification.

### Synthesis of Intermediate SAAS01: 4-Oxo-4-(4-phenyl-6,7-dihydrothieno[3,2-c]-pyridin-5(4H)-yl)butyric acid

A solution of 8.0 g (37.2 mmol) of 4-phenyl-4,5,6,7-tetrahydrothieno[3,2-c]pyridine in MeCN (20 ml) was added to a suspension of 13.0 g (130.1 mmol) of succinic anhydride in MeCN (80 ml). 10.3 ml (74.3 mmol) of NEt₃ were then added to the reaction solution, and stirring was carried out for 72 h at RT. The mixture was then concentrated in vacuo; the residue was taken up in EA, and water was added thereto. Extraction with a sat. aq. NaHCO₃ sol. was then carried out several times. The combined aqueous phases were adjusted to pH 6-8 with 2N hydrochloric acid. Extraction with EA was then carried out. The organic phase was washed several times with water; activated carbon was added, and stirring was carried out for 15 min. The mixture was then filtered over kieselguhr and the filtrate was dried over MgSO₄, filtered and concentrated in vacuo. 7.08 g (22.4 mmol, 60%) of intermediate SAAS01 were obtained as residue.

### Synthesis of Intermediate SAAS02: (3R)-3-methyl-4-oxo-4-(4-phenyl-6,7-dihydrothieno[3,2-c]pyridin-5(4H)-yl)butyric acid

### a) Synthesis of (3R)-3-methyl-4-oxo-4-(4-phenyl-6,7-dihydrothieno[3,2-c]pyridin-5(4H)-yl)butyric acid methyl ester

1.53 g (7.1 mmol) of 4-phenyl-4,5,6,7-tetrahydrothieno[3,2-c]pyridine, 2.46 g (6.5 mmol) of HATU and 1.7 ml (12.3 mmol) of NEt₃ were added in succession to a solution of 945 mg (6.5 mmol) of (R)-4-methoxy-2-methyl-4-oxobutyric acid in THF (50 ml). After 16 h stirring at RT, the reaction solution was diluted with EA (30 ml), and washing was carried out twice with a sat. aq. NH₄Cl sol. and twice with a 1N aq. NaHCO₃ sol. The organic phase was dried over MgSO₄, filtered and concentrated in vacuo. After CC (EA) of the residue, 1.14 g (3.3 mmol, 51%) of (3R)-3-methyl-4-oxo-4-(4-phenyl-6,7-dihydrothieno[3,2-c]pyridin-5(4H)-yl)butyric acid methyl ester were obtained.

### b) Synthesis of (3R)-3-methyl-4-oxo-4-(4-phenyl-6,7-dihydrothieno[3,2-c]pyridin-5(4H)-yl)butyric acid (SAAS02)

A solution of 1.14 g (3.3 mmol) of (3R)-3-methyl-4-oxo-4-(4-phenyl-6,7-dihydrothieno-[3,2-c]pyridin-5(4H)-yl)butyric acid methyl ester in a mixture of THF/MeOH (1:1 vv, 50 ml) was cooled to 0° C. (ice bath), and 25 ml of a 1M aq. LiOH sol. were added. Stirring was then carried out for 10 min, the ice bath was removed, and stirring was carried out for a further 150 min. The mixture was then diluted with EA (100 ml), and 1M aq. hydrochloric acid (25 ml) was added. The phases were separated and the aqueous phase was extracted with EA. The combined organic phases were dried over MgSO₄, filtered and concentrated in vacuo. After CC (EA) of the residue, 795 mg (2.4 mmol, 73%) of intermediate SAAS02 were obtained.

### Synthesis of Intermediate SAAS07: 4-(4-(4-Chlorophenyl)-6,7-dihydrothieno-[3,2-c]pyridin-5(4H)-yl)-4-oxobutyric acid

### a) Synthesis of 4-(4-(4-chlorophenyl)-6,7-dihydrothieno[3,2-c]pyridin-5(4H)-yl)-4-oxobutyric acid methyl ester

8.2 ml (59.0 mmol) of NEt₃ were added to a solution of 9.82 g (39.3 mmol) of intermediate SAMN12 in DCM (250 ml). A solution of 6.50 g (43.2 mmol) of 4-chloro-4-oxobutyric acid methyl ester in DCM (100 ml) was then added dropwise. After 3 h stirring at RT, washing with 0.5 M aq. hydrochloric acid (250 ml) and with a sat. aq. NaHCO₃ sol. (250 ml) was carried out. The organic phase was dried over Na₂SO₄, filtered and concentrated in vacuo. After CC with the residue (heptane/EA 3:1), 13.9 g (38.2 mmol, 97%) of 4-(4-(4-chlorophenyl)-6,7-dihydrothieno[3,2-c]pyridin-5(4H)-yl)-4-oxobutyric acid methyl ester were obtained.

### b) Synthesis of 4-(4-(4-chlorophenyl)-6,7-dihydrothieno[3,2-c]pyridin-5(4H)-yl)-4-oxobutyric acid (SAAS07)

A 6M aq. NaOH sol. (30 ml) was added to a solution of 11.0 g (30.3 mmol) of 4-(4-(4-chlorophenyl)-6,7-dihydrothieno[3,2-c]pyridin-5(4H)-yl)-4-oxo-butyric acid methyl ester in a mixture of THF/MeOH (1:1 vv, 200 ml), and the mixture was then stirred for 2 h at RT. Most of the organic solvents were then removed in vacuo. The mixture was then neutralized with 6M aq. hydrochloric acid. Extraction with DCM (2×200 ml) was then carried out. The combined organic phases were dried over Na₂SO₄, filtered and concentrated in vacuo. The resulting crude intermediate SAAS07 was used further without additional purification.

### Synthesis of Intermediate SAAS09: 2-methyl-4-oxo-4-(4-phenyl-6,7-dihydro-thieno[3,2-c]pyridin-5(4H)-yl)butyric acid

### a) Synthesis of 2-methyl-4-oxo-4-(4-phenyl-6,7-dihydrothieno[3,2-c]pyridin-5(4H)-yl)butyric acid tert-butyl ester

9.3 g (48.5 mmol) of EDC were added to a solution of 6.95 g (32.3 mmol) of 4-phenyl-4,5,6,7-tetrahydrothieno[3,2-c]pyridine, 6.38 g (33.9 mmol) of intermediate ASP02, 436 mg (3.2 mmol) of HOAt and 11 ml (64.6 mmol) of DIPEA in DCM (300 ml), and the mixture was stirred for 16 h at RT. Washing with 0.5 M aq. hydrochloric acid (250 ml) and a sat. aq. NaHCO₃ sol. (250 ml) was then carried out. The organic phase was dried over Na₂SO₄, filtered and concentrated in vacuo. After CC (heptane/EA 4:1) of the residue, 9.67 g (25.1 mmol, 78%) of 2-methyl-4-oxo-4-(4-phenyl-6,7-dihydrothieno[3,2-c]pyridin-5(4H)-yl)butyric acid tert-butyl ester were obtained.

### b) Synthesis of 2-methyl-4-oxo-4-(4-phenyl-6,7-dihydrothieno[3,2-c]pyridin-5(4H)-yl)butyric acid (SAA09)

A 6M aq. NaOH sol. (25 ml) was added to a solution of 9.64 g (25.0 mmol) of 2-methyl-4-oxo-4-(4-phenyl-6,7-dihydrothieno-[3,2-c]pyridin-5(4H)-yl)butyric acid tert-butyl ester in a mixture of THF/MeOH (1:1 vv, 165 ml), and the mixture was then stirred for 2 h at RT. Most of the organic solvents were then removed in vacuo. The mixture was then neutralized with 6M aq. hydrochloric acid. Extraction with DCM (2×200 ml) was then carried out. The combined organic phases were dried over Na₂SO₄, filtered and concentrated in vacuo. 7.82 g (23.7 mmol, 95%) of intermediate SAAS09 were obtained as residue and were used further without additional purification.

### Synthesis of Intermediate PAAS01: 4-Oxo-4-(3-(trifluoromethyl)benzylamino)-butyric acid

A solution of 15.0 g (85.6 mmol) of (3-(trifluoromethyl)phenyl)methylamine in ether (40 ml) was added dropwise in the course of 30 min to a suspension of 7.5 g (85.6 mmol) of succinic anhydride in ether (450 ml). Stirring was then carried out for 72 h at RT. The resulting precipitate was filtered off and dried. After CC (EA/MeOH 1:1) of the residue, 10.4 g (37.8 mmol, 44%) of intermediate PAAS01 were obtained.

### Synthesis of Intermediates PAAS03: 4-Oxo-2-phenyl-4-(3-(trifluoromethyl)-benzylamino)butyric acid and PAAS04: 4-Oxo-3-phenyl-4-(3-(trifluoromethyl)-benzylamino)butyric acid

A solution of 5.0 g (28.4 mmol) of (3-(trifluoromethyl)phenyl)methylamine in ether (50 ml) was added dropwise in the course of 30 min to a suspension of 5.0 g (28.4 mmol) of 3-phenyldihydrofuran-2,5-dione in ether (100 ml). The resulting precipitate was filtered off and dried. After repeated CC (TBME/hexane 4:1, then TBME/EA 1:1, then EA/MeOH 9:1) of the residue, 1.27 g (3.6 mmol, 13%) of intermediate PAAS03 and 372 mg (1.1 mmol, 4%) of intermediate PAAS04 were obtained.

### Synthesis of Intermediate PAAS05: (R)-3-methyl-4-oxo-4-(3-(trifluoromethyl)-benzylamino)-butyric acid

### a) Synthesis of (R)-3-methyl-4-oxo-4-(3-(trifluoromethyl)benzylamino)butyric acid methyl ester

1.32 g (7.5 mmol) of 3-(trifluoromethyl)phenyl)methylamine, 2.60 g (6.8 mmol) of HATU and 1.8 ml (13.0 mmol) of NEt₃were added in succession to a solution of 1.0 g (6.8 mmol) of (R)-4-methoxy-2-methyl-4-oxobutyric acid in THF (50 ml). After 16 h stirring at RT, the reaction solution was diluted with EA (30 ml), and washing was carried out twice with a sat. aq. NH₄Cl sol. and twice with a 1N aq. NaHCO₃ sol. The organic phase was dried over Na₂SO₄, filtered and concentrated in vacuo. After CC (EA) of the residue, 1.30 g (4.3 mmol, 63%) of (R)-3-methyl-4-oxo-4-(3-(trifluoro-methyl)benzylamino)butyric acid methyl ester were obtained.

### b) Synthesis of (R)-3-methyl-4-oxo-4-(3-(trifluoromethyl)benzylamino)butyric acid (PAAS05)

A solution of 1.29 g (4.3 mmol) of (R)-3-methyl-4-oxo-4-(3-(trifluoromethyl)benzyl-amino)butyric acid methyl ester in a mixture of THF/MeOH (1:1 vv, 70 ml) was cooled to 0° C. (ice bath), and 35 ml of a 1M aq. LiOH sol. were added. Stirring was then carried out for 10 min, the ice bath was removed, and stirring was carried out for a further 120 min. The mixture was then diluted with EA (100 ml), and 1M aq. hydrochloric acid (25 ml) was added. The phases were separated and the aqueous phase was extracted with EA. The combined organic phases were dried over Na₂SO₄, filtered and concentrated in vacuo. After CC (EA) of the residue, 185 mg (0.6 mmol, 15%) of intermediate PAAS05 were obtained.

### Synthesis of Further Intermediates

The synthesis of further intermediates was carried out according to the processes already described. Table 1 shows which compound was prepared by which process. It will be clear to persons skilled in the art which starting materials were used in each case.

TABLE 1

| | | Preparation | |
|---|---|---|---|
| | | analogous to | Yield |
| Intermediate | Chemical name | intermediate | [%] |
| TAM02 | 2-(4-Methylthiophen-2-yl)ethylamine | TAM01 | 21 |
| SAMN01 | 4-(2-Methyl-phenyl)-4,5,6,7- | SAMN08 | 35 |
| | tetrahydrothieno[3,2-c]pyridine | | |
| SAMN02 | 4-(3-Methyl-phenyl)-4,5,6,7- | SAMN04 | 28 |
| | tetrahydrothieno[3,2-c]pyridine | | |
| SAMN03 | 4-(4-Methyl-phenyl)-4,5,6,7- | SAMN08 | 28 |
| | tetrahydrothieno[3,2-c]pyridine | | |
| SAMN05 | 4-(3-Fluoro-phenyl)-4,5,6,7- | SAMN08 | 82 |
| | tetrahydrothieno[3,2-c]pyridine | | |
| SAMN06 | 4-(4-Fluoro-phenyl)-4,5,6,7- | SAMN08 | 22 |
| | tetrahydrothieno[3,2-c]pyridine | | |
| SAMN07 | 3-Methyl-4-phenyl-4,5,6,7- | SAMN08 | 26 |
| | tetrahydrothieno[3,2-c]pyridine | | |
| SAMN09 | 7-Phenyl-4,5,6,7-tetrahydrothieno- | SAMN08 | 51 |
| | [2,3-c]pyridine | | |
| SAMN11 | 1-Methyl-4-phenyl-4,5,6,7- | SAMN08 | 18 |
| | tetrahydrothieno[3,4-c]pyridine | | |
| SAMN12 | 4-(4-Chlorophenyl)-4,5,6,7- | SAMN04 | N/A |
| | tetrahydrothieno[3,2-c]pyridine | | |
| SAMN14 | 7-o-Tolyl-4,5,6,7-tetrahydrothieno- | SAMN13 | N/A |
| | [2,3-c]pyridine | | |
| SAMN15 | 7-Cyclohexyl-4,5,6,7- | SAMN13 | N/A |
| | tetrahydrothieno[2,3-c]pyridine | | |
| SAMN16 | 7-Butyl-4,5,6,7-tetrahydrothieno- | SAMN13 | N/A |
| | [2,3-c]pyridine | | |
| SAMN17 | 4-(2-Ethyl-ylphenyl)-4,5,6,7- | SAMN13 | 68 |
| | tetrahydrothieno[3,2-c]pyridine | | |
| SAMN18 | 4-(2,6-Dimethylphenyl)-4,5,6,7- | SAMN13 | 3 |
| | tetrahydrothieno[3,2-c]pyridine | | |
| SAMN19 | 4-(3-Methylthiophen-2-yl)-4,5,6,7- | SAMN13 | 2 |
| | tetrahydrothieno[3,2-c]pyridine | | |
| SAMN20 | 4-Phenethyl-4,5,6,7- | SAMN13 | N/A |
| | tetrahydrothieno[3,2-c]pyridine | | |
| SAMN21 | 4-(3-Phenylpropyl)-4,5,6,7- | SAMN13 | N/A |
| | tetrahydrothieno[3,2-c]pyridine | | |
| SAMN22 | 4-Cyclopropyl-4,5,6,7- | SAMN13 | N/A |
| | tetrahydrothieno[3,2-c]pyridine | | |
| SAMN23 | 4-Cyclopentyl-4,5,6,7- | SAMN13 | N/A |
| | tetrahydrothieno[3,2-c]pyridine | | |
| SAMN24 | 4-(Cyclohexylmethyl)-4,5,6,7- | SAMN13 | N/A |
| | tetrahydrothieno[3,2-c]pyridine | | |
| SAMN25 | 4-(4-Fluoro-2-methylphenyl)-4,5,6,7- | SAMN13 | N/A |
| | tetrahydrothieno[3,2-c]pyridine | | |
| SAAS03 | 4-Oxo-4-(4-m-tolyl-6,7- | SAAS07 | N/A |
| | dihydrothieno[3,2-c]pyridin-5(4H)- | | |
| | yl)butyric acid | | |
| SAAS04 | 4-Oxo-4-(4-p-tolyl-6,7- | SAAS07 | N/A |
| | dihydrothieno[3,2-c]pyridin-5(4H)- | | |
| | yl)butyric acid | | |
| SAAS05 | 4-(4-(3-Fluorophenyl)-6,7- | SAAS07 | N/A |
| | dihydrothieno[3,2-c]pyridin- | | |
| | 5(4H)-yl)-4-oxobutyric acid | | |
| SAAS06 | 4-(4-(4-Fluorophenyl)-6,7- | SAAS07 | N/A |
| | dihydrothieno[3,2-c]pyridin- | | |
| | 5(4H)-yl)-4-oxobutyric acid | | |
| SAAS08 | 3-Methyl-4-oxo-4-(4-phenyl-6,7- | SAAS09 | N/A |
| | dihydrothieno[3,2-c]pyridin-5(4H)- | | |
| | yl)butyric acid | | |
| SAAS10 | 4-Oxo-4-(4-o-tolyl-6,7- | SAAS01 | 45 |
| | dihydrothieno[3,2-c]pyridin-5(4H)- | | |
| | yl)butyric acid | | |
| PAAS02 | 4-(3,4-Difluorobenzylamino)-4- | PASS01 | 39 |
| | oxobutyric acid | | |
| PAAS06 | rac. (1R,2S)-2-(3-(Trifluoromethyl)- | PASS01 | 98 |
| | benzylcarbamoyl)cyclopropane- | | |
| | carboxylic acid | | |
| PAAS07 | rac. (1R,2S)-2-(3-(Trifluoromethyl)- | PASS01 | 88 |
| | benzylcarbamoyl)cyclopentane- | | |
| | carboxylic acid | | |
| PAAS08 | rac. (1R,2S)-2-(3-(Trifluoromethyl)- | PASS01 | 76 |
| | benzylcarbamoyl)cyclohexane- | | |
| | carboxylic acid | | |
| PAAS09 | rac. (1S,2S)-2-(3-Trifluoromethyl)- | PASS01 | 69 |
| | benzylcarbamoyl)cyclohexane- | | |
| | carboxylic acid | | |
| PASS10 | rac. (1S,2S)-2-(3-(Trifluoromethyl)- | PASS05 | 76 |
| | benzylcarbamoyl)cyclopropane- | | |
| | carboxylic acid | | |

### Synthesis of Illustrative Compounds

### Synthesis of Illustrative Compound 2: 4-Oxo-4-(4-phenyl-6,7-dihydrothieno-[3,2-c]pyridin-5(4H)-yl)-N-(3-(trifluoromethyl)benzyl)butanamide

To a solution of 275 mg (1.0 mmol) of intermediate PAAS01 in THF (8 ml) there were added in succession 236 mg (1.1 mmol) of 4-phenyl-4,5,6,7-tetrahydrothieno[3,2-c]-pyridine, 380 mg (1.0 mmol) of HATU and 263 μl (1.9 mmol) of NEt₃. After 48 h stirring at RT, the reaction solution was diluted with EA (30 ml), and washing was carried out twice with 2N aq. hydrochloric acid and twice with a 1N aq. NaHCO₃ sol. The organic phase was dried over MgSO₄, filtered and concentrated in vacuo. After CC (EA) of the residue, 386 mg (0.8 mmol, 82%) of illustrative compound 2 were obtained. MS: m/z 473.1 [M+H]⁺.

### Synthesis of Illustrative Compound 3: 4-(4-(3-Fluorophenyl)-6,7-dihydrothieno-[3,2-c]pyridin-5(4H)-yl)-4-oxo-N-(3-(trifluoromethyl)benzyl)butanamide

To a solution of 349 mg (1.27 mmol) of intermediate PAAS01 in THF (10 ml) there were added in succession 325 mg (1.40 mmol) of intermediate SAMN05, 482 mg (1.27 mmol) of HATU and 334 μl (2.41 mmol) of NEt₃. After 48 h stirring at RT, the reaction solution was diluted with EA (30 ml), and washing was carried out twice with 2N aq. hydrochloric acid and twice with a 1N aq. NaHCO₃ sol. The organic phase was dried over Na₂SO₄, filtered and concentrated in vacuo. 377 mg (0.77 mmol, 61%) of illustrative compound 3 were obtained as residue. MS: m/z 491.1 [M+H]⁺.

### Synthesis of Illustrative Compound 9: 4-Oxo-4-(7-phenyl-4,5-dihydrothieno-[2,3-c]pyridin-6(7H)-yl)-N-(3-(trifluoromethyl)benzyl)butanamide

To a solution of 300 mg (1.09 mmol) of intermediate PAAS01 in THF (8 ml) there were added in succession 258 mg (1.20 mmol) of intermediate SAMN09, 414 mg (1.09 mmol) of HATU and 287 μl (2.07 mmol) of NEt₃. After 72 h stirring at RT, the reaction solution was diluted with EA (30 ml), and washing was carried out twice with 2N aq. hydrochloric acid and twice with a 1N aq. NaHCO₃ sol. The organic phase was dried over Na₂SO₄, filtered and concentrated in vacuo. Recrystallization of the residue from EA yielded 280 mg (0.59 mmol, 54%) of illustrative compound 9. MS: m/z 473.1 [M+H]⁺.

### Synthesis of Illustrative Compound 11: N-(4-Methylbenzyl)-4-oxo-4-(4-phenyl-6,7-dihydrothieno[3,2-c]pyridin-5(4H)-yl)butanamide

To a solution of 400 mg (1.27 mmol) of intermediate SAAS01 in THF (10 ml) there were added in succession 169 mg (1.40 mmol) of (4-methylphenyl)-methylamine, 482 mg (1.27 mmol) of HATU and 334 μl (2.41 mmol) of NEt₃. After 24 h stirring at RT, the reaction solution was diluted with EA (30 ml), and washing was carried out twice with 2N aq. hydrochloric acid and twice with a 1N aq. NaHCO₃ sol. The organic phase was dried over Na₂SO₄, filtered and concentrated in vacuo. Recrystallization of the residue from EA yielded 239 mg (0.57 mmol, 45%) of illustrative compound 11. MS: m/z 419.2 [M+H]⁺.

### Synthesis of Illustrative Compound 12: N-Benzyl-4-oxo-4-(4-phenyl-6,7-dihydro-thieno[3,2-c]pyridin-5(4H)-yl)butanamide

A solution of 400 mg (1.27 mmol) of intermediate SAAS01 and 216 mg (1.33 mmol) of CDI in DCM (10 ml) was stirred for 1 h at RT. A solution of 138 μl (1.27 mmol) of benzylamine in DCM (10 ml) was then added, and stirring was carried out for a further 16 h at RT. The reaction solution was then washed three times with a sat. aq. NH₄Cl sol. and three times with a sat. aq. NaHCO₃ sol. The organic phase was dried over MgSO₄, filtered and concentrated in vacuo. Recrystallization of the residue from EA yielded 244 mg (0.60 mmol, 47%) of illustrative compound 12. MS: m/z 405.2 [M+H]⁺.

### Synthesis of Illustrative Compound 24: N-Methyl-4-oxo-4-(4-phenyl-6,7-dihydro-thieno[3,2-c]pyridin-5(4H)-yl)-N-(3-(trifluoromethyl)benzyl)butanamide

A solution of 400 mg (1.27 mmol) of intermediate SAAS01 and 216 mg (1.33 mmol) of CDI in THF (13 ml) was stirred for 1 h at RT. A solution of 219 μl (1.27 mmol) of N-methyl-1-(3-(trifluoromethyl)phenyl)methylamine in THF (13 ml) was then added, and stirring was carried out for a further 16 h at RT. The reaction solution was then washed three times with a sat. aq. NH₄Cl sol. and three times with a sat. aq. NaHCO₃ sol. The organic phase was dried over MgSO₄, filtered and concentrated in vacuo. After CC (EA) of the residue, 416 mg (0.85 mmol, 67%) of illustrative compound 24 were obtained. MS: m/z 482.2 [M+H]⁺.

### Synthesis of Illustrative Compound 34: (R)-3-Methyl-4-oxo-4-(4-phenyl-6,7-dihydrothieno[3,2-c]pyridin-5(4H)-yl)-N-(3-(trifluoromethyl)benzyl)butanamide

To a solution of 515 mg (1.56 mmol) of intermediate SAAS02 in THF (12 ml) there were added in succession 274 mg (1.56 mmol) of (3-trifluoromethyl)phenyl)methyl-amine, 594 mg (1.56 mmol) of HATU and 433 μl (3.13 mmol) of NEt₃. After 72 h stirring at RT, the reaction solution was diluted with EA and washed three times with a sat. aq. NH₄Cl sol. and three times with a sat. aq. NaHCO₃ sol. The organic phase was dried over MgSO₄, filtered and concentrated in vacuo. After CC (EA) of the residue, 550 mg (1.13 mmol, 72%) of illustrative compound 34 were obtained. MS: m/z 487.2 [M+H]⁺.

### Synthesis of Illustrative Compound 35: (−)-4-Oxo-4-(4-phenyl-6,7-dihydrothieno-[3,2-c]pyridin-5(4H)-yl)-N-(3-(trifluoromethyl)benzyl)butanamide and of Illustrative Compound 36: (+)-4-Oxo-4-(4-phenyl-6,7-dihydrothieno[3,2-c]pyridin-5(4H)-yl)-N-(3-(trifluoromethyl)benzyl)butanamide

210 mg of racemic 4-oxo-4-(4-phenyl-6,7-dihydrothieno[3,2-c]pyridin-5(4H)-yl)-N-(3-(trifluoromethyl)benzyl)-butanamide (for synthesis see Example 2) were separated into the two (R) and (S) enantiomers by means of chiral HPLC (column: Chiralpak AD-H, 250×4.6 mm, eluant: ethanol+0.1% diethylamine, flow rate 1 ml/min). 92 mg of illustrative compound 35, retention time 14.24 min, specific rotation: ^{[α]}^{_{D}}^{²⁵}-189.9° (MeOH, c=1.00), MS: m/z 473.1 [M+H]⁺ and 90 mg of illustrative compound 36, retention time 21.28 min, specific rotation:^{[α]}^{_{D}}^{²⁵}+194.3° (MeOH, c=1.00), MS: m/z 473.1 [M+H]⁺ were thereby obtained.

### Synthesis of Illustrative Compound 44: 4-Oxo-4-(5-phenyl-4,5-dihydrothieno-[2,3-c]pyridin-6(7H)-yl)-N-(3-(trifluoromethyl)benzyl)butanamide

1.71 g (8.9 mmol) of EDC, 797 mg (5.9 mmol) of HOBt and 4.0 ml (23.6 mmol) of DIPEA were added at 0° C. to a solution of 1.62 g (5.9 mmol) of intermediate PAAS01 in DCM (30 ml), and the mixture was then stirred for 20 min at 0° C. 1.27 g (5.9 mmol) of intermediate SAMN10 were added at that temperature, and stirring was then carried out for 16 h at RT. The mixture was then diluted with DCM and washed in succession with a sat. aq. NH₄Cl sol., brine, a sat. aq. Na₂CO₃ sol. and brine. The organic phase was dried over Na₂SO₄, filtered and concentrated in vacuo. After CC (hexane/EA 4:1) of the residue, 2.28 g (4.8 mmol, 82%) of illustrative compound 44 were obtained. MS: m/z 473.1 [M+H]⁺.

### Automated Synthesis of Illustrative Compounds 46 to 243

In a screw-cap jar having a magnetic stirring rod and a septum cap, a 0.105 M solution of CDI (105 μmol) in DCM (1 ml) was added to a 0.05 M solution of the particular carboxylic acid used (100 μmol) in DCM (2 ml). After 1 h stirring at RT, a 0.10 M solution of the particular amine used (100 μmol) in DCM (1 ml) was added by means of a pipette. Stirring was then carried out for a further 16 h at RT. Water (3 ml) was then added and the phases were mixed thoroughly for 30 min in a spin reactor. The magnetic stirring rod was separated off and the screw-cap jar was rinsed with DCM (2×1.25 ml). The phases were separated by removing the aqueous phase from the swelling vessel (Allex system from Mettler-Toledo). Further water (3 ml) was then added, the whole was mixed thoroughly, and the phases were separated as described. This procedure was repeated with brine (2.5 ml). The organic phase was then transferred to a test tube and concentrated in vacuo (evaporator from Genevac). The resulting residue was purified by means of HPLC.

### Synthesis of Further Illustrative Compounds

The synthesis of further illustrative compounds was carried out according to the processes already described. Table 2 indicates which compound was prepared by which process. It will be clear to persons skilled in the art which starting materials were used in each case.

TABLE 2

| | | Preparation | | MS |
|---|---|---|---|---|
| Illustrative | | analogous | Yield | m/z |
| compound | Chemical name | to example | [%] | [M + H]⁺ |
| 1 | 4-Oxo-4-(4-(thiophen-2-yl)-6,7- | 2 | 77 | 479.1 |
| | dihydrothieno[3,2-c]pyridin-5(4H)-yl)-N-(3- | | | |
| | (trifluoromethyl)benzyl)butanamide | | | |
| 4 | 4-Oxo-4-(4-m-tolyl-6,7-dihydrothieno[3,2-c]- | 9 | 67 | 487.2 |
| | pyridin-5(4H)-yl)-N-(3- | | | |
| | (trifluoromethyl)benzyl)butanamide | | | |
| 5 | 4-Oxo-4-(4-p-tolyl-6,7-dihydrothieno[3,2-c]- | 9 | 61 | 487.2 |
| | pyridin-5(4H)-yl)-N-(3- | | | |
| | (trifluoromethyl)benzyl)butanamide | | | |
| 6 | 4-(4-(2-Fluorophenyl)-6,7-dihydrothieno[3,2-c]- | 3 | 59 | 491.1 |
| | pyridin-5(4H)-yl)-4-oxo-N-(3- | | | |
| | (trifluoromethyl)benzyl)butanamide | | | |
| 7 | 4-(4-(4-Fluorophenyl)-6,7-dihydrothieno[3,2-c]- | 9 | 65 | 491.1 |
| | pyridin-5(4H)-yl)-4-oxo-N-(3- | | | |
| | (trifluoromethyl)benzyl)butanamide | | | |
| 8 | 4-Oxo-4-(4-o-tolyl-6,7-dihydrothieno[3,2-c]- | 9 | 46 | 487.2 |
| | pyridin-5(4H)-yl)-N-(3- | | | |
| | (trifluoromethyl)benzyl)butanamide | | | |
| 10 | 4-(2-Methyl-4-phenyl-6,7-dihydrothieno[3,2-c]- | 9 | 60 | 487.2 |
| | pyridin-5(4H)-yl)-4-oxo-N-(3- | | | |
| | (trifluoromethyl)benzyl)butanamide | | | |
| 13 | 4-Oxo-4-(4-phenyl-6,7-dihydrothieno[3,2-c]- | 12 | 56 | 473.1 |
| | pyridin-5(4H)-yl)-N-(4- | | | |
| | (trifluoromethyl)benzyl)butanamide | | | |
| 14 | N-(2-Methoxybenzyl)-4-oxo-4-(4-phenyl-6,7- | 12 | 61 | 435.2 |
| | dihydrothieno[3,2-c]pyridin-5(4H)- | | | |
| | yl)butanamide | | | |
| 15 | N-(3-Methoxybenzyl)-4-oxo-4-(4-phenyl-6,7- | 12 | 52 | 435.2 |
| | dihydrothieno[3,2-c]pyridin-5(4H)- | | | |
| | yl)butanamide | | | |
| 16 | N-(4-Methoxybenzyl)-4-oxo-4-(4-phenyl-6,7- | 12 | 82 | 435.2 |
| | dihydrothieno[3,2-c]pyridin-5(4H)- | | | |
| | yl)butanamide | | | |
| 17 | 4-Oxo-4-(4-phenyl-6,7-dihydrothieno[3,2-c]- | 12 | 33 | 473.1 |
| | pyridin-5(4H)-yl)-N-(2- | | | |
| | (trifluoromethyl)benzyl)butanamide | | | |
| 18 | N-(3-Fluorobenzyl)-4-oxo-4-(4-phenyl-6,7- | 12 | 53 | 423.1 |
| | dihydrothieno[3,2-c]pyridin-5(4H)- | | | |
| | yl)butanamide | | | |
| 19 | N-(3-Methylbenzyl)-4-oxo-4-(4-phenyl-6,7- | 12 | 49 | 419.2 |
| | dihydrothieno[3,2-c]pyridin-5(4H)- | | | |
| | yl)butanamide | | | |
| 20 | 4-(3-Methyl-4-phenyl-6,7-dihydrothieno[3,2-c]- | 9 | 23 | 487.2 |
| | pyridin-5(4H)-yl)-4-oxo-N-(3- | | | |
| | (trifluoromethyl)benzyl)butanamide | | | |
| 21 | 4-(4-Cyclohexyl-6,7-dihydrothieno[3,2-c]- | 9 | 42 | 479.2 |
| | pyridin-5(4H)-yl)-4-oxo-N-(3- | | | |
| | (trifluoromethyl)benzyl)butanamide | | | |
| 22 | 4-(4-Isopropyl-6,7-dihydrothieno[3,2-c]pyridin- | 9 | 58 | 439.2 |
| | 5(4H)-yl)-4-oxo-N-(3- | | | |
| | (trifluoromethyl)benzyl)butanamide | | | |
| 23 | 4-(4-Butyl-6,7-dihydrothieno[3,2-c]pyridin- | 2 | 59 | 453.2 |
| | 5(4H)-yl)-4-oxo-N-(3- | | | |
| | (trifluoromethyl)benzyl)butanamide | | | |
| 25 | 4-Oxo-N-phenethyl-4-(4-phenyl-6,7- | 12 | 59 | 419.2 |
| | dihydrothieno[3,2-c]pyridin-5(4H)- | | | |
| | yl)butanamide | | | |
| 26 | N-(2-Methylbenzyl)-4-oxo-4-(4-phenyl-6,7- | 12 | 57 | 419.2 |
| | dihydrothieno[3,2-c]pyridin-5(4H)- | | | |
| | yl)butanamide | | | |
| 27 | 4-Oxo-2-phenyl-4-(4-phenyl-6,7- | 9 | 55 | 549.2 |
| | dihydrothieno[3,2-c]pyridin-5(4H)-yl)-N-(3- | | | |
| | (trifluoromethyl)benzyl)butanamide | | | |
| 28 | (R)-2-Methyl-4-oxo-4-(4-phenyl-6,7- | 2 | 88 | 487.2 |
| | dihydrothieno[3,2-c]pyridin-5(4H)-yl)-N-(3- | | | |
| | (trifluoromethyl)benzyl)butanamide | | | |
| 29 | 4-Oxo-3-phenyl-4-(4-phenyl-6,7- | 2 | 68 | 549.2 |
| | dihydrothieno[3,2-c]pyridin-5(4H)-yl)-N-(3- | | | |
| | (trifluoromethyl)benzyl)butanamide | | | |
| 30 | 4-(6,7-Dihydrothieno[3,2-c]pyridin-5(4H)-yl)-4- | 2 | 65 | 473.1 |
| | oxo-3-phenyl-N-(3- | | | |
| | (trifluoromethyl)benzyl)butanamide | | | |
| 31 | rac. (1S,2R)-2-(4-Phenyl-4,5,6,7- | 2 | 61 | 527.2 |
| | tetrahydrothieno[3,2-c]pyridine-5-carbonyl)-N- | | | |
| | (3-(trifluoromethyl)benzyl)cyclohexane- | | | |
| | carboxamide | | | |
| 32 | rac. (1S)-2-(4-Phenyl-4,5,6,7- | 2 | 66 | 527.2 |
| | tetrahydrothieno[3,2-c]pyridine-5-carbonyl)-N- | | | |
| | (3-(trifluoromethyl)benzyl)cyclohexane- | | | |
| | carboxamide | | | |
| 33 | rac. (1S,2R)-2-(4-Phenyl-4,5,6,7- | 2 | 21 | 513.2 |
| | tetrahydrothieno[3,2-c]pyridine-5-carbonyl)-N- | | | |
| | (3-(trifluoromethyl)benzyl)cyclopentane- | | | |
| | carboxamide | | | |
| 37 | 4-(6,7-Dihydrothieno[3,2-c]pyridin-5(4H)-yl)-4- | 2 | 69 | 473.1 |
| | oxo-2-phenyl-N-(3- | | | |
| | (trifluoromethyl)benzyl)butanamide | | | |
| 38 | N-(3,5-Bis(trifluoromethyl)benzyl)-4-oxo-4-(4- | 34 | 89 | 541.1 |
| | phenyl-6,7-dihydrothieno[3,2-c]pyridin-5(4H)- | | | |
| | yl)butanamide | | | |
| 39 | N-(2-Fluoro-5-(trifluoromethyl)benzyl)-4-oxo-4- | 11 | 63 | 491.1 |
| | (4-phenyl-6,7-dihydrothieno[3,2-c]pyridin- | | | |
| | 5(4H)-yl)butanamide | | | |
| 40 | N-(2-Fluoro-3-(trifluoromethyl)benzyl)-4-oxo-4- | 34 | 38 | 491.1 |
| | (4-phenyl-6,7-dihydrothieno[3,2-c]pyridin- | | | |
| | 5(4H)-yl)butanamide | | | |
| 41 | N-(3-Fluoro-5-(trifluoromethyl)benzyl)-4-oxo-4- | 34 | 81 | 491.1 |
| | (4-phenyl-6,7-dihydrothieno[3,2-c]pyridin- | | | |
| | 5(4H)-yl)butanamide | | | |
| 42 | rac. (1S,2R)-2-(4-Phenyl-4,5,6,7- | 9 | 54 | 485.1 |
| | tetrahydrothieno[3,2-c]pyridine-5-carbonyl)-N- | | | |
| | (3-(trifluoromethyl)benzyl)cyclopropane- | | | |
| | carboxamide | | | |
| 43 | N-(3,4-Difluorobenzyl)-4-oxo-4-(4-phenyl-6,7- | 9 | 75 | 441.1 |
| | dihydrothieno[3,2-c]pyridin-5(4H)- | | | |
| | yl)butanamide | | | |
| 45 | 4-(1-Methyl-4-phenyl-6,7-dihydrothieno[3,4- | 44 | 42 | 487.2 |
| | c]pyridin-5(4H)-yl)-4-oxo-N-(3- | | | |
| | (trifluoromethyl)benzyl)butanamide | | | |
| 46 | N-(4-Methoxyphenyl)-4-oxo-4-(4-p-tolyl-6,7- | 46-243 | N/A | 435.2 |
| | dihydrothieno[3,2-c]pyridin-5(4H)- | | | |
| | yl)butanamide | | | |
| 47 | N-(1-Methyl-1H-indazol-6-yl)-4-oxo-4-(4-p- | 46-243 | N/A | 459.2 |
| | tolyl-6,7-dihydrothieno[3,2-c]pyridin-5(4H)- | | | |
| | yl)butanamide | | | |
| 48 | N-Benzyl-4-oxo-4-(4-p-tolyl-6,7-dihydrothieno- | 46-243 | N/A | 419.2 |
| | [3,2-c]pyridin-5(4H)-yl)butanamide | | | |
| 49 | 4-Oxo-N-phenethyl-4-(4-p-tolyl-6,7- | 46-243 | N/A | 433.2 |
| | dihydrothieno[3,2-c]pyridin-5(4H)- | | | |
| | yl)butanamide | | | |
| 50 | 4-Oxo-N-(pyridin-4-ylmethyl)-4-(4-p-tolyl-6,7- | 46-243 | N/A | 420.2 |
| | dihydrothieno[3,2-c]pyridin-5(4H)- | | | |
| | yl)butanamide | | | |
| 51 | 4-Oxo-N-(3-phenylpropyl)-4-(4-p-tolyl-6,7- | 46-243 | N/A | 447.2 |
| | dihydrothieno[3,2-c]pyridin-5(4H)- | | | |
| | yl)butanamide | | | |
| 52 | N-(Benzo[c][1,2,5]thiadiazol-4-yl)-4-oxo-4-(4- | 46-243 | N/A | 463.1 |
| | m-tolyl-6,7-dihydrothieno[3,2-c]pyridin-5(4H)- | | | |
| | yl)butanamide | | | |
| 53 | N-(1-Methyl-1H-indazol-6-yl)-4-oxo-4-(4-m- | 46-243 | N/A | 459.2 |
| | tolyl-6,7-dihydrothieno[3,2-c]pyridin-5(4H)- | | | |
| | yl)butanamide | | | |
| 54 | 4-Oxo-N-(pyridin-2-ylmethyl)-4-(4-m-tolyl-6,7- | 46-243 | N/A | 420.2 |
| | dihydrothieno[3,2-c]pyridin-5(4H)- | | | |
| | yl)butanamide | | | |
| 55 | 1-(4-Methylpiperazin-1-yl)-4-(4-m-tolyl-6,7- | 46-243 | N/A | 412.2 |
| | dihydrothieno[3,2-c]pyridin-5(4H)-yl)butane- | | | |
| | 1,4-dione | | | |
| 56 | 3-Methyl-4-oxo-4-(4-phenyl-6,7- | 46-243 | N/A | 420.2 |
| | dihydrothieno[3,2-c]pyridin-5(4H)-yl)-N- | | | |
| | (pyridin-2-ylmethyl)butanamide | | | |
| 57 | 4-Oxo-4-(4-phenyl-6,7-dihydrothieno[3,2- | 46-243 | N/A | 411.1 |
| | c]pyridin-5(4H)-yl)-N-(thiophen-2- | | | |
| | ylmethyl)butanamide | | | |
| 58 | N-(2-Chlorophenyl)-4-(4-(4-fluorophenyl)-6,7- | 46-243 | N/A | 443.1 |
| | dihydrothieno[3,2-c]pyridin-5(4H)-yl)-4- | | | |
| | oxobutanamide | | | |
| 59 | 4-(4-(4-Chlorophenyl)-6,7-dihydrothieno[3,2-c]- | 46-243 | N/A | 429.1 |
| | pyridin-5(4H)-yl)-N-(furan-2-ylmethyl)-4- | | | |
| | oxobutanamide | | | |
| 60 | 4-(4-(4-Chlorophenyl)-6,7-dihydrothieno- | 46-243 | N/A | 391.1 |
| | [3,2-c]pyridin-5(4H)-yl)-4-oxo-N-propyl- | | | |
| | butanamide | | | |
| 61 | N-(2-Chlorobenzyl)-4-oxo-4-(4-m-tolyl-6,7- | 46-243 | N/A | 453.1 |
| | dihydrothieno[3,2-c]pyridin-5(4H)- | | | |
| | yl)butanamide | | | |
| 62 | N-(2,4-Dichlorobenzyl)-4-(4-(3-fluorophenyl)- | 46-243 | N/A | 491.1 |
| | 6,7-dihydrothieno[3,2-c]pyridin-5(4H)-yl)-4- | | | |
| | oxobutanamide | | | |
| 63 | N-(4-Fluorobenzyl)-4-(4-(3-fluorophenyl)-6,7- | 46-243 | N/A | 441.1 |
| | dihydrothieno[3,2-c]pyridin-5(4H)-yl)-4- | | | |
| | oxobutanamide | | | |
| 64 | N-(3,4-Dichlorobenzyl)-3-methyl-4-oxo-4-(4- | 46-243 | N/A | 487.1 |
| | phenyl-6,7-dihydrothieno[3,2-c]pyridin-5(4H)- | | | |
| | yl)butanamide | | | |
| 65 | N-(2,5-Difluorobenzyl)-3-methyl-4-oxo-4-(4- | 46-243 | N/A | 455.2 |
| | phenyl-6,7-dihydrothieno[3,2-c]pyridin-5(4H)- | | | |
| | yl)butanamide | | | |
| 66 | 3-Methyl-4-oxo-4-(4-phenyl-6,7- | 46-243 | N/A | 487.2 |
| | dihydrothieno[3,2-c]pyridin-5(4H)-yl)-N-(3- | | | |
| | (trifluoromethyl)benzyl)butanamide | | | |
| 67 | 1-(4-(4-Chlorophenyl)-6,7-dihydrothieno[3,2-c]- | 46-243 | N/A | 419.1 |
| | pyridin-5(4H)-yl)-4-morpholinobutane-1,4- | | | |
| | dione | | | |
| 68 | 1-(4-(4-Chlorophenyl)-6,7-dihydrothieno- | 46-243 | N/A | 512.1 |
| | [3,2-c]pyridin-5(4H)-yl)-4-(4-(4-fluorophenyl)- | | | |
| | piperazin-1-yl)butane-1,4-dione | | | |
| 69 | 2-Methyl-N-(2-(5-methyl-1H-pyrazol-1-yl)ethyl)- | 46-243 | N/A | 437.2 |
| | 4-oxo-4-(4-phenyl-6,7-dihydrothieno[3,2- | | | |
| | c]pyridin-5(4H)-yl)butanamide | | | |
| 70 | N-(Naphthalen-1-ylmethyl)-4-oxo-4-(4-phenyl- | 46-243 | N/A | 455.2 |
| | 6,7-dihydrothieno[3,2-c]pyridin-5(4H)- | | | |
| | yl)butanamide | | | |
| 71 | 4-(4-(4-Fluorophenyl)-6,7-dihydrothieno- | 46-243 | N/A | 437.2 |
| | [3,2-c]pyridin-5(4H)-yl)-N-(4-methylbenzyl)-4- | | | |
| | oxobutanamide | | | |
| 72 | N-(Benzo[d][1,3]dioxol-5-ylmethyl)-4-(4-(4- | 46-243 | N/A | 467.1 |
| | fluorophenyl)-6,7-dihydrothieno[3,2-c]pyridin- | | | |
| | 5(4H)-yl)-4-oxobutanamide | | | |
| 73 | 4-(4-(4-Fluorophenyl)-6,7-dihydrothieno[3,2-c]- | 46-243 | N/A | 491.1 |
| | pyridin-5(4H)-yl)-4-oxo-N-(2-(trifluoromethyl)- | | | |
| | benzyl)butanamide | | | |
| 74 | 4-(4-(4-Fluorophenyl)-6,7-dihydrothieno[3,2-c]- | 46-243 | N/A | 473.2 |
| | pyridin-5(4H)-yl)-N-(naphthalen-1-ylmethyl)-4- | | | |
| | oxobutanamide | | | |
| 75 | 4-(4-(4-Chlorophenyl)-6,7-dihydrothieno[3,2-c]- | 46-243 | N/A | 457.1 |
| | pyridin-5(4H)-yl)-N-(3-fluorobenzyl)-4- | | | |
| | oxobutanamide | | | |
| 76 | 2-Methyl-N-(1-methyl-1H-indazol-6-yl)-4-oxo-4- | 46-243 | N/A | 459.2 |
| | (4-phenyl-6,7-dihydrothieno[3,2-c]pyridin- | | | |
| | 5(4H)-yl)butanamide | | | |
| 77 | N-(4-Methoxybenzyl)-2-methyl-4-oxo-4-(4- | 46-243 | N/A | 449.2 |
| | phenyl-6,7-dihydrothieno[3,2-c]pyridin-5(4H)- | | | |
| | yl)butanamide | | | |
| 78 | 2-Methyl-1-(4-methylpiperazin-1-yl)-4-(4- | 46-243 | N/A | 412.2 |
| | phenyl-6,7-dihydrothieno[3,2-c]pyridin-5(4H)- | | | |
| | yl)butane-1,4-dione | | | |
| 79 | N-(2-(1H-Indol-3-yl)ethyl)-2-methyl-4-oxo-4-(4- | 46-243 | N/A | 472.2 |
| | phenyl-6,7-dihydrothieno[3,2-c]pyridin-5(4H)- | | | |
| | yl)butanamide | | | |
| 80 | N-(2-Fluorobenzyl)-2-methyl-4-oxo-4-(4- | 46-243 | N/A | 437.2 |
| | phenyl-6,7-dihydrothieno[3,2-c]pyridin-5(4H)- | | | |
| | yl)butanamide | | | |
| 81 | 2-Methyl-4-oxo-4-(4-phenyl-6,7- | 46-243 | N/A | 487.2 |
| | dihydrothieno[3,2-c]pyridin-5(4H)-yl)-N-(2- | | | |
| | (trifluoromethyl)benzyl)butanamide | | | |
| 82 | 4-Oxo-N-(2-(piperidin-1-yl)ethyl)-4-(4-p-tolyl- | 46-243 | N/A | 440.2 |
| | 6,7-dihydrothieno[3,2-c]pyridin-5(4H)- | | | |
| | yl)butanamide | | | |
| 83 | 4-Oxo-N-((tetrahydrofuran-2-yl)methyl)-4-(4-p- | 46-243 | N/A | 413.2 |
| | tolyl-6,7-dihydrothieno[3,2-c]pyridin-5(4H)- | | | |
| | yl)butanamide | | | |
| 84 | N-(4-Chlorobenzyl)-4-oxo-4-(4-p-tolyl-6,7- | 46-243 | N/A | 453.1 |
| | dihydrothieno[3,2-c]pyridin-5(4H)- | | | |
| | yl)butanamide | | | |
| 85 | N-(2,3-Dichlorobenzyl)-4-oxo-4-(4-p-tolyl-6,7- | 46-243 | N/A | 487.1 |
| | dihydrothieno[3,2-c]pyridin-5(4H)- | | | |
| | yl)butanamide | | | |
| 86 | 4-Oxo-N-(2-(thiophen-2-yl)ethyl)-4-(4-m-tolyl- | 46-243 | N/A | 439.1 |
| | 6,7-dihydrothieno[3,2-c]pyridin-5(4H)- | | | |
| | yl)butanamide | | | |
| 87 | N-(Cyclohexylmethyl)-4-oxo-4-(4-m-tolyl-6,7- | 46-243 | N/A | 425.2 |
| | dihydrothieno[3,2-c]pyridin-5(4H)- | | | |
| | yl)butanamide | | | |
| 88 | N-(3-Chlorophenethyl)-4-oxo-4-(4-m-tolyl-6,7- | 46-243 | N/A | 467.1 |
| | dihydrothieno[3,2-c]pyridin-5(4H)- | | | |
| | yl)butanamide | | | |
| 89 | N-(3,3-Diphenylpropyl)-4-oxo-4-(4-m-tolyl-6,7- | 46-243 | N/A | 523.2 |
| | dihydrothieno[3,2-c]pyridin-5(4H)- | | | |
| | yl)butanamide | | | |
| 90 | 4-(4-(3-Fluorophenyl)-6,7-dihydrothieno[3,2- | 46-243 | N/A | 460.2 |
| | c]pyridin-5(4H)-yl)-N-(3-morpholinopropyl)-4- | | | |
| | oxobutanamide | | | |
| 91 | 4-(4-(3-Fluorophenyl)-6,7-dihydrothieno[3,2- | 46-243 | N/A | 424.1 |
| | c]pyridin-5(4H)-yl)-4-oxo-N-(pyridin-3- | | | |
| | ylmethyl)butanamide | | | |
| 92 | 4-(4-(3-Fluorophenyl)-6,7-dihydrothieno[3,2- | 46-243 | N/A | 437.2 |
| | c]pyridin-5(4H)-yl)-N-(3-methylbenzyl)-4- | | | |
| | oxobutanamide | | | |
| 93 | 4-(4-(3-Fluorophenyl)-6,7-dihydrothieno[3,2- | 46-243 | N/A | 451.2 |
| | c]pyridin-5(4H)-yl)-N-(4-methylphenethyl)-4- | | | |
| | oxobutanamide | | | |
| 94 | 3-Methyl-4-oxo-4-(4-phenyl-6,7- | 46-243 | N/A | 461.2 |
| | dihydrothieno[3,2-c]pyridin-5(4H)-yl)-N-(4- | | | |
| | phenylbutyl)butanamide | | | |
| 95 | N-(Biphenyl-4-ylmethyl)-4-oxo-4-(4-m-tolyl-6,7- | 46-243 | N/A | 495.2 |
| | dihydrothieno[3,2-c]pyridin-5(4H)- | | | |
| | yl)butanamide | | | |
| 96 | 4-(4-(3-Fluorophenyl)-6,7-dihydrothieno[3,2-c]- | 46-243 | N/A | 453.2 |
| | pyridin-5(4H)-yl)-N-(2-methoxybenzyl)-4- | | | |
| | oxobutanamide | | | |
| 97 | N-(4-Chlorophenethyl)-4-(4-(3-fluorophenyl)- | 46-243 | N/A | 471.1 |
| | 6,7-dihydrothieno[3,2-c]pyridin-5(4H)-yl)-4- | | | |
| | oxobutanamide | | | |
| 98 | 4-(4-(3-Fluorophenyl)-6,7-dihydrothieno[3,2- | 46-243 | N/A | 504.2 |
| | c]pyridin-5(4H)-yl)-N-((5-methyl-3- | | | |
| | phenylisoxazol-4-yl)methyl)-4-oxobutanamide | | | |
| 99 | N-(2,6-Difluorobenzyl)-3-methyl-4-oxo-4-(4- | 46-243 | N/A | 455.2 |
| | phenyl-6,7-dihydrothieno[3,2-c]pyridin-5(4H)- | | | |
| | yl)butanamide | | | |
| 100 | N-(3,5-Difluorobenzyl)-3-methyl-4-oxo-4-(4- | 46-243 | N/A | 455.2 |
| | phenyl-6,7-dihydrothieno[3,2-c]pyridin-5(4H)- | | | |
| | yl)butanamide | | | |
| 101 | N-(3-Chlorobenzyl)-3-methyl-4-oxo-4-(4- | 46-243 | N/A | 453.1 |
| | phenyl-6,7-dihydrothieno[3,2-c]pyridin-5(4H)- | | | |
| | yl)butanamide | | | |
| 102 | N-(3,5-Dimethoxyphenethyl)-3-methyl-4-oxo-4- | 46-243 | N/A | 493.2 |
| | (4-phenyl-6,7-dihydrothieno[3,2-c]pyridin- | | | |
| | 5(4H)-yl)butanamide | | | |
| 103 | N-(3,4-Difluorobenzyl)-4-oxo-4-(4-phenyl-6,7- | 46-243 | N/A | 441.1 |
| | dihydrothieno[3,2-c]pyridin-5(4H)- | | | |
| | yl)butanamide | | | |
| 104 | N-(2,4-Dichlorophenethyl)-4-oxo-4-(4-phenyl- | 46-243 | N/A | 487.1 |
| | 6,7-dihydrothieno[3,2-c]pyridin-5(4H)- | | | |
| | yl)butanamide | | | |
| 105 | N-(2-(1H-1,2,4-Triazol-1-yl)ethyl)-4-oxo-4-(4- | 46-243 | N/A | 410.2 |
| | phenyl-6,7-dihydrothieno[3,2-c]pyridin-5(4H)- | | | |
| | yl)butanamide | | | |
| 106 | 4-(4-(4-Fluorophenyl)-6,7-dihydrothieno[3,2-c]- | 46-243 | N/A | 437.2 |
| | pyridin-5(4H)-yl)-N-(2-methylbenzyl)-4- | | | |
| | oxobutanamide | | | |
| 107 | N-(4-Fluorophenethyl)-4-(4-(4-fluorophenyl)- | 46-243 | N/A | 455.2 |
| | 6,7-dihydrothieno[3,2-c]pyridin-5(4H)-yl)-4- | | | |
| | oxobutanamide | | | |
| 108 | 4-(4-(4-Fluorophenyl)-6,7-dihydrothieno[3,2-c]- | 46-243 | N/A | 428.1 |
| | pyridin-5(4H)-yl)-N-((5-methylisoxazol-3- | | | |
| | yl)methyl)-4-oxobutanamide | | | |
| 109 | N-(2-Chlorophenethyl)-4-(4-(4-fluorophenyl)- | 46-243 | N/A | 471.1 |
| | 6,7-dihydrothieno[3,2-c]pyridin-5(4H)-yl)-4- | | | |
| | oxobutanamide | | | |
| 110 | 4-(4-(4-Chlorophenyl)-6,7-dihydrothieno[3,2-c]- | 46-243 | N/A | 457.2 |
| | pyridin-5(4H)-yl)-N-(2-cyclohexenylethyl)-4- | | | |
| | oxobutanamide | | | |
| 111 | 4-(4-(4-Chlorophenyl)-6,7-dihydrothieno[3,2-c]- | 46-243 | N/A | 499.1 |
| | pyridin-5(4H)-yl)-N-(3,5-dimethoxybenzyl)-4- | | | |
| | oxobutanamide | | | |
| 112 | 4-(4-(4-Chlorophenyl)-6,7-dihydrothieno[3,2-c]- | 46-243 | N/A | 521.1 |
| | pyridin-5(4H)-yl)-N-(3,4-dichlorophenethyl)-4- | | | |
| | oxobutanamide | | | |
| 113 | 4-(4-(4-Chlorophenyl)-6,7-dihydrothieno[3,2-c]- | 46-243 | N/A | 471.1 |
| | pyridin-5(4H)-yl)-N-(3-fluorophenethyl)-4- | | | |
| | oxobutanamide | | | |
| 114 | 1-(3-Phenylpiperidin-1-yl)-4-(4-p-tolyl-6,7- | 46-243 | N/A | 473.2 |
| | dihydrothieno[3,2-c]pyridin-5(4H)-yl)butane- | | | |
| | 1,4-dione | | | |
| 115 | 1-(4-Benzylpiperazin-1-yl)-4-(4-p-tolyl-6,7- | 46-243 | N/A | 488.2 |
| | dihydrothieno[3,2-c]pyridin-5(4H)-yl)butane- | | | |
| | 1,4-dione | | | |
| 116 | 1-(4-(4-Methoxyphenyl)piperazin-1-yl)-4-(4-p- | 46-243 | N/A | 504.2 |
| | tolyl-6,7-dihydrothieno[3,2-c]pyridin-5(4H)- | | | |
| | yl)butane-1,4-dione | | | |
| 117 | 1-(2-Benzylpiperidin-1-yl)-4-(4-m-tolyl-6,7- | 46-243 | N/A | 487.2 |
| | dihydrothieno[3,2-c]pyridin-5(4H)-yl)butane- | | | |
| | 1,4-dione | | | |
| 118 | 1-(4-(2-Fluorophenyl)piperazin-1-yl)-4-(4-m- | 46-243 | N/A | 492.2 |
| | tolyl-6,7-dihydrothieno[3,2-c]pyridin-5(4H)- | | | |
| | yl)butane-1,4-dione | | | |
| 119 | 1-(4-(Cyclohexylmethyl)piperazin-1-yl)-4-(4-m- | 46-243 | N/A | 494.3 |
| | tolyl-6,7-dihydrothieno[3,2-c]pyridin-5(4H)- | | | |
| | yl)butane-1,4-dione | | | |
| 120 | N-(3-(1H-Imidazol-1-yl)propyl)-4-(4-(3- | 46-243 | N/A | 441.2 |
| | fluorophenyl)-6,7-dihydrothieno[3,2-c]pyridin- | | | |
| | 5(4H)-yl)-4-oxobutanamide | | | |
| 121 | 1-(4-(3-Fluorophenyl)-6,7-dihydrothieno[3,2-c]- | 46-243 | N/A | 463.2 |
| | pyridin-5(4H)-yl)-4-(3-phenylpyrrolidin-1- | | | |
| | yl)butane-1,4-dione | | | |
| 122 | 1-(4-(3-Fluorophenyl)-6,7-dihydrothieno[3,2-c]- | 46-243 | N/A | 496.2 |
| | pyridin-5(4H)-yl)-4-(4-(furan-2- | | | |
| | carbonyl)piperazin-1-yl)butane-1,4-dione | | | |
| 123 | N-(3-(3,4-Dihydroquinolin-1(2H)-yl)propyl)-3- | 46-243 | N/A | 502.2 |
| | methyl-4-oxo-4-(4-phenyl-6,7- | | | |
| | dihydrothieno[3,2-c]pyridin-5(4H)- | | | |
| | yl)butanamide | | | |
| 124 | 2-Methyl-1-(4-phenyl-6,7-dihydrothieno[3,2-c]- | 46-243 | N/A | 397.2 |
| | pyridin-5(4H)-yl)-4-(piperidin-1-yl)butane-1,4- | | | |
| | dione | | | |
| 125 | 4-(4-(2-Methoxyphenyl)piperazin-1-yl)-2- | 46-243 | N/A | 504.2 |
| | methyl-1-(4-phenyl-6,7-dihydrothieno[3,2-c]- | | | |
| | pyridin-5(4H)-yl)butane-1,4-dione | | | |
| 126 | 4-Oxo-4-(4-phenyl-6,7-dihydrothieno[3,2-c]- | 46-243 | N/A | 421.2 |
| | pyridin-5(4H)-yl)-N-(2-(pyrazin-2- | | | |
| | yl)ethyl)butanamide | | | |
| 127 | N,N-Diethyl-4-oxo-4-(4-phenyl-6,7- | 46-243 | N/A | 371.2 |
| | dihydrothieno[3,2-c]pyridin-5(4H)- | | | |
| | yl)butanamide | | | |
| 128 | 1-(4-Phenyl-6,7-dihydrothieno[3,2-c]pyridin- | 46-243 | N/A | 461.2 |
| | 5(4H)-yl)-4-(4-(pyridin-2-yl)piperazin-1- | | | |
| | yl)butane-1,4-dione | | | |
| 129 | 1-(4-(4-Fluorophenyl)-6,7-dihydrothieno[3,2-c]- | 46-243 | N/A | 508.2 |
| | pyridin-5(4H)-yl)-4-(4-(3-methoxyphenyl)- | | | |
| | piperazin-1-yl)butane-1,4-dione | | | |
| 130 | 1-(4-Isopropylpiperazin-1-yl)-4-(4-p-tolyl-6,7- | 46-243 | N/A | 440.2 |
| | dihydrothieno[3,2-c]pyridin-5(4H)-yl)butane- | | | |
| | 1,4-dione | | | |
| 131 | 1-(4-p-Tolyl-6,7-dihydrothieno[3,2-c]pyridin- | 46-243 | N/A | 542.2 |
| | 5(4H)-yl)-4-(4-(3-(trifluoromethyl)- | | | |
| | phenyl)piperazin-1-yl)butane-1,4-dione | | | |
| 132 | 1-(4-Phenylpiperazin-1-yl)-4-(4-m-tolyl-6,7- | 46-243 | N/A | 474.2 |
| | dihydrothieno[3,2-c]pyridin-5(4H)-yl)butane- | | | |
| | 1,4-dione | | | |
| 133 | 1-(3,5-Dimethylpiperidin-1-yl)-4-(4-m-tolyl-6,7- | 46-243 | N/A | 425.2 |
| | dihydrothieno[3,2-c]pyridin-5(4H)-yl)butane- | | | |
| | 1,4-dione | | | |
| 134 | 1-(5,6-Dihydropyridin-1(2H)-yl)-4-(4-(3- | 46-243 | N/A | 399.1 |
| | fluorophenyl)-6,7-dihydrothieno[3,2-c]pyridin- | | | |
| | 5(4H)-yl)butane-1,4-dione | | | |
| 135 | 1-(4-(3-Fluorophenyl)-6,7-dihydrothieno[3,2-c]- | 46-243 | N/A | 480.2 |
| | pyridin-5(4H)-yl)-4-(4-(pyrimidin-2-yl)piperazin- | | | |
| | 1-yl)butane-1,4-dione | | | |
| 136 | 1-(4-(3-Fluorophenyl)-6,7-dihydrothieno[3,2-c]- | 46-243 | N/A | 415.2 |
| | pyridin-5(4H)-yl)-4-(3-methylpiperidin-1- | | | |
| | yl)butane-1,4-dione | | | |
| 137 | N-Ethyl-4-(4-(3-fluorophenyl)-6,7- | 46-243 | N/A | 452.2 |
| | dihydrothieno[3,2-c]pyridin-5(4H)-yl)-4-oxo-N- | | | |
| | (pyridin-4-ylmethyl)butanamide | | | |
| 138 | 4-(4-Acetylpiperazin-1-yl)-2-methyl-1-(4- | 46-243 | N/A | 440.2 |
| | phenyl-6,7-dihydrothieno[3,2-c]pyridin-5(4H)- | | | |
| | yl)butane-1,4-dione | | | |
| 139 | 4-(2,6-Dimethylmorpholino)-2-methyl-1-(4- | 46-243 | N/A | 427.2 |
| | phenyl-6,7-dihydrothieno[3,2-c]pyridin-5(4H)- | | | |
| | yl)butane-1,4-dione | | | |
| 140 | 4-(2,5-Dihydro-1H-pyrrol-1-yl)-2-methyl-1-(4- | 46-243 | N/A | 381.2 |
| | phenyl-6,7-dihydrothieno[3,2-c]pyridin-5(4H)- | | | |
| | yl)butane-1,4-dione | | | |
| 141 | N-(2-Methoxyphenethyl)-4-oxo-4-(4-phenyl- | 46-243 | N/A | 449.2 |
| | 6,7-dihydrothieno[3,2-c]pyridin-5(4H)- | | | |
| | yl)butanamide | | | |
| 142 | 4-(4-(4-Fluorophenyl)-6,7-dihydrothieno[3,2- | 46-243 | N/A | 403.2 |
| | c]pyridin-5(4H)-yl)-N-isopentyl-4- | | | |
| | oxobutanamide | | | |
| 143 | N-(2,4-Dimethoxybenzyl)-4-(4-(4-fluorophenyl)- | 46-243 | N/A | 483.2 |
| | 6,7-dihydrothieno[3,2-c]pyridin-5(4H)-yl)-4- | | | |
| | oxobutanamide | | | |
| 144 | 4-(4-(4-Chlorophenyl)-6,7-dihydrothieno[3,2-c]- | 46-243 | N/A | 443.1 |
| | pyridin-5(4H)-yl)-N-((5-methylfuran-2- | | | |
| | yl)methyl)-4-oxobutanamide | | | |
| 145 | 4-(4-(4-Chlorophenyl)-6,7-dihydrothieno[3,2-c]- | 46-243 | N/A | 419.1 |
| | pyridin-5(4H)-yl)-4-oxo-N-pentylbutanamide | | | |
| 146 | 1-(4-Benzylpiperidin-1-yl)-4-(4-(4- | 46-243 | N/A | 507.2 |
| | chlorophenyl)-6,7-dihydrothieno[3,2-c]pyridin- | | | |
| | 5(4H)-yl)butane-1,4-dione | | | |
| 147 | 1-(4-(4-Chlorophenyl)-6,7-dihydrothieno[3,2-c]- | 46-243 | N/A | 431.1 |
| | pyridin-5(4H)-yl)-4-(4-methylpiperidin-1- | | | |
| | yl)butane-1,4-dione | | | |
| 148 | 1-(Azetidin-1-yl)-4-(4-(4-chlorophenyl)-6,7- | 46-243 | N/A | 389.1 |
| | dihydrothieno[3,2-c]pyridin-5(4H)-yl)butane- | | | |
| | 1,4-dione | | | |
| 149 | 1-(3,4-Dihydroisoquinolin-2(1H)-yl)-2-methyl-4- | 46-243 | N/A | 445.2 |
| | (4-phenyl-6,7-dihydrothieno[3,2-c]pyridin- | | | |
| | 5(4H)-yl)butane-1,4-dione | | | |
| 150 | 1-(4-(Benzo[d][1,3]dioxol-5-ylmethyl)piperazin- | 46-243 | N/A | 532.2 |
| | 1-yl)-2-methyl-4-(4-phenyl-6,7- | | | |
| | dihydrothieno[3,2-c]pyridin-5(4H)-yl)butane- | | | |
| | 1,4-dione | | | |
| 151 | N-Benzyl-N-ethyl-4-oxo-4-(4-p-tolyl-6,7- | 46-243 | N/A | 447.2 |
| | dihydrothieno[3,2-c]pyridin-5(4H)- | | | |
| | yl)butanamide | | | |
| 152 | N-Methyl-4-oxo-N-phenethyl-4-(4-p-tolyl-6,7- | 46-243 | N/A | 447.2 |
| | dihydrothieno[3,2-c]pyridin-5(4H)- | | | |
| | yl)butanamide | | | |
| 153 | Ethyl 1-(4-oxo-4-(4-p-tolyl-6,7- | 46-243 | N/A | 469.2 |
| | dihydrothieno[3,2-c]pyridin-5(4H)- | | | |
| | yl)butanoyl)piperidine-4-carboxylate | | | |
| 154 | (E)-1-(4-(3-Phenylprop-2-enyl)-piperazin-1-yl)- | 46-243 | N/A | 514.2 |
| | 4-(4-p-tolyl-6,7-dihydrothieno[3,2-c]pyridin- | | | |
| | 5(4H)-yl)butane-1,4-dione | | | |
| 155 | 1-(2-(Thiazol-2-yl)pyrrolidin-1-yl)-4-(4-p-tolyl- | 46-243 | N/A | 466.2 |
| | 6,7-dihydrothieno[3,2-c]pyridin-5(4H)- | | | |
| | yl)butane-1,4-dione | | | |
| 156 | N-Cyclohexyl-N-ethyl-4-oxo-4-(4-m-tolyl-6,7- | 46-243 | N/A | 439.2 |
| | dihydrothieno[3,2-c]pyridin-5(4H)- | | | |
| | yl)butanamide | | | |
| 157 | N-Ethyl-N-isopropyl-4-oxo-4-(4-m-tolyl-6,7- | 46-243 | N/A | 399.2 |
| | dihydrothieno[3,2-c]pyridin-5(4H)- | | | |
| | yl)butanamide | | | |
| 158 | 1-(Pyrrolidin-1-yl)-4-(4-m-tolyl-6,7- | 46-243 | N/A | 383.2 |
| | dihydrothieno[3,2-c]pyridin-5(4H)-yl)butane- | | | |
| | 1,4-dione | | | |
| 159 | 1-(4-(Pyridin-4-yl)piperazin-1-yl)-4-(4-m-tolyl- | 46-243 | N/A | 475.2 |
| | 6,7-dihydrothieno[3,2-c]pyridin-5(4H)- | | | |
| | yl)butane-1,4-dione | | | |
| 160 | 1-(2-(Pyridin-2-ylmethyl)pyrrolidin-1-yl)-4-(4-m- | 46-243 | N/A | 474.2 |
| | tolyl-6,7-dihydrothieno[3,2-c]pyridin-5(4H)- | | | |
| | yl)butane-1,4-dione | | | |
| 161 | Ethyl 1-(4-(4-(4-fluorophenyl)-6,7- | 46-243 | N/A | 473.2 |
| | dihydrothieno[3,2-c]pyridin-5(4H)-yl)-4- | | | |
| | oxobutanoyl)piperidine-3-carboxylate | | | |
| 162 | 1-(4-(4-Fluorophenyl)-6,7-dihydrothieno[3,2-c]- | 46-243 | N/A | 546.2 |
| | pyridin-5(4H)-yl)-4-(4-(4-(trifluoromethyl)- | | | |
| | phenyl)piperazin-1-yl)butane-1,4-dione | | | |
| 163 | 1-(2-(5-Bromopyridin-3-yl)pyrrolidin-1-yl)-4-(4- | 46-243 | N/A | 542.1 |
| | (4-fluorophenyl)-6,7-dihydrothieno[3,2-c]- | | | |
| | pyridin-5(4H)-yl)butane-1,4-dione | | | |
| 164 | 1-(4-Ethylpiperazin-1-yl)-4-(4-phenyl-6,7- | 46-243 | N/A | 412.2 |
| | dihydrothieno[3,2-c]pyridin-5(4H)-yl)butane- | | | |
| | 1,4-dione | | | |
| 165 | 1-(2-Ethylpiperidin-1-yl)-4-(4-phenyl-6,7- | 46-243 | N/A | 411.2 |
| | dihydrothieno[3,2-c]pyridin-5(4H)-yl)butane- | | | |
| | 1,4-dione | | | |
| 166 | N,N-Dibenzyl-4-oxo-4-(4-phenyl-6,7- | 46-243 | N/A | 495.2 |
| | dihydrothieno[3,2-c]pyridin-5(4H)- | | | |
| | yl)butanamide | | | |
| 167 | N,N-Diisobutyl-4-oxo-4-(4-phenyl-6,7- | 46-243 | N/A | 427.2 |
| | dihydrothieno[3,2-c]pyridin-5(4H)- | | | |
| | yl)butanamide | | | |
| 168 | 1-(3,4-Dihydroquinolin-1(2H)-yl)-4-(4-(4- | 46-243 | N/A | 449.2 |
| | fluorophenyl)-6,7-dihydrothieno[3,2-c]pyridin- | | | |
| | 5(4H)-yl)butane-1,4-dione | | | |
| 169 | 1-(4-(3,4-Dichlorobenzyl)piperazin-1-yl)-4-(4-p- | 46-243 | N/A | 556.2 |
| | tolyl-6,7-dihydrothieno[3,2-c]pyridin-5(4H)- | | | |
| | yl)butane-1,4-dione | | | |
| 170 | 1-(4-p-Tolyl-6,7-dihydrothieno[3,2-c]pyridin- | 46-243 | N/A | 530.3 |
| | 5(4H)-yl)-4-(4-(2,4,6-trimethylbenzyl)piperazin- | | | |
| | 1-yl)butane-1,4-dione | | | |
| 171 | 1-(4-(4-Bromobenzyl)piperazin-1-yl)-4-(4-m- | 46-243 | N/A | 566.1 |
| | tolyl-6,7-dihydrothieno[3,2-c]pyridin-5(4H)- | | | |
| | yl)butane-1,4-dione | | | |
| 172 | 1-(4-(4-Chlorobenzyl)piperazin-1-yl)-4-(4-m- | 46-243 | N/A | 522.2 |
| | tolyl-6,7-dihydrothieno[3,2-c]pyridin-5(4H)- | | | |
| | yl)butane-1,4-dione | | | |
| 173 | 1-(2-((4,6-Dimethylpyridin-2- | 46-243 | N/A | 506.2 |
| | yl)methyl)pyrrolidin-1-yl)-4-(4-(3-fluorophenyl)- | | | |
| | 6,7-dihydrothieno[3,2-c]pyridin-5(4H)- | | | |
| | yl)butane-1,4-dione | | | |
| 174 | 1-(4-(3-Fluorophenyl)-6,7-dihydrothieno[3,2-c]- | 46-243 | N/A | 492.2 |
| | pyridin-5(4H)-yl)-4-(2-((6-methylpyridin-2- | | | |
| | yl)methyl)pyrrolidin-1-yl)butane-1,4-dione | | | |
| 175 | 1-(2-((5-Ethylpyridin-2-yl)methyl)pyrrolidin-1- | 46-243 | N/A | 506.2 |
| | yl)-4-(4-(3-fluorophenyl)-6,7-dihydrothieno- | | | |
| | [3,2-c]pyridin-5(4H)-yl)butane-1,4-dione | | | |
| 176 | 1-(4-(3-Fluorophenyl)-6,7-dihydrothieno[3,2-c]- | 46-243 | N/A | 508.2 |
| | pyridin-5(4H)-yl)-4-(2-(6-methoxypyridin-3- | | | |
| | yl)piperidin-1-yl)butane-1,4-dione | | | |
| 177 | 4-(4-(3-Fluorophenyl)-6,7-dihydrothieno[3,2-c]- | 46-243 | N/A | 438.2 |
| | pyridin-5(4H)-yl)-N-methyl-4-oxo-N-(pyridin-3- | | | |
| | ylmethyl)butanamide | | | |
| 178 | N-(2,2-Diphenylethyl)-3-methyl-4-oxo-4-(4- | 46-243 | N/A | 509.2 |
| | phenyl-6,7-dihydrothieno[3,2-c]pyridin-5(4H)- | | | |
| | yl)butanamide | | | |
| 179 | N-(Cyclopropylmethyl)-4-oxo-4-(4-phenyl-6,7- | 46-243 | N/A | 369.2 |
| | dihydrothieno[3,2-c]pyridin-5(4H)- | | | |
| | yl)butanamide | | | |
| 180 | 1-(4-(3-Fluorophenyl)-6,7-dihydrothieno[3,2-c]- | 46-243 | N/A | 506.2 |
| | pyridin-5(4H)-yl)-4-(4-(4-methylbenzyl)- | | | |
| | piperazin-1-yl)butane-1,4-dione | | | |
| 181 | 2-Methyl-1-(4-phenyl-6,7-dihydrothieno[3,2-c]- | 46-243 | N/A | 488.2 |
| | pyridin-5(4H)-yl)-4-(2-(pyridin-2-ylmethyl)- | | | |
| | piperidin-1-yl)butane-1,4-dione | | | |
| 182 | 4-(4-(3,5-Dichloropyridin-4-yl)piperazin-1-yl)-2- | 46-243 | N/A | 543.1 |
| | methyl-1-(4-phenyl-6,7-dihydrothieno[3,2-c]- | | | |
| | pyridin-5(4H)-yl)butane-1,4-dione | | | |
| 183 | 1-(2-((4,6-Dimethylpyridin-2-yl)methyl)- | 46-243 | N/A | 502.2 |
| | piperidin-1-yl)-4-(4-phenyl-6,7-dihydrothieno- | | | |
| | [3,2-c]pyridin-5(4H)-yl)butane-1,4-dione | | | |
| 184 | N-(4-Fluorobenzyl)-N-methyl-4-oxo-4-(4- | 46-243 | N/A | 437.2 |
| | phenyl-6,7-dihydrothieno[3,2-c]pyridin-5(4H)- | | | |
| | yl)butanamide | | | |
| 185 | 1-(4-(3-Fluorophenyl)-6,7-dihydrothieno[3,2- | 46-243 | N/A | 464.2 |
| | c]pyridin-5(4H)-yl)-4-(2-(pyridin-2-yl)pyrrolidin- | | | |
| | 1-yl)butane-1,4-dione | | | |
| 186 | 4-(4-(4-Methoxybenzyl)piperazin-1-yl)-2- | 46-243 | N/A | 518.2 |
| | methyl-1-(4-phenyl-6,7-dihydrothieno[3,2- | | | |
| | c]pyridin-5(4H)-yl)butane-1,4-dione | | | |
| 187 | 2-Methyl-1-(4-phenyl-6,7-dihydrothieno[3,2-c]- | 46-243 | N/A | 460.2 |
| | pyridin-5(4H)-yl)-4-(3-(pyridin-3-yl)pyrrolidin-1- | | | |
| | yl)butane-1,4-dione | | | |
| 188 | 1-(4-(2-Fluorobenzyl)piperazin-1-yl)-4-(4- | 46-243 | N/A | 492.2 |
| | phenyl-6,7-dihydrothieno[3,2-c]pyridin-5(4H)- | | | |
| | yl)butane-1,4-dione | | | |
| 189 | N-Methyl-4-oxo-4-(4-phenyl-6,7-dihydrothieno- | 46-243 | N/A | 434.2 |
| | [3,2-c]pyridin-5(4H)-yl)-N-(2-(pyridin-4- | | | |
| | yl)ethyl)butanamide | | | |
| 190 | N-Butyl-N-ethyl-3-methyl-4-oxo-4-(4-phenyl- | 46-243 | N/A | 413.2 |
| | 6,7-dihydrothieno[3,2-c]pyridin-5(4H)- | | | |
| | yl)butanamide | | | |
| 191 | N,3-Dimethyl-4-oxo-4-(4-phenyl-6,7- | 46-243 | N/A | 385.2 |
| | dihydrothieno[3,2-c]pyridin-5(4H)-yl)-N- | | | |
| | propylbutanamide | | | |
| 192 | 4-((S)-2-(Methoxymethyl)pyrrolidin-1-yl)-2- | 46-243 | N/A | 427.2 |
| | methyl-1-(4-phenyl-6,7-dihydrothieno[3,2-c]- | | | |
| | pyridin-5(4H)-yl)butane-1,4-dione | | | |
| 193 | 4-(4-(5-Chloro-2-methylphenyl)piperazin-1-yl)- | 46-243 | N/A | 522.2 |
| | 2-methyl-1-(4-phenyl-6,7-dihydrothieno[3,2-c]- | | | |
| | pyridin-5(4H)-yl)butane-1,4-dione | | | |
| 194 | N-(Furan-2-ylmethyl)-N-methyl-4-oxo-4-(4- | 46-243 | N/A | 409.2 |
| | phenyl-6,7-dihydrothieno[3,2-c]pyridin-5(4H)- | | | |
| | yl)butanamide | | | |
| 195 | 1-((4aR,8aS)-Octahydroisoquinolin-2(1H)-yl)- | 46-243 | N/A | 437.2 |
| | 4-(4-phenyl-6,7-dihydrothieno[3,2-c]pyridin- | | | |
| | 5(4H)-yl)butane-1,4-dione | | | |
| 196 | 1-(4-Phenyl-6,7-dihydrothieno[3,2-c]pyridin- | 46-243 | N/A | 401.1 |
| | 5(4H)-yl)-4-thiomorpholinobutane-1,4-dione | | | |
| 197 | 1-(4-Phenyl-6,7-dihydrothieno[3,2-c]pyridin- | 46-243 | N/A | 446.2 |
| | 5(4H)-yl)-4-(2-(pyridin-3-yl)pyrrolidin-1- | | | |
| | yl)butane-1,4-dione | | | |
| 198 | N-Benzyl-4-(4-(4-fluorophenyl)-6,7- | 46-243 | N/A | 465.2 |
| | dihydrothieno[3,2-c]pyridin-5(4H)-yl)-N- | | | |
| | isopropyl-4-oxobutanamide | | | |
| 199 | N-Benzyl-4-(4-(4-fluorophenyl)-6,7- | 46-243 | N/A | 437.2 |
| | dihydrothieno[3,2-c]pyridin-5(4H)-yl)-N-methyl- | | | |
| | 4-oxobutanamide | | | |
| 200 | N-(3,4-Dimethoxyphenethyl)-4-(4-(4- | 46-243 | N/A | 511.2 |
| | fluorophenyl)-6,7-dihydrothieno[3,2-c]pyridin- | | | |
| | 5(4H)-yl)-N-methyl-4-oxobutanamide | | | |
| 201 | 1-(4-(4-Fluorophenyl)-6,7-dihydrothieno[3,2-c]- | 46-243 | N/A | 492.2 |
| | pyridin-5(4H)-yl)-4-(4-methyl-2- | | | |
| | phenylpiperazin-1-yl)butane-1,4-dione | | | |
| 202 | 1-(4-(4-Chlorophenyl)-6,7-dihydrothieno[3,2-c]- | 46-243 | N/A | 493.2 |
| | pyridin-5(4H)-yl)-4-(4-phenylpiperidin-1- | | | |
| | yl)butane-1,4-dione | | | |
| 203 | 4-(4-(4-Chlorophenyl)-6,7-dihydrothieno[3,2-c]- | 46-243 | N/A | 454.1 |
| | pyridin-5(4H)-yl)-4-oxo-N-(2-(pyridin-2- | | | |
| | yl)ethyl)butanamide | | | |
| 204 | N-Benzyl-2-methyl-4-oxo-N-phenethyl-4-(4- | 46-243 | N/A | 523.2 |
| | phenyl-6,7-dihydrothieno[3,2-c]pyridin-5(4H)- | | | |
| | yl)butanamide | | | |
| 205 | 1-(4-(4-Fluorophenyl)-6,7-dihydrothieno[3,2-c]- | 46-243 | N/A | 506.2 |
| | pyridin-5(4H)-yl)-4-(2-((6-methylpyridin-2- | | | |
| | yl)methyl)piperidin-1-yl)butane-1,4-dione | | | |
| 206 | 1-(4-(4-Fluorophenyl)-6,7-dihydrothieno- | 46-243 | N/A | 506.2 |
| | 3,2-c]pyridin-5(4H)-yl)-4-(4-(3-methylbenzyl)- | | | |
| | piperazin-1-yl)butane-1,4-dione | | | |
| 207 | 1-(4-(4-Chlorophenyl)-6,7-dihydrothieno- | 46-243 | N/A | 508.2 |
| | [3,2-c]pyridin-5(4H)-yl)-4-(2-(pyridin-4- | | | |
| | ylmethyl)piperidin-1-yl)butane-1,4-dione | | | |
| 208 | 1-(4-(4-Chlorophenyl)-6,7-dihydrothieno- | 46-243 | N/A | 536.2 |
| | [3,2-c]pyridin-5(4H)-yl)-4-(2-((5-ethylpyridin-2- | | | |
| | yl)methyl)piperidin-1-yl)butane-1,4-dione | | | |
| 209 | 1-(4-(4-Chlorophenyl)-6,7-dihydrothieno- | 46-243 | N/A | 522.2 |
| | [3,2-c]pyridin-5(4H)-yl)-4-(2-((3-methylpyridin- | | | |
| | 2-yl)methyl)piperidin-1-yl)butane-1,4-dione | | | |
| 210 | 1-(4-(4-Chlorophenyl)-6,7-dihydrothieno- | 46-243 | N/A | 528.1 |
| | [3,2-c]pyridin-5(4H)-yl)-4-(4-(3-chlorophenyl)- | | | |
| | piperazin-1-yl)butane-1,4-dione | | | |
| 211 | 1-(4-(4-Chlorophenyl)-6,7-dihydrothieno[3,2-c]- | 46-243 | N/A | 522.2 |
| | pyridin-5(4H)-yl)-4-(4-(2,3-dimethylphenyl)- | | | |
| | piperazin-1-yl)butane-1,4-dione | | | |
| 212 | 1-(4-(4-Chlorophenyl)-6,7-dihydrothieno- | 46-243 | N/A | 522.2 |
| | [3,2-c]pyridin-5(4H)-yl)-4-(4-(3,4-dimethyl- | | | |
| | phenyl)piperazin-1-yl)butane-1,4-dione | | | |
| 213 | 1-(4-(4-Chlorophenyl)-6,7-dihydrothieno- | 46-243 | N/A | 562.1 |
| | [3,2-c]pyridin-5(4H)-yl)-4-(4-(3,4-dichloro- | | | |
| | phenyl)piperazin-1-yl)butane-1,4-dione | | | |
| 214 | 1-(4-(4-tert-Butylbenzyl)piperazin-1-yl)-4-(4-(4- | 46-243 | N/A | 564.2 |
| | chlorophenyl)-6,7-dihydrothieno[3,2-c]pyridin- | | | |
| | 5(4H)-yl)butane-1,4-dione | | | |
| 215 | 1-[4-(3,5-Dichloro-4-pyridyl)-1-piperazinyl]-2- | 46-243 | N/A | 543.1 |
| | methyl-4-(4-phenyl-6,7-dihydro-4H-thieno- | | | |
| | [3,2-c]pyridin-5-yl)butane-1,4-dione | | | |
| 216 | 1-[4-[(4-Methoxyphenyl)methyl]-1-piperazinyl]- | 46-243 | N/A | 518.2 |
| | 4-[4-(p-tolyl)-6,7-dihydro-4H-thieno[3,2-c]- | | | |
| | pyridin-5-yl]butane-1,4-dione | | | |
| 217 | 1-[4-[(2-Fluorophenyl)methyl]-1-piperazinyl]-4- | 46-243 | N/A | 506.2 |
| | [4-(p-tolyl)-6,7-dihydro-4H-thieno[3,2-c]pyridin- | | | |
| | 5-yl]butane-1,4-dione | | | |
| 218 | 1-(4-Methyl-2-phenyl-1-piperazinyl)-4-[4-(p- | 46-243 | N/A | 488.2 |
| | tolyl)-6,7-dihydro-4H-thieno[3,2-c]pyridin-5- | | | |
| | yl]butane-1,4-dione | | | |
| 219 | 1-(4-Phenyl-1-piperidinyl)-4-[4-(p-tolyl)-6,7- | 46-243 | N/A | 473.2 |
| | dihydro-4H-thieno[3,2-c]pyridin-5-yl]butane- | | | |
| | 1,4-dione | | | |
| 220 | 1-[4-(p-Tolyl)-6,7-dihydro-4H-thieno[3,2-c]- | 46-243 | N/A | 460.2 |
| | pyridin-5-yl]-4-[2-(2-pyridyl)-1-pyrrolidinyl]- | | | |
| | butane-1,4-dione | | | |
| 221 | 1-[4-(p-Tolyl)-6,7-dihydro-4H-thieno[3,2-c]- | 46-243 | N/A | 460.2 |
| | pyridin-5-yl]-4-[3-(3-pyridyl)-1-pyrrolidinyl]- | | | |
| | butane-1,4-dione | | | |
| 222 | 4-Oxo-4-[4-(p-tolyl)-6,7-dihydro-4H-thieno- | 46-243 | N/A | 434.2 |
| | [3,2-c]pyridin-5-yl]-N-[2-(2-pyridyl)ethyl]- | | | |
| | butanamide | | | |
| 223 | 1-[4-[(4-Methoxyphenyl)methyl]-1-piperazinyl]- | 46-243 | N/A | 518.2 |
| | 4-[4-(m-tolyl)-6,7-dihydro-4H-thieno[3,2-c]- | | | |
| | pyridin-5-yl]butane-1,4-dione | | | |
| 224 | 1-[4-[(2-Fluorophenyl)methyl]-1-piperazinyl]-4- | 46-243 | N/A | 506.2 |
| | [4-(m-tolyl)-6,7-dihydro-4H-thieno[3,2-c]pyridin- | | | |
| | 5-yl]butane-1,4-dione | | | |
| 225 | 1-(4-Methyl-2-phenyl-1-piperazinyl)-4-[4-(m- | 46-243 | N/A | 488.2 |
| | tolyl)-6,7-dihydro-4H-thieno[3,2-c]pyridin-5- | | | |
| | yl]butane-1,4-dione | | | |
| 226 | 1-[4-(m-Tolyl)-6,7-dihydro-4H-thieno[3,2-c]- | 46-243 | N/A | 473.2 |
| | pyridin-5-yl]-4-(4-phenyl-1-piperidinyl)butane- | | | |
| | 1,4-dione | | | |
| 227 | 1-[4-(m-Tolyl)-6,7-dihydro-4H-thieno[3,2-c]- | 46-243 | N/A | 460.2 |
| | pyridin-5-yl]-4-[2-(2-pyridyl)-1-pyrrolidinyl]- | | | |
| | butane-1,4-dione | | | |
| 228 | 1-[4-(m-Tolyl)-6,7-dihydro-4H-thieno[3,2-c]- | 46-243 | N/A | 460.2 |
| | pyridin-5-yl]-4-[3-(3-pyridyl)-1-pyrrolidinyl]- | | | |
| | butane-1,4-dione | | | |
| 229 | N-Methyl-4-[4-(m-tolyl)-6,7-dihydro-4H- | 46-243 | N/A | 448.2 |
| | thieno[3,2-c]pyridin-5-yl]-4-oxo-N-[2-(4- | | | |
| | pyridyl)ethyl]butanamide | | | |
| 230 | 1-[4-(3-Fluorophenyl)-6,7-dihydro-4H- | 46-243 | N/A | 522.2 |
| | thieno[3,2-c]pyridin-5-yl]-4-[4-[(4-methoxy- | | | |
| | phenyl)methyl]-1-piperazinyl]butane-1,4-dione | | | |
| 231 | 1-[4-(3-Fluorophenyl)-6,7-dihydro-4H- | 46-243 | N/A | 510.2 |
| | thieno[3,2-c]pyridin-5-yl]-4-[4-[(2-fluoro- | | | |
| | phenyl)methyl]-1-piperazinyl]butane-1,4-dione | | | |
| 232 | 4-[4-(m-Tolyl)-6,7-dihydro-4H-thieno[3,2-c]- | 46-243 | N/A | 419.2 |
| | pyridin-5-yl]-4-oxo-N-(p-tolyl)butanamide | | | |
| 233 | N-(2,4-Dimethylphenyl)-4-[4-(m-tolyl)-6,7- | 46-243 | N/A | 433.2 |
| | dihydro-4H-thieno[3,2-c]pyridin-5-yl]-4- | | | |
| | oxobutanamide | | | |
| 234 | 4-[4-(3-Fluorophenyl)-6,7-dihydro-4H- | 46-243 | N/A | 463.2 |
| | thieno[3,2-c]pyridin-5-yl]-N-(1-methyl-6- | | | |
| | indazolyl)-4-oxobutanamide | | | |
| 235 | 4-[4-(3-Fluorophenyl)-6,7-dihydro-4H- | 46-243 | N/A | 423.1 |
| | thieno[3,2-c]pyridin-5-yl]-4-oxo-N-(p- | | | |
| | tolyl)butanamide | | | |
| 236 | 4-[4-(3-Fluorophenyl)-6,7-dihydro-4H- | 46-243 | N/A | 409.1 |
| | thieno[3,2-c]pyridin-5-yl]-4-oxo-N- | | | |
| | phenylbutanamide | | | |
| 237 | N-(2-Chlorophenyl)-4-[4-(3-fluorophenyl)-6,7- | 46-243 | N/A | 443.1 |
| | dihydro-4H-thieno[3,2-c]pyridin-5-yl]-4- | | | |
| | oxobutanamide | | | |
| 238 | N-(2,4-Dimethylphenyl)-4-[4-(3-fluorophenyl)- | 46-243 | N/A | 437.2 |
| | 6,7-dihydro-4H-thieno[3,2-c]pyridin-5-yl]-4- | | | |
| | oxobutanamide | | | |
| 239 | 4-[4-(4-Chlorophenyl)-6,7-dihydro-4H- | 46-243 | N/A | 479.1 |
| | thieno[3,2-c]pyridin-5-yl]-N-(1-methyl-6- | | | |
| | indazolyl)-4-oxobutanamide | | | |
| 240 | 4-[4-(4-Chlorophenyl)-6,7-dihydro-4H- | 46-243 | N/A | 439.1 |
| | thieno[3,2-c]pyridin-5-yl]-4-oxo-N-(p- | | | |
| | tolyl)butanamide | | | |
| 241 | 4-[4-(4-Chlorophenyl)-6,7-dihydro-4H- | 46-243 | N/A | 455.1 |
| | thieno[3,2-c]pyridin-5-yl]-N-(4-methoxyphenyl)- | | | |
| | 4-oxobutanamide | | | |
| 242 | 4-[4-(4-Chlorophenyl)-6,7-dihydro-4H- | 46-243 | N/A | 425.1 |
| | thieno[3,2-c]pyridin-5-yl]-4-oxo-N- | | | |
| | phenylbutanamide | | | |
| 243 | 4-[4-(4-Chlorophenyl)-6,7-dihydro-4H- | 46-243 | N/A | 453.1 |
| | thieno[3,2-c]pyridin-5-yl]-N-(2,4- | | | |
| | dimethylphenyl)-4-oxobutanamide | | | |
| 244 | 4-[4-(2,6-Dimethylphenyl)-6,7-dihydro-4H- | 9 | 33 | 501.2 |
| | thieno[3,2-c]pyridin-5-yl]-4-oxo-N-[[3- | | | |
| | (trifluoromethyl)phenyl]methyl]butanamide | | | |
| 245 | N-(2-Cyclohexylethyl)-4-oxo-4-(4-phenyl-6,7- | 34 | 66 | 425.2 |
| | dihydro-4H-thieno[3,2-c]pyridin-5- | | | |
| | yl)butanamide | | | |
| 246 | N-(3,3-Dimethylbutyl)-4-oxo-4-(4-phenyl-6,7- | 34 | 50 | 399.2 |
| | dihydro-4H-thieno[3,2-c]pyridin-5- | | | |
| | yl)butanamide | | | |
| 247 | N-(Cyclohexylmethyl)-4-oxo-4-(4-phenyl-6,7- | 34 | 63 | 411.2 |
| | dihydro-4H-thieno[3,2-c]pyridin-5- | | | |
| | yl)butanamide | | | |
| 248 | N-[[3-Methyl-5-(trifluoromethoxy)phenyl]- | 11 | 71 | 487.2 |
| | methyl]-4-oxo-4-(4-phenyl-6,7-dihydro-4H- | | | |
| | thieno[3,2-c]pyridin-5-yl)butanamide | | | |
| 249 | N-[[4-Methyl-3-(trifluoromethyl)phenyl]methyl]- | 11 | 51 | 487.2 |
| | 4-oxo-4-(4-phenyl-6,7-dihydro-4H-thieno- | | | |
| | [3,2-c]pyridin-5-yl)butanamide | | | |
| 250 | N-[[4-Fluoro-3-(trifluoromethyl)phenyl]methyl]- | 11 | 65 | 491.1 |
| | 4-oxo-4-(4-phenyl-6,7-dihydro-4H-thieno- | | | |
| | [3,2-c]pyridin-5-yl)butanamide | | | |
| 251 | 4-[4-(2-Ethylphenyl)-6,7-dihydro-4H-thieno[3,2- | 2 | 68 | 501.2 |
| | c]pyridin-5-yl]-4-oxo-N-[[3- | | | |
| | (trifluoromethyl)phenyl]methyl]butanamide | | | |
| 252 | 4-[4-(2-Isopropylphenyl)-6,7-dihydro-4H- | 9 | 35 | 515.2 |
| | thieno[3,2-c]pyridin-5-yl]-4-oxo-N-[[3- | | | |
| | (trifluoromethyl)phenyl]methyl]butanamide | | | |
| 253 | N-(3-Cyclohexylpropyl)-4-oxo-4-(4-phenyl-6,7- | 34 | 77 | 439.2 |
| | dihydro-4H-thieno[3,2-c]pyridin-5- | | | |
| | yl)butanamide | | | |
| 254 | 4-[4-(3-Methyl-2-thienyl)-6,7-dihydro-4H- | 2 | 39 | 493.1 |
| | thieno[3,2-c]pyridin-5-yl]-4-oxo-N-[[3- | | | |
| | (trifluoromethyl)phenyl]methyl]butanamide | | | |
| 255 | (1S,2S)-2-[Oxo-(4-phenyl-6,7-dihydro-4H- | 2 | 89 | 485.1 |
| | thieno[3,2-c]pyridin-5-yl)methyl]-N-[[3-(trifluoro- | | | |
| | methyl)phenyl]methyl]-1-cyclopropane- | | | |
| | carboxamide | | | |
| 256 | 4-[4-(o-Tolyl)-6,7-dihydro-4H-thieno[3,2-c]- | 34 | 61 | 487.2 |
| | pyridin-5-yl]-4-oxo-N-[[2-(trifluoromethyl)- | | | |
| | phenyl]methyl]butanamide | | | |
| 257 | 4-[4-(o-Tolyl)-6,7-dihydro-4H-thieno[3,2-c]- | 34 | 51 | 487.2 |
| | pyridin-5-yl]-4-oxo-N-[[4-(trifluoromethyl)- | | | |
| | phenyl]methyl]butanamide | | | |
| 258 | 4-(7-Butyl-5,7-dihydro-4H-thieno[2,3-c]pyridin- | 2 | | 453.2 |
| | 6-yl)-4-oxo-N-[[3-(trifluoromethyl)phenyl]- | | | |
| | methyl]butanamide | | | |
| 259 | 4-[4-(Cyclohexylmethyl)-6,7-dihydro-4H- | 2 | | 493.2 |
| | thieno[3,2-c]pyridin-5-yl]-4-oxo-N-[[3-(trifluoro- | | | |
| | methyl)phenyl]methyl]butanamide | | | |
| 260 | 4-(4-Cyclopropyl-6,7-dihydro-4H-thieno[3,2-c]- | 2 | | 437.1 |
| | pyridin-5-yl)-4-oxo-N-[[3-(trifluoromethyl)- | | | |
| | phenyl]methyl]butanamide | | | |
| 261 | 4-Oxo-4-(4-phenthyl-6,7-dihydro-4H-thieno- | 2 | | 501.2 |
| | [3,2-c]pyridin-5-yl)-N-[[3-(trifluoromethyl)- | | | |
| | phenyl]methyl]butanamide | | | |
| 262 | 4-[4-(4-Fluoro-2-methylphenyl)-6,7-dihydro-4H- | 2 | | 505.1 |
| | thieno[3,2-c]pyridin-5-yl]-4-oxo-N-[[3- | | | |
| | (trifluoromethyl)phenyl]methyl]butanamide | | | |
| 263 | 4-(4-Cyclopentyl-6,7-dihydro-4H-thieno[3,2-c]- | 2 | | 465.2 |
| | pyridin-5-yl)-4-oxo-N-[[3-(trifluoromethyl)- | | | |
| | phenyl]methyl]butanamide | | | |
| 264 | N-[[2-Methyl-3-(trifluoromethyl)phenyl]methyl]- | 44 | | 487.2 |
| | 4-oxo-4-(4-phenyl-6,7-dihydro-4H-thieno- | | | |
| | [3,2-c]pyridin-5-yl)butanamide | | | |
| 265 | 4-Oxo-4-[4-(3-phenylpropyl)-6,7-dihydro-4H- | 2 | | 515.2 |
| | thieno[3,2-c]pyridin-5-yl]-N-[[3-(trifluoromethyl)- | | | |
| | phenyl]methyl]butanamide | | | |
| 266 | N-[[2-Methyl-5-(trifluoromethyl)phenyl]methyl]- | 44 | | 487.2 |
| | 4-oxo-4-(4-phenyl-6,7-dihydro-4H-thieno- | | | |
| | [3,2-c]pyridin-5-yl)butanamide | | | |
| 267 | N-Cyclohexyl-4-oxo-4-(4-phenyl-6,7-dihydro- | 44 | | 397.2 |
| | 4H-thieno[3,2-c]pyridin-5-yl)butanamide | | | |
| 268 | N-(2,2-Dimethylpropyl)-4-oxo-4-(4-phenyl-6,7- | 44 | | 385.2 |
| | dihydro-4H-thieno[3,2-c]pyridin-5- | | | |
| | yl)butanamide | | | |
| 269 | 1-(4-Phenyl-6,7-dihydro-4H-thieno[3,2-c]- | 24 | | 499.2 |
| | pyridin-5-yl)-4-[7-(trifluoromethyl)-3,4-dihydro- | | | |
| | 1H-isoquinolin-2-yl]butane-1,4-dione | | | |
| 270 | 4-Oxo-N-pentyl-4-(4-phenyl-6,7-dihydro-4H- | 44 | | 385.2 |
| | thieno[3,2-c]pyridin-5-yl)butanamide | | | |
| 271 | 1-(4-Phenyl-6,7-dihydro-4H-thieno[3,2-c]- | 24 | | 499.2 |
| | pyridin-5-yl)-4-[5-(trifluoromethyl)-3,4-dihydro- | | | |
| | 1H-isoquinolin-2-yl]butane-1,4-dione | | | |
| 272 | 4-Oxo-4-(4-phenyl-6,7-dihydro-4H-thieno- | 44 | | 513.2 |
| | [3,2-c]pyridin-5-yl)-N-[5-(trifluoromethyl)-1- | | | |
| | tetralinyl]butanamide | | | |
| 273 | 4-Oxo-4-(4-phenyl-6,7-dihydro-4H-thieno- | 44 | | 513.2 |
| | [3,2-c]pyridin-5-yl)-N-[7-(trifluoromethyl)-1- | | | |
| | tetralinyl]butanamide | | | |
| 274 | 4-[7-(o-Tolyl)-5,7-dihydro-4H-thieno[2,3-c]- | 2 | | 487.2 |
| | pyridin-6-yl]-4-oxo-N-[[3-(trifluoromethyl)- | | | |
| | phenyl]methyl]butanamide | | | |
| 275 | 4-(7-Cyclohexyl-5,7-dihydro-4H-thieno[2,3-c]- | 2 | | 479.2 |
| | pyridin-6-yl)-4-oxo-N-[[3-(trifluoromethyl)- | | | |
| | phenyl]methyl]butanamide | | | |

Pharmacological Experiments

Fluorescence Assay using a Voltage Sensitive Dye

Human CHO-K1 cells expressing KCNQ2/3 channels are cultivated adherently at 37° C., 5% CO₂ and 95% humidity in cell culture bottles (e.g. 80 cm² TC flasks, Nunc) with DMEM-high glucose (Sigma Aldrich, D7777) including 10% FCS (PAN Biotech, e.g. 3302-P270521) or alternatively MEM Alpha Medium (1×, liquid, Invitrogen, #22571), 10% fetal calf serum (FCS) (Invitrogen, #10270-106, heat-inactivated) and the necessary selection antibiotics.

Before being sown out for the measurements, the cells are washed with a 1×DPBS buffer without Ca²⁺/Mg²⁺ (e.g. Invitrogen, #14190-094) and detached from the bottom of the culture vessel by means of Accutase (PAA Laboratories, #L11-007) (incubation with Accutase for 15 min at 37° C.). The cell count then present is determined using a CASY™ cell counter (TCC model, Schärfe System) in order subsequently to apply, depending on the density optimization for the individual cell line, 20,000-30,000 cells/well/100 μl of the described nutrient medium to 96-well measuring plates of the Corning™ CellBIND™ type (Flat Clear Bottom Black Polystyrene Microplates, #3340). Incubation is then carried out for one hour at room temperature, without gassing or adjusting the humidity, followed by incubation for 24 hours at 37° C., 5% CO₂ and 95% humidity.

The voltage-sensitive fluorescent dye from the Membrane Potential Assay Kit (Red™ Bulk format part R8123 for FLIPR, MDS Analytical Technologies™) is prepared by dissolving the contents of a vessel Membrane Potential Assay Kit Red Component A in 200 ml of extracellular buffer (ES buffer, 120 mM NaCl, 1 mM KCl, 10 mM HEPES, 2 mM CaCl₂, 2 mM MgCl₂, 10 mM glucose; pH 7.4). After removal of the nutrient medium, the cells are washed with 200 μl of ES buffer, then covered with a layer of 100 μl of the dye solution prepared above and incubated for 45 min at room temperature with the exclusion of light.

The fluorescence measurements are carried out with a BMG Labtech FLUOstar™, BMG Labtech NOVOstar™ or BMG Labtech POLARstar™ instrument (525 nm excitation, 560 nm emission, Bottom Read mode). After incubation of the dye, 50 μl of the test substances in the desired concentrations, or 50 μl of ES buffer for control purposes, are introduced into separate cavities of the measuring plate and incubated for 30 min at room temperature while being shielded from light. The fluorescence intensity of the dye is then measured for 5 min and the fluorescence value F₁ of each well is thus determined at a given, constant time. 15 μl of a 100 mM KCl solution (final concentration 92 mM) are then added to each well. The change in fluorescence is subsequently measured until all the relevant measured values have been obtained (mainly 5-30 min). At a given time after KCl application, a fluorescence value F₂ is determined, in this case at the time of the fluorescence peak.

For calculation, the fluorescence intensity F₂ is compared with the fluorescence intensity F₁, and the agonistic activity of the target compound on the potassium channel is determined therefrom. F₂ and F₁ are calculated as follows:

$$( \frac{{F}_{2} - {F}_{1}}{{F}_{1}} ) \times 100 = \frac{Δ F}{F} ( \% )$$

In order to determine whether a substance has agonistic activity,

$$\frac{Δ F}{F} ,$$

for example, can be compared with

$${( \frac{Δ F}{F} )}_{K}$$

of control cells.

$${( \frac{Δ F}{F} )}_{K}$$

is determined by adding to the reaction batch only the buffer solution instead of the test substance, determining the value F_{1K} of the fluorescence intensity, adding the potassium ions as described above, and measuring a value F_{2K} of the fluorescence intensity. F_{2K} and F_{1K} are then calculated as follows:

$$( \frac{{F}_{2 K} - {F}_{1 K}}{{F}_{1 K}} ) \times 100 = {( \frac{Δ F}{F} )}_{K} ( \% )$$

A substance has an agonistic activity on the potassium channel when

$$\frac{Δ F}{F}$$

is greater than

$${( \frac{Δ F}{F} )}_{k} \text{:}$$

$$\frac{Δ F}{F} 〉 {( \frac{Δ F}{F} )}_{K}$$

Independently of the comparison of

$$\frac{Δ F}{F} with {( \frac{Δ F}{F} )}_{K}$$

it is possible to conclude that a target compound has agonistic activity if an increase in

$$\frac{Δ F}{F}$$

is to be observed as the dosage of the target compound increases.

Calculations of EC₅₀ and IC₅₀ values are carried out with the aid of ‘Prism v4.0’ software (GraphPad Software™).

Voltage Clamp Measurements

In order to confirm a KCNQ2/3-agonistic action of the substances electro-physiologically, patch-clamp measurements (Hamill O P, Marty A, Neher E, Sakmann B, Sigworth F J.: Improved patch-clamp techniques for high-resolution current recording from cells and cell-free membrane patches, Pflugers Arch. 1981 August; 391(2):85-100) were carried out in voltage clamp mode on a stably transfected hKCNQ2/3 CHO-K1 cell line. After formation of the gigaseal, the cells were first clamped at a holding potential of −60 mV. Thereafter, depolarizing voltage jumps were applied up to a potential of +20 mV (increment: 20 mV, duration: 1 second) in order to confirm the functional expression of KCNQ2/3-typical currents. The testing of the substances was carried out at a potential of −40 mV. The increase in current induced by retigabine (10 μM) at −40 mV was first recorded as a positive control on each cell. After complete washing out of the retigabine effect (duration: 80 s), the test substance (10 μM) was applied. The increase in current induced by the test substance was standardized to the retigabine effect and indicated as the relative efficacy (see below).

Formalin Test in the Rat

The formalin test (Dubuisson, D. and Dennis, S. G., 1977, Pain, 4, 161-174) represents a model for both acute and chronic pain. In the studies presented here, the chronic pain component (phase II of the formalin test; time period 21-27 min after formalin administration) was evaluated. A biphase nociceptive reaction is induced in freely mobile test animals by a single formalin injection into the dorsal side of a rear paw, the reaction being detected by observation of three clearly distinguishable behavior patterns. Formalin is administered subcutaneously into the dorsal side of the right rear paw of each animal in a volume of 50 μl and a concentration of 5%. The vehicle and the test substances are administered intravenously 5 min, or orally 30 min, before the formalin injection. The specific changes in behavior, such as lifting and shaking of the paw, weight displacement of the animal and biting and licking reactions, are observed and recorded continuously up to 60 min after the formalin administration. The changes in behavior are given different weightings (score 0-3) and a pain rate (PR) is calculated using the following formula:

PR=[(T₀×0)+(T₁×1)+(T₂×2)+(T₃×3)/180,

where T₀, T₁, T₂, T₃ correspond to the time, in seconds, at which the animal exhibited behavior 0, 1, 2 or 3.

Rats of the strain Sprague Dawley (Janvier, Belgium) are used as the animal strain. The weight of the animals is 180-200 g; the size of the group was n=10.

### Pharmacological Data

The results from the pharmacological models described above are summarized in the following Table 3.

TABLE 3

| | | | | | Formalin test rat |
|---|---|---|---|---|---|
| | Fluorimetry | | | patch-clamp | i.v. @ 10 mg/kg |
| | % Efficacy | | | % Efficacy | % reduction in |
| | @ 10 μM | Fluorimetry | Fluorimetry | @ 10 μM | nociceptive |
| Illustrative | (reti-gabine = | EC₅₀ | IC₅₀ | (retigabine = | behavior |
| compound | 100%) | [nM] | [nM] | 100%) | [% (dose mg/kg)] |
| 1 | 168 | 1690 | | 97 | |
| 2 | 165 | 758 | | 106 | 56 (10.0) |
| 3 | 103 | 357 | | | |
| 4 | 102 | 456 | | | |
| 5 | 61 | >1662 | | | |
| 6 | 133 | 284 | | | |
| 7 | 132 | 348 | | | |
| 8 | 148 | 79 | | | 11 (4.64) |
| 9 | 110 | 407 | | | |
| 10 | 121 | 382 | | | |
| 11 | −18 | | | | |
| 12 | −46 | | | | |
| 13 | 51 | 375 | | | |
| 14 | −54 | | | | |
| 15 | −46 | | | | |
| 16 | 21 | | | | |
| 17 | 74 | 62 | | | 15 (10.0) |
| 18 | 11 | 2181 | | | |
| 19 | −45 | | | | |
| 20 | 54 | 722 | | | |
| 21 | 148 | 324 | | | |
| 22 | 139 | 1219 | | | |
| 23 | 146 | 947 | | | |
| 24 | −56 | | | | |
| 25 | 12 | | | | |
| 26 | −20 | | | | |
| 27 | 119 | >404 | | | |
| 28 | 106 | 540 | | | |
| 29 | −45 | | 133 | | |
| 30 | −101 | | 212 | | |
| 31 | −44 | | 250 | | |
| 32 | 110 | 414 | | | |
| 33 | −6 | | | | |
| 34 | 179 | 628 | | | |
| 35 | | 572 | | | |
| 36 | | 707 | | | |
| 40 | | 156 | | | |
| 41 | | 261 | | | |
| 44 | −21 | | | | |
| 45 | 39 | 311 | | | |
| 46 | 35 | | | | |
| 47 | 37 | | | | |
| 48 | −15 | | | | |
| 49 | 38 | | | | |
| 50 | 10 | | | | |
| 51 | 38 | | | | |
| 52 | 15 | | | | |
| 53 | 28 | | | | |
| 54 | 19 | | | | |
| 245 | | 690 | | | |
| 247 | | 1438 | | | |
| 249 | | 347 | | | |
| 250 | | 517 | | | |
| 251 | | 131 | | | |
| 252 | | 1012 | | | |
| 254 | | 211 | | | |
| 256 | | 90 | | | |
| 257 | | 547 | | | |
| 258 | | 306 | | | |
| 260 | | 108 | | 91 | 46 (4.64) |
| 261 | | 84 | | | |
| 262 | | 166 | | | |
| 263 | | 160 | | | |
| 265 | | 596 | | | |
| 269 | | 519 | | | |
| 271 | | 431 | | | |
| 274 | | 323 | | | |
| 275 | | 962 | | | |

Comparison Experiments

The substituted tetrahydrothienopyridines according to the invention are distinguished, as compared with substituted tetrahydropyrrolopyrazines known from WO 2008/046582, by improved activity in vitro and in vivo, as is illustrated by the following comparison experiments:

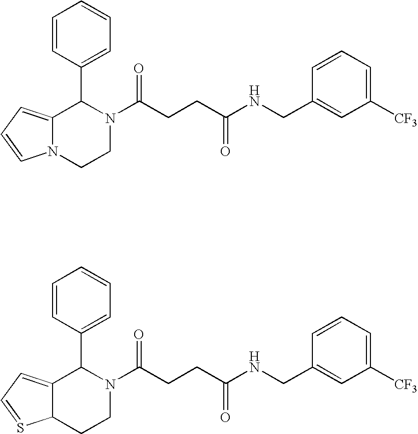

Example No. 2 of WO2008/046582 compound 2 of the invention

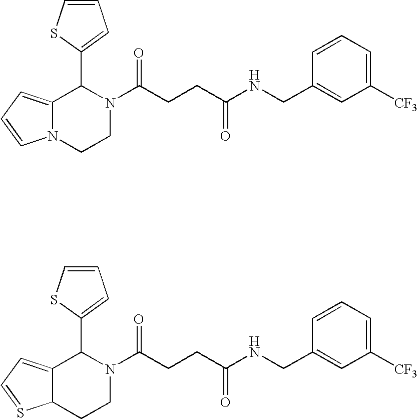

Example No. 76 of WO2008/046582 compound 1 of the invention

| | | | Formalin test rat |
|---|---|---|---|
| | | | i.v. @ 10 mg/kg |
| | Fluorimetry % | Fluorimetry | % reduction in |
| | Efficacy @ 10 μM | EC₅₀ | noiceptive |
| | (retigabine = 100%) | [μM] | behavior [%] |
| Example No. 2 of | 115 | 2.81 | no effect |
| WO 2008/046582 | | | |
| 2 | 145 | 1.44 | 56 |
| Example No. 76 | 113 | 3.29 | not tested |
| of WO | | | |
| 2008/046582 | | | |
| 1 | 165 | 0.76 | not tested |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A substituted tetrahydrothienopyridine compound corresponding to formula (1): 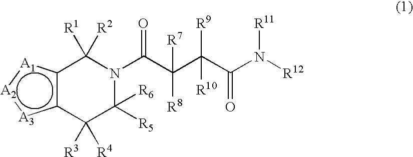
wherein
A₁ represents S, A₂ represents CR¹⁴ and A₃ represents CR¹⁵; or
A₁ represents CR¹³, A₂ represents S and A₃ represents CR¹⁵; or
A₁ represents CR¹³, A₂ represents CR¹⁴ and A₃ represents S;
R⁰ represents:
saturated or unsaturated, branched or unbranched, unsubstituted or mono- or poly-substituted C₁₋₈-alkyl;
saturated or unsaturated, unsubstituted or mono- or poly-substituted C₃₋₇-cycloalkyl or heterocyclyl;
unsubstituted or mono- or poly-substituted aryl or heteroaryl;
saturated or unsaturated, unsubstituted or mono- or poly-substituted C₁₋₈-alkyl-bridged C₃₋₇-cycloalkyl or heterocyclyl, wherein the alkyl chain optionally may be branched or unbranched, saturated or unsaturated, unsubstituted, mono- or poly-substituted; or
unsubstituted or mono- or poly-substituted C₁₋₈-alkyl-bridged aryl or heteroaryl, wherein the alkyl chain optionally may be branched or unbranched, saturated or unsaturated, unsubstituted, mono- or poly-substituted;
R¹ represents H; F; Cl; Br; CN; or R⁰;
R² represents H; F; Cl; Br; or saturated or unsaturated, branched or unbranched, unsubstituted or mono- or poly-substituted C₁₋₈-alkyl; or
R¹ and R² together with the carbon atom to which they are attached form a saturated or unsaturated, unsubstituted or mono- or poly-substituted C₃₋₇-cycloalkyl or heterocyclyl ring optionally fused with unsubstituted or mono- or poly-substituted aryl or heteroaryl;
R³, R⁴, R⁵, R⁶ each independently represent H; F; Cl; Br; saturated or unsaturated, branched or unbranched, unsubstituted or mono- or poly-substituted C₁₋₈-alkyl; or unsubstituted or mono- or poly-substituted phenyl;
R⁷ represents H; F; Cl; Br; CN; R⁰; OR⁰; O—C(═O)—R⁰; O—C(═O)—O—R⁰; O—(C═O)—NH—R⁰; O—C(═O)—N(R⁰)₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; C(═O)—OH; C(═O)—OR⁰; C(═O)—NH₂; C(═O)—NH—R⁰; C(═O)—N(R⁰)₂; S(═O)₂—OH; S(═O)₂OR⁰; S(═O)₂—NH₂; S(═O)₂—NH—R⁰; or S(═O)₂—N(R⁰)₂;
R⁸ represents H; F; Cl; Br; CN; or saturated or unsaturated, branched or unbranched, unsubstituted or mono- or poly-substituted C₁₋₈-alkyl;
R⁹ represents H; F; Cl; Br; CN; saturated or unsaturated, branched or unbranched, unsubstituted or mono- or poly-substituted C₁₋₈-alkyl; saturated or unsaturated, unsubstituted or mono- or poly-substituted C₃₋₇-cycloalkyl or heterocyclyl; unsubstituted or mono- or poly-substituted aryl or heteroaryl; saturated or unsaturated, unsubstituted or mono- or poly-substituted C₁₋₈-alkyl-bridged C₃₋₇-cycloalkyl or heterocyclyl, wherein the alkyl chain optionally may be branched or unbranched, saturated or unsaturated, unsubstituted, mono- or poly-substituted; unsubstituted or mono- or poly-substituted C₂₋₈-alkyl-bridged aryl or heteroaryl, wherein the alkyl chain optionally may be branched or unbranched, saturated or unsaturated, unsubstituted, mono- or poly-substituted; saturated or unsaturated, branched or unbranched, unsubstituted or mono- or poly-substituted O—C₁₋₈-alkyl; saturated or unsaturated, unsubstituted or mono- or poly-substituted O—C₃₋₇-cycloalkyl or O-heterocyclyl; saturated or unsaturated, unsubstituted or mono- or poly-substituted O—C₁₋₈-alkyl-bridged C₃₋₇-cycloalkyl or heterocyclyl, wherein the alkyl chain optionally may be branched or unbranched, saturated or unsaturated, unsubstituted, mono- or poly-substituted; unsubstituted or mono- or poly-substituted O—C₁₋₈-alkyl-bridged aryl or heteroaryl, wherein the alkyl chain optionally may be branched or unbranched, saturated or unsaturated, unsubstituted, mono- or poly-substituted; O—C(═O)—R⁰; O—C(═O)—O—R⁰; O—(C═O)—NH—R⁰; O—C(═O)—N(R⁰)₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; C(═O)—OH; C(═O)—OR⁰; C(═O)—NH₂; C(═O)—NH—R⁰; C(═O)—N(R⁰)₂; S(═O)₂—OH; S(═O)₂OR⁰; S(═O)₂—NH₂; or S(═O)₂—NH—R⁰; S(═O)₂—N(R⁰)₂;
R¹⁰ represents H; F; Cl; Br; CN; or saturated or unsaturated, branched or unbranched, unsubstituted or mono- or poly-substituted C₁₋₈-alkyl; or
R⁷ and R⁹ together with the carbon atoms to which they are attached form a saturated or unsaturated, unsubstituted or mono- or poly-substituted C₃₋₇-cycloalkyl or heterocyclyl ring optionally fused with unsubstituted or mono- or poly-substituted aryl or heteroaryl; or
R⁷ and R⁸; or R⁹ and R¹⁰, together with the carbon atoms to which they are attached form a saturated or unsaturated, unsubstituted or mono- or poly-substituted C₃₋₇-cycloalkyl or heterocyclyl ring optionally fused with unsubstituted or mono- or poly-substituted aryl or heteroaryl;
R¹¹ represents H; saturated or unsaturated, branched or unbranched, unsubstituted or mono- or poly-substituted C₁₋₈-alkyl; or saturated or unsaturated, unsubstituted or mono- or poly-substituted C₃₋₇-cycloalkyl;
R¹² represents C₂₋₁₆-alkyl, saturated or unsaturated; branched or unbranched, unsubstituted or mono- or poly-substituted; C₃₋₇-cycloalkyl or heterocyclyl, in each case saturated or unsaturated, unsubstituted or mono- or poly-substituted; aryl or heteroaryl, unsubstituted or mono- or poly-substituted; C₁₋₈-alkyl-bridged C₃₋₇-cycloalkyl or heterocyclyl, in each case saturated or unsaturated, unsubstituted or mono- or poly-substituted, wherein the alkyl chain in each case can be branched or unbranched, saturated or unsaturated, unsubstituted, mono- or poly-substituted; C₁₋₈-alkyl-bridged aryl or heteroaryl, in each case unsubstituted or mono- or poly-substituted; wherein the alkyl chain in each case can be branched or unbranched, saturated or unsaturated, unsubstituted, mono- or poly-substituted; or
R¹¹ and R¹² together with the nitrogen atom to which they are attached form form a saturated or unsaturated, unsubstituted or mono- or poly-substituted heterocyclyl ring optionally fused with unsubstituted or mono- or poly-substituted aryl or heteroaryl; and
R¹³, R¹⁴ and R¹⁵ each independently represent H; F; Cl; Br; I; NO; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; O—(C₁₋₈-alkyl)-O; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)₂R⁰; S(═O)₂OH; S(═O)₂OR⁰; S(═O)₂NH₂; S(═O)₂NHR⁰; or S(═O)₂N(R⁰)₂;
wherein
“substituted alkyl”, “substituted heterocyclyl” and “substituted cycloalkyl” denote the replacement of one or more hydrogen atoms, in each case each independently, by F; Cl; Br; I; CN; CF₃; ═O; ═NH; ═C(NH₂)₂; NO₂; R⁰; C(═O)H; C(═O)R⁰; CO₂H; C(═O)OR⁰; CONH₂; C(═O)NHR⁰; C(═O)N(R⁰)₂; OH; OR⁰; O—(C₁₋₈-alkyl)-O; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂; and
“substituted aryl” and “substituted heteroaryl” denote the replacement of one or more hydrogen atoms, in each case each independently, by F; Cl; Br; I; NO; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; O—(C₁₋₈-alkyl)-O; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
with the exception of the following compounds:
1-morpholino-4-(4-(thiophen-2-yl)-6,7-dihydrothieno[3,2-c]pyridin-5(4H)-yl)-butane-1,4-dione,
1-(4-acetylpiperazin-1-yl)-4-(4-(thiophen-2-yl)-6,7-dihydrothieno[3,2-c]pyridin-5(4H)-yl)butane-1,4-dione and
1-(3-phenyl-4,5-dihydropyrazol-1-yl)-4-(4-(thiophen-2-yl)-6,7-dihydrothieno-[3,2-c]pyridin-5(4H)-yl)butane-1,4-dione;
in the form of a free compound or of a physiologically acceptable salt.

2. A compound as claimed in claim 1, wherein R¹² represents:
saturated or unsaturated; branched or unbranched, unsubstituted or mono- or poly-substituted C₄₋₁₆-alkyl;
saturated or unsaturated, unsubstituted or mono- or poly-substituted C₃₋₇-cycloalkyl or heterocyclyl;
unsubstituted or mono- or poly-substituted aryl or heteroaryl;
saturated or unsaturated, unsubstituted or mono- or poly-substituted C₁₋₈-alkyl-bridged C₃₋₇-cycloalkyl or heterocyclyl, wherein the alkyl chain optionally may be branched or unbranched, saturated or unsaturated, unsubstituted, mono- or poly-substituted; or
unsubstituted or mono- or poly-substituted C₁₋₈-alkyl-bridged aryl or heteroaryl, wherein the alkyl chain optionally may be branched or unbranched, saturated or unsaturated, unsubstituted, mono- or poly-substituted.

3. A compound as claimed in claim 1, wherein
R⁷ represents H; F; Cl; Br; CN; OH; NH₂; saturated or unsaturated, branched or unbranched, unsubstituted or mono- or poly-substituted C₁₋₈-alkyl, O—C₁₋₈-alkyl, NH—C₁₋₈-alkyl, or N(C₁₋₈-alkyl)₂; unsubstituted or mono- or poly-substituted phenyl or heteroaryl; or unsubstituted or mono- or poly-substituted C₁₋₂-alkyl-bridged phenyl or heteroaryl, wherein the alkyl chain optionally may be branched or unbranched, saturated or unsaturated, unsubstituted or mono- or poly-substituted; and
R⁹ represents H; F; Cl; Br; CN; OH; NH₂; saturated or unsaturated, branched or unbranched, unsubstituted or mono- or poly-substituted C₁₋₈-alkyl, O—C₁₋₈-alkyl, NH—C₁₋₈-alkyl, or N(C₁₋₈-alkyl)₂; unsubstituted or mono- or poly-substituted phenyl or heteroaryl; or unsubstituted or mono- or poly-substituted C₂-alkyl-bridged phenyl or heteroaryl, wherein the alkyl chain optionally may be branched or unbranched, saturated or unsaturated, unsubstituted or mono- or poly-substituted.

4. A compound as claimed in claim 1, wherein R¹ and R² each denote H; or R² denotes H and R¹ is not H.

5. A compound as claimed in claim 1, corresponding to one of the formulas (1a), (1b) or (1c): 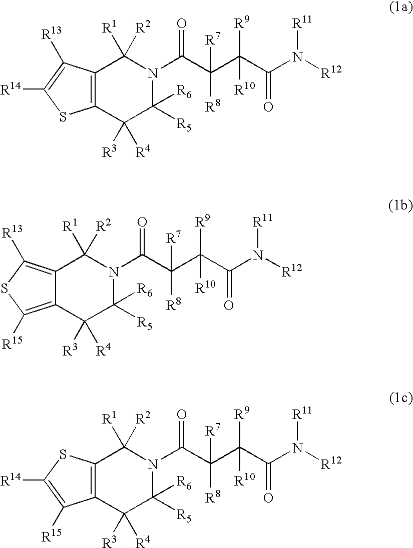
wherein R¹³, R¹⁴ and R¹⁵ each independently denote H; F; Cl; Br; I; NO; NO₂; CN; NH₂, NH—C₁₋₈-alkyl; N(C₁₋₈-alkyl)₂; NH—C(═O)C₁₋₈-alkyl; NH—C(═O)-aryl; NH—C(═O)-heteroaryl; C₁₋₈-alkyl; CF₃; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-O; O—(C₁₋₈-alkyl)-O—C₁₋₈alkyl; O-benzyl; O-aryl; O-heteroaryl; O—C(═O)C₁₋₈-alkyl; O—C(═O)aryl; O—C(═O)heteroaryl; SH; S—C₁₋₈-alkyl; SCF₃; S-benzyl; S-aryl; S-heteroaryl; aryl; heteroaryl; C₃₋₇-cycloalkyl; heterocyclyl, or C₁₋₈-alkyl-bridged aryl, heteroaryl, C₃₋₇-cycloalkyl or heterocyclyl.

6. A compound as claimed in claim 1, wherein R³, R⁴, R⁵ and R⁶ each independently represent H; saturated or unsaturated, branched or unbranched, unsubstituted or mono- or poly-substituted C₁₋₈-alkyl; or unsubstituted or mono- or poly-substituted phenyl.

7. A compound as claimed in claim 1, wherein
R⁸ represents H; or saturated or unsaturated, branched or unbranched, unsubstituted or mono- or poly-substituted C₁₋₈-alkyl; and
R¹⁰ represents H; or saturated or unsaturated, branched or unbranched, unsubstituted or mono- or poly-substituted C₁₋₈-alkyl.

8. A compound as claimed in claim 1, wherein R¹¹ represents H; saturated or unsaturated, branched or unbranched C₁₋₈-alkyl; saturated or unsaturated C₃₋₇-cycloalkyl; or unsubstituted or mono- or poly-substituted benzyl.

9. A compound as claimed in claim 1, wherein R¹² represents saturated or unsaturated, branched or unbranched, unsubstituted or mono- or poly-substituted C₄₋₁₆-alkyl; or corresponds to one of the following formulas A, B and C: 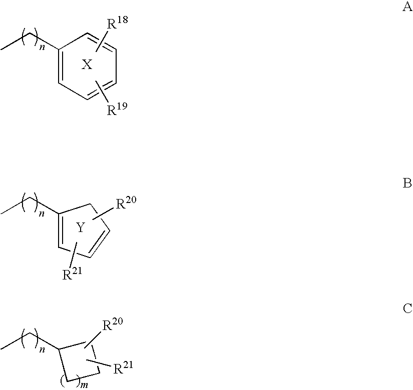
wherein
n is 0, 1, 2, 3, 4, 5, 6, 7 or 8;
m is 0, 1, 2 or 3;
the ring X can contain one or two N atoms as ring member(s);
the ring Y contains at least 1 heteroatom selected from N, O and S and can contain up to 3 heteroatoms each independently selected from N, O and S;
R¹⁸ and R¹⁹ each independently denote H; F; Cl; Br; I; NO; NO₂; CF₃; CN; R⁰; C(═O)H; C(═O)R⁰; CO₂H; C(═O)OR⁰; CONH₂; C(═O)NHR⁰; C(═O)N(R⁰)₂; OH; OR⁰; O—(C₁₋₈-alkyl)-O; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰;S(═O)R⁰; S(═O)₂R⁰; S(═O)₂OH; S(═O)₂OR⁰; S(═O)₂NH₂; S(═O)₂NHR⁰; or S(═O)₂N(R⁰)₂; or
R¹⁸ and R¹⁹, together with the carbon or nitrogen atoms connecting them form an unsubstituted or mono- or poly-substituted aryl or heteroaryl ring fused with the phenyl ring; or a saturated or unsaturated, unsubstituted or mono- or poly-substituted C₃₋₇-cycloalkyl or heterocyclyl ring fused with the phenyl ring; and
R²⁰ and R²¹ each independently denote H; saturated or unsaturated, branched or unbranched, unsubstituted or mono- or poly-substituted C₁₋₈-alkyl; or saturated or unsaturated, unsubstituted or mono- or poly-substituted C₃₋₇-cycloalkyl or heterocyclyl.

10. A compound as claimed in claim 1, wherein R¹¹ and R¹² together with the nitrogen atom to which they are attached form a ring corresponding to one of the following formulas: 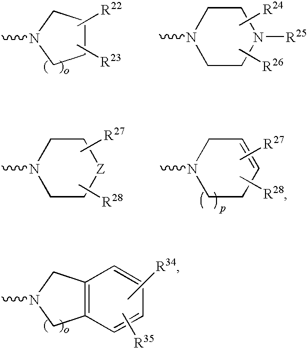
wherein
Z represents O or S;
o is 0, 1 or 2;
p is 0 or 1;
R²², R²³, R²⁴, R²⁵ and R²⁶ each independently denote H; saturated or unsaturated, branched or unbranched, unsubstituted or mono- or poly-substituted C₁₋₈-alkyl; saturated or unsaturated, unsubstituted or mono- or poly-substituted C₃₋₇-cycloalkyl or heterocyclyl; unsubstituted or mono- or poly-substituted aryl or heteroaryl; saturated or unsaturated, unsubstituted or mono- or poly-substituted C₁₋₈-alkyl-bridged C₃₋₇-cycloalkyl or heterocyclyl, wherein the alkyl chain optionally may be branched or unbranched, saturated or unsaturated, or unsubstituted, mono- or poly-substituted; or unsubstituted or mono- or poly-substituted C₁₋₈-alkyl-bridged aryl or heteroaryl, wherein the alkyl chain optionally may be branched or unbranched, saturated or unsaturated, mono- or poly-substituted; or
R²² and R²³ together with the carbon atom(s) connecting them form a saturated or unsaturated, unsubstituted or mono- or poly-substituted C₃₋₇-cycloalkyl or heterocyclyl ring optionally fused with unsubstituted or mono- or poly-substituted aryl or heteroaryl; or form a fused unsubstituted or mono- or poly-substituted aryl or heteroaryl ring;
R²⁷ and R²⁸ each independently denote H; saturated or unsaturated; branched or unbranched, unsubstituted or mono- or poly-substituted C₁₋₈-alkyl; and
R³⁴ and R³⁵ each independently denote H; F; Cl; Br; I; NO; NO₂; CN; NH₂; NH—C₁₋₈-alkyl; N(C₁₋₈-alkyl)₂; NH—C(═O)C₁₋₈-alkyl; NH—C(═O)-aryl; NH—C(═O)-heteroaryl; C₁₋₈-alkyl; CF₃; C(═O)H; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(aryl)(heteroaryl); OH; O—C₁₋₈-alkyl; O—C₁₋₈-alkyl-OH; OCF₃; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; SH; S—C₁₋₈-alkyl; SCF₃; S-benzyl; S-aryl; S-heteroaryl; aryl; heteroaryl; C₃₋₇-cycloalkyl; heterocyclyl; or C₁₋₈-alkyl-bridged aryl, heteroaryl, C₃₋₇-cycloalkyl or heterocyclyl.

11. A compound as claimed in claim 1, selected from the group consisting of:
1 4-oxo-4-(4-(thiophen-2-yl)-6,7-dihydrothieno[3,2-c]pyridin-5(4H)-yl)-N-(3-(trifluoromethyl)benzyl)butanamide;
2 4-oxo-4-(4-phenyl-6,7-dihydrothieno[3,2-c]pyridin-5(4H)-yl)-N-(3-(trifluoromethyl)benzyl)butanamide;
3 4-(4-(3-fluorophenyl)-6,7-dihydrothieno[3,2-c]pyridin-5(4H)-yl)-4-oxo-N-(3-(trifluoromethyl)benzyl)butanamide;
4 4-oxo-4-(4-m-tolyl-6,7-dihydrothieno[3,2-c]pyridin-5(4H)-yl)-N-(3-(trifluoromethyl)benzyl)butanamide;
5 4-oxo-4-(4-p-tolyl-6,7-dihydrothieno[3,2-c]pyridin-5(4H)-yl)-N-(3-(trifluoromethyl)benzyl)butanamide;
6 4-(4-(2-fluorophenyl)-6,7-dihydrothieno[3,2-c]pyridin-5(4H)-yl)-4-oxo-N-(3-(trifluoromethyl)benzyl)butanamide;
7 4-(4-(4-fluorophenyl)-6,7-dihydrothieno[3,2-c]pyridin-5(4H)-yl)-4-oxo-N-(3-(trifluoromethyl)benzyl)butanamide;
8 4-oxo-4-(4-o-tolyl-6,7-dihydrothieno[3,2-c]pyridin-5(4H)-yl)-N-(3-(trifluoromethyl)benzyl)butanamide;
9 4-oxo-4-(7-phenyl-4,5-dihydrothieno[2,3-c]pyridin-6(7H)-yl)-N-(3-(trifluoromethyl)benzyl)butanamide;
10 4-(2-methyl-4-phenyl-6,7-dihydrothieno[3,2-c]pyridin-5(4H)-yl)-4-oxo-N-(3-(trifluoromethyl)benzyl)butanamide;
11 N-(4-methylbenzyl)-4-oxo-4-(4-phenyl-6,7-dihydrothieno[3,2-c]pyridin-5(4H)-yl)butanamide;
12 N-benzyl-4-oxo-4-(4-phenyl-6,7-dihydrothieno[3,2-c]pyridin-5(4H)-yl)butanamide;
13 4-oxo-4-(4-phenyl-6,7-dihydrothieno[3,2-c]pyridin-5(4H)-yl)-N-(4-(trifluoromethyl)benzyl)butanamide;
14 N-(2-methoxybenzyl)-4-oxo-4-(4-phenyl-6,7-dihydrothieno[3,2-c]pyridin-5(4H)-yl)butanamide;
15 N-(3-methoxybenzyl)-4-oxo-4-(4-phenyl-6,7-dihydrothieno[3,2-c]pyridin-5(4H)-yl)butanamide;
16 N-(4-methoxybenzyl)-4-oxo-4-(4-phenyl-6,7-dihydrothieno[3,2-c]pyridin-5(4H)-yl)butanamide;
17 4-oxo-4-(4-phenyl-6,7-dihydrothieno[3,2-c]pyridin-5(4H)-yl)-N-(2-(trifluoromethyl)benzyl)butanamide;
18 N-(3-fluorobenzyl)-4-oxo-4-(4-phenyl-6,7-dihydrothieno[3,2-c]pyridin-5(4H)-yl)butanamide;
19 N-(3-methylbenzyl)-4-oxo-4-(4-phenyl-6,7-dihydrothieno[3,2-c]pyridin-5(4H)-yl)butanamide;
20 4-(3-methyl-4-phenyl-6,7-dihydrothieno[3,2-c]pyridin-5(4H)-yl)-4-oxo-N-(3-(trifluoromethyl)benzyl)butanamide;
21 4-(4-cyclohexyl-6,7-dihydrothieno[3,2-c]pyridin-5(4H)-yl)-4-oxo-N-(3-(trifluoromethyl)benzyl)butanamide;
22 4-(4-isopropyl-6,7-dihydrothieno[3,2-c]pyridin-5(4H)-yl)-4-oxo-N-(3-(trifluoromethyl)benzyl)butanamide;
23 4-(4-butyl-6,7-dihydrothieno[3,2-c]pyridin-5(4H)-yl)-4-oxo-N-(3-(trifluoromethyl)benzyl)butanamide;
24 N-methyl-4-oxo-4-(4-phenyl-6,7-dihydrothieno[3,2-c]pyridin-5(4H)-yl)-N-(3-(trifluoromethyl)benzyl)butanamide;
25 4-oxo-N-phenethyl-4-(4-phenyl-6,7-dihydrothieno[3,2-c]pyridin-5(4H)-yl)butanamide;
26 N-(2-methylbenzyl)-4-oxo-4-(4-phenyl-6,7-dihydrothieno[3,2-c]pyridin-5(4H)-yl)butanamide;
27 4-oxo-2-phenyl-4-(4-phenyl-6,7-dihydrothieno[3,2-c]pyridin-5(4H)-yl)-N-(3-(trifluoromethyl)benzyl)butanamide;
28 (R)-2-methyl-4-oxo-4-(4-phenyl-6,7-dihydrothieno[3,2-c]pyridin-5(4H)-yl)-N-(3-(trifluoromethyl)benzyl)butanamide;
29 4-oxo-3-phenyl-4-(4-phenyl-6,7-dihydrothieno[3,2-c]pyridin-5(4H)-yl)-N-(3-(trifluoromethyl)benzyl)butanamide;
30 4-(6,7-dihydrothieno[3,2-c]pyridin-5(4H)-yl)-4-oxo-3-phenyl-N-(3-(trifluoromethyl)benzyl)butanamide;
31 rac. (1S,2R)-2-(4-phenyl-4,5,6,7-tetrahydrothieno[3,2-c]pyridine-5-carbonyl)-N-(3-(trifluoromethyl)benzyl)cyclohexanecarboxamide;
32 rac. (1S)-2-(4-phenyl-4,5,6,7-tetrahydrothieno[3,2-c]pyridine-5-carbonyl)-N-(3-(trifluoromethyl)benzyl)cyclohexanecarboxamide;
33 rac. (1S,2R)-2-(4-phenyl-4,5,6,7-tetrahydrothieno[3,2-c]pyridine-5-carbonyl)-N-(3-(trifluoromethyl)benzyl)cyclopentanecarboxamide;
34 (R)-3-methyl-4-oxo-4-(4-phenyl-6,7-dihydrothieno[3,2-c]pyridin-5(4H)-yl)-N-(3-(trifluoromethyl)benzyl)butanamide;
35 (−)-4-oxo-4-(4-phenyl-6,7-dihydrothieno[3,2-c]pyridin-5(4H)-yl)-N-(3-(trifluoromethyl)benzyl)butanamide;
36 (+)-4-oxo-4-(4-phenyl-6,7-dihydrothieno[3,2-c]pyridin-5(4H)-yl)-N-(3-(trifluoromethyl)benzyl)butanamide;
37 4-(6,7-dihydrothieno[3,2-c]pyridin-5(4H)-yl)-4-oxo-2-phenyl-N-(3-(trifluoromethyl)benzyl)butanamide;
38 N-(3,5-bis(trifluoromethyl)benzyl)-4-oxo-4-(4-phenyl-6,7-dihydrothieno[3,2-c]-pyridin-5(4H)-yl)butanamide;
39 N-(2-fluoro-5-(trifluoromethyl)benzyl)-4-oxo-4-(4-phenyl-6,7-dihydrothieno-[3,2-c]pyridin-5(4H)-yl)butanamide;
40 N-(2-fluoro-3-(trifluoromethyl)benzyl)-4-oxo-4-(4-phenyl-6,7-dihydrothieno-[3,2-c]pyridin-5(4H)-yl)butanamide;
41 N-(3-fluoro-5-(trifluoromethyl)benzyl)-4-oxo-4-(4-phenyl-6,7-dihydrothieno-[3,2-c]pyridin-5(4H)-yl)butanamide;
42 rac. (1S,2R)-2-(4-phenyl-4,5,6,7-tetrahydrothieno[3,2-c]pyridine-5-carbonyl)-N-(3-(trifluoromethyl)benzyl)cyclopropanecarboxamide;
43 N-(3,4-difluorobenzyl)-4-oxo-4-(4-phenyl-6,7-dihydrothieno[3,2-c]pyridin-5(4H)-yl)butanamide;
44 4-oxo-4-(5-phenyl-4,5-dihydrothieno[2,3-c]pyridin-6(7H)-yl)-N-(3-(trifluoromethyl)benzyl)butanamide;
45 4-(1-methyl-4-phenyl-6,7-dihydrothieno[3,4-c]pyridin-5(4H)-yl)-4-oxo-N-(3-(trifluoromethyl)benzyl)butanamide;
46 N-(4-methoxyphenyl)-4-oxo-4-(4-p-tolyl-6,7-dihydrothieno[3,2-c]pyridin-5(4H)-yl)butanamide;
47 N-(1-methyl-1H-indazol-6-yl)-4-oxo-4-(4-p-tolyl-6,7-dihydrothieno[3,2-c]pyridin-5(4H)-yl)butanamide;
48 N-benzyl-4-oxo-4-(4-p-tolyl-6,7-dihydrothieno[3,2-c]pyridin-5(4H)-yl)butanamide;
49 4-oxo-N-phenethyl-4-(4-p-tolyl-6,7-dihydrothieno[3,2-c]pyridin-5(4H)-yl)butanamide;
50 4-oxo-N-(pyridin-4-ylmethyl)-4-(4-p-tolyl-6,7-dihydrothieno[3,2-c]pyridin-5(4H)-yl)butanamide;
51 4-oxo-N-(3-phenylpropyl)-4-(4-p-tolyl-6,7-dihydrothieno[3,2-c]pyridin-5(4H)-yl)butanamide;
52 N-(benzo[c][1,2,5]thiadiazol-4-yl)-4-oxo-4-(4-m-tolyl-6,7-dihydrothieno[3,2-c]-pyridin-5(4H)-yl)butanamide;
53 N-(1-methyl-1H-indazol-6-yl)-4-oxo-4-(4-m-tolyl-6,7-dihydrothieno[3,2-c]-pyridin-5(4H)-yl)butanamide;
54 4-oxo-N-(pyridin-2-ylmethyl)-4-(4-m-tolyl-6,7-dihydrothieno[3,2-c]pyridin-5(4H)-yl)butanamide;
55 1-(4-methylpiperazin-1-yl)-4-(4-m-tolyl-6,7-dihydrothieno[3,2-c]pyridin-5(4H)-yl)butane-1,4-dione;
56 3-methyl-4-oxo-4-(4-phenyl-6,7-dihydrothieno[3,2-c]pyridin-5(4H)-yl)-N-(pyridin-2-ylmethyl)butanamide;
57 4-oxo-4-(4-phenyl-6,7-dihydrothieno[3,2-c]pyridin-5(4H)-yl)-N-(thiophen-2-ylmethyl)butanamide;
58 N-(2-chlorophenyl)-4-(4-(4-fluorophenyl)-6,7-dihydrothieno[3,2-c]pyridin-5(4H)-yl)-4-oxobutanamide;
59 4-(4-(4-chlorophenyl)-6,7-dihydrothieno[3,2-c]pyridin-5(4H)-yl)-N-(furan-2-ylmethyl)-4-oxobutanamide;
60 4-(4-(4-chlorophenyl)-6,7-dihydrothieno[3,2-c]pyridin-5(4H)-yl)-4-oxo-N-propylbutanamide;
61 N-(2-chlorobenzyl)-4-oxo-4-(4-m-tolyl-6,7-dihydrothieno[3,2-c]pyridin-5(4H)-yl)butanamide;
62 N-(2,4-dichlorobenzyl)-4-(4-(3-fluorophenyl)-6,7-dihydrothieno[3,2-c]pyridin-5(4H)-yl)-4-oxobutanamide;
63 N-(4-fluorobenzyl)-4-(4-(3-fluorophenyl)-6,7-dihydrothieno[3,2-c]pyridin-5(4H)-yl)-4-oxobutanamide;
64 N-(3,4-dichlorobenzyl)-3-methyl-4-oxo-4-(4-phenyl-6,7-dihydrothieno[3,2-c]-pyridin-5(4H)-yl)butanamide;
65 N-(2,5-difluorobenzyl)-3-methyl-4-oxo-4-(4-phenyl-6,7-dihydrothieno[3,2-c]-pyridin-5(4H)-yl)butanamide;
66 3-methyl-4-oxo-4-(4-phenyl-6,7-dihydrothieno[3,2-c]pyridin-5(4H)-yl)-N-(3-(trifluoromethyl)benzyl)butanamide;
67 1-(4-(4-chlorophenyl)-6,7-dihydrothieno[3,2-c]pyridin-5(4H)-yl)-4-morpholino-butane-1,4-dione;
68 1-(4-(4-chlorophenyl)-6,7-dihydrothieno[3,2-c]pyridin-5(4H)-yl)-4-(4-(4-fluorophenyl)piperazin-1-yl)butane-1,4-dione;
69 2-methyl-N-(2-(5-methyl-1H-pyrazol-1-yl)ethyl)-4-oxo-4-(4-phenyl-6,7-dihydro-thieno[3,2-c]pyridin-5(4H)-yl)butanamide;
70 N-(naphthalen-1-ylmethyl)-4-oxo-4-(4-phenyl-6,7-dihydrothieno[3,2-c]pyridin-5(4H)-yl)butanamide;
71 4-(4-(4-fluorophenyl)-6,7-dihydrothieno[3,2-c]pyridin-5(4H)-yl)-N-(4-methyl-benzyl)-4-oxobutanamide;
72 N-(benzo[d][1,3]dioxol-5-ylmethyl)-4-(4-(4-fluorophenyl)-6,7-dihydrothieno-[3,2-c]pyridin-5(4H)-yl)-4-oxobutanamide;
73 4-(4-(4-fluorophenyl)-6,7-dihydrothieno[3,2-c]pyridin-5(4H)-yl)-4-oxo-N-(2-(trifluoromethyl)benzyl)butanamide;
74 4-(4-(4-fluorophenyl)-6,7-dihydrothieno[3,2-c]pyridin-5(4H)-yl)-N-(naphthalen-1-ylmethyl)-4-oxobutanamide;
75 4-(4-(4-chlorophenyl)-6,7-dihydrothieno[3,2-c]pyridin-5(4H)-yl)-N-(3-fluorobenzyl)-4-oxobutanamide;
76 2-methyl-N-(1-methyl-1H-indazol-6-yl)-4-oxo-4-(4-phenyl-6,7-dihydrothieno-[3,2-c]pyridin-5(4H)-yl)butanamide;
77 N-(4-methoxybenzyl)-2-methyl-4-oxo-4-(4-phenyl-6,7-dihydrothieno[3,2-c]-pyridin-5(4H)-yl)butanamide;
78 2-methyl-1-(4-methylpiperazin-1-yl)-4-(4-phenyl-6,7-dihydrothieno[3,2-c]-pyridin-5(4H)-yl)butane-1,4-dione;
79 N-(2-(1H-indol-3-yl)ethyl)-2-methyl-4-oxo-4-(4-phenyl-6,7-dihydrothieno[3,2-c]-pyridin-5(4H)-yl)butanamide;
80 N-(2-fluorobenzyl)-2-methyl-4-oxo-4-(4-phenyl-6,7-dihydrothieno[3,2-c]pyridin-5(4H)-yl)butanamide;
81 2-methyl-4-oxo-4-(4-phenyl-6,7-dihydrothieno[3,2-c]pyridin-5(4H)-yl)-N-(2-(trifluoromethyl)benzyl)butanamide;
82 4-oxo-N-(2-(piperidin-1-yl)ethyl)-4-(4-p-tolyl-6,7-dihydrothieno[3,2-c]pyridin-5(4H)-yl)butanamide;
83 4-oxo-N-((tetrahydrofuran-2-yl)methyl)-4-(4-p-tolyl-6,7-dihydrothieno[3,2-c]-pyridin-5(4H)-yl)butanamide;
84 N-(4-chlorobenzyl)-4-oxo-4-(4-p-tolyl-6,7-dihydrothieno[3,2-c]pyridin-5(4H)-yl)butanamide;
85 N-(2,3-dichlorobenzyl)-4-oxo-4-(4-p-tolyl-6,7-dihydrothieno[3,2-c]pyridin-5(4H)-yl)butanamide;
86 4-oxo-N-(2-(thiophen-2-yl)ethyl)-4-(4-m-tolyl-6,7-dihydrothieno[3,2-c]pyridin-5(4H)-yl)butanamide;
87 N-(cyclohexylmethyl)-4-oxo-4-(4-m-tolyl-6,7-dihydrothieno[3,2-c]pyridin-5(4H)-yl)butanamide;
88 N-(3-chlorophenethyl)-4-oxo-4-(4-m-tolyl-6,7-dihydrothieno[3,2-c]pyridin-5(4H)-yl)butanamide;
89 N-(3,3-diphenylpropyl)-4-oxo-4-(4-m-tolyl-6,7-dihydrothieno[3,2-c]pyridin-5(4H)-yl)butanamide;
90 4-(4-(3-fluorophenyl)-6,7-dihydrothieno[3,2-c]pyridin-5(4H)-yl)-N-(3-morpholinopropyl)-4-oxobutanamide;
91 4-(4-(3-fluorophenyl)-6,7-dihydrothieno[3,2-c]pyridin-5(4H)-yl)-4-oxo-N-(pyridin-3-ylmethyl)butanamide;
92 4-(4-(3-fluorophenyl)-6,7-dihydrothieno[3,2-c]pyridin-5(4H)-yl)-N-(3-methyl-benzyl)-4-oxobutanamide;
93 4-(4-(3-fluorophenyl)-6,7-dihydrothieno[3,2-c]pyridin-5(4H)-yl)-N-(4-methylphenethyl)-4-oxobutanamide;
94 3-methyl-4-oxo-4-(4-phenyl-6,7-dihydrothieno[3,2-c]pyridin-5(4H)-yl)-N-(4-phenylbutyl)butanamide;
95 N-(biphenyl-4-ylmethyl)-4-oxo-4-(4-m-tolyl-6,7-dihydrothieno[3,2-c]pyridin-5(4H)-yl)butanamide;
96 4-(4-(3-fluorophenyl)-6,7-dihydrothieno[3,2-c]pyridin-5(4H)-yl)-N-(2-methoxybenzyl)-4-oxobutanamide;
97 N-(4-chlorophenethyl)-4-(4-(3-fluorophenyl)-6,7-dihydrothieno[3,2-c]pyridin-5(4H)-yl)-4-oxobutanamide;
98 4-(4-(3-fluorophenyl)-6,7-dihydrothieno[3,2-c]pyridin-5(4H)-yl)-N-((5-methyl-3-phenylisoxazol-4-yl)methyl)-4-oxobutanamide;
99 N-(2,6-difluorobenzyl)-3-methyl-4-oxo-4-(4-phenyl-6,7-dihydrothieno[3,2-c]-pyridin-5(4H)-yl)butanamide;
100 N-(3,5-difluorobenzyl)-3-methyl-4-oxo-4-(4-phenyl-6,7-dihydrothieno[3,2-c]-pyridin-5(4H)-yl)butanamide;
101 N-(3-chlorobenzyl)-3-methyl-4-oxo-4-(4-phenyl-6,7-dihydrothieno[3,2-c]pyridin-5(4H)-yl)butanamide;
102 N-(3,5-dimethoxyphenethyl)-3-methyl-4-oxo-4-(4-phenyl-6,7-dihydrothieno-[3,2-c]pyridin-5(4H)-yl)butanamide;
103 N-(3,4-difluorobenzyl)-4-oxo-4-(4-phenyl-6,7-dihydrothieno[3,2-c]pyridin-5(4H)-yl)butanamide;
104 N-(2,4-dichlorophenethyl)-4-oxo-4-(4-phenyl-6,7-dihydrothieno[3,2-c]pyridin-5(4H)-yl)butanamide;
105 N-(2-(1H-1,2,4-triazol-1-yl)ethyl)-4-oxo-4-(4-phenyl-6,7-dihydrothieno[3,2-c]-pyridin-5(4H)-yl)butanamide;
106 4-(4-(4-fluorophenyl)-6,7-dihydrothieno[3,2-c]pyridin-5(4H)-yl)-N-(2-methyl-benzyl)-4-oxobutanamide;
107 N-(4-fluorophenethyl)-4-(4-(4-fluorophenyl)-6,7-dihydrothieno[3,2-c]pyridin-5(4H)-yl)-4-oxobutanamide;
108 4-(4-(4-fluorophenyl)-6,7-dihydrothieno[3,2-c]pyridin-5(4H)-yl)-N-((5-methyl-isoxazol-3-yl)methyl)-4-oxobutanamide;
109 N-(2-chlorophenethyl)-4-(4-(4-fluorophenyl)-6,7-dihydrothieno[3,2-c]pyridin-5(4H)-yl)-4-oxobutanamide;
110 4-(4-(4-chlorophenyl)-6,7-dihydrothieno[3,2-c]pyridin-5(4H)-yl)-N-(2-cyclo-hexenylethyl)-4-oxobutanamide;
111 4-(4-(4-chlorophenyl)-6,7-dihydrothieno[3,2-c]pyridin-5(4H)-yl)-N-(3,5-dimethoxybenzyl)-4-oxobutanamide;
112 4-(4-(4-chlorophenyl)-6,7-dihydrothieno[3,2-c]pyridin-5(4H)-yl)-N-(3,4-dichlorophenethyl)-4-oxobutanamide;
113 4-(4-(4-chlorophenyl)-6,7-dihydrothieno[3,2-c]pyridin-5(4H)-yl)-N-(3-fluorophenethyl)-4-oxobutanamide;
114 1-(3-phenylpiperidin-1-yl)-4-(4-p-tolyl-6,7-dihydrothieno[3,2-c]pyridin-5(4H)-yl)butane-1,4-dione;
115 1-(4-benzylpiperazin-1-yl)-4-(4-p-tolyl-6,7-dihydrothieno[3,2-c]pyridin-5(4H)-yl)butane-1,4-dione;
116 1-(4-(4-methoxyphenyl)piperazin-1-yl)-4-(4-p-tolyl-6,7-dihydrothieno[3,2-c]-pyridin-5(4H)-yl)butane-1,4-dione;
117 1-(2-benzylpiperidin-1-yl)-4-(4-m-tolyl-6,7-dihydrothieno[3,2-c]pyridin-5(4H)-yl)butane-1,4-dione;
118 1-(4-(2-fluorophenyl)piperazin-1-yl)-4-(4-m-tolyl-6,7-dihydrothieno[3,2-c]-pyridin-5(4H)-yl)butane-1,4-dione;
119 1-(4-(cyclohexylmethyl)piperazin-1-yl)-4-(4-m-tolyl-6,7-dihydrothieno[3,2-c]-pyridin-5(4H)-yl)butane-1,4-dione;
120 N-(3-(1H-imidazol-1-yl)propyl)-4-(4-(3-fluorophenyl)-6,7-dihydrothieno[3,2-c]-pyridin-5(4H)-yl)-4-oxobutanamide;
121 1-(4-(3-fluorophenyl)-6,7-dihydrothieno[3,2-c]pyridin-5(4H)-yl)-4-(3-phenyl-pyrrolidin-1-yl)butane-1,4-dione;
122 1-(4-(3-fluorophenyl)-6,7-dihydrothieno[3,2-c]pyridin-5(4H)-yl)-4-(4-(furan-2-carbonyl)piperazin-1-yl)butane-1,4-dione;
123 N-(3-(3,4-dihydroquinolin-1(2H)-yl)propyl)-3-methyl-4-oxo-4-(4-phenyl-6,7-dihydrothieno[3,2-c]pyridin-5(4H)-yl)butanamide;
124 2-methyl-1-(4-phenyl-6,7-dihydrothieno[3,2-c]pyridin-5(4H)-yl)-4-(piperidin-1-yl)butane-1,4-dione;
125 4-(4-(2-methoxyphenyl)piperazin-1-yl)-2-methyl-1-(4-phenyl-6,7-dihydrothieno-[3,2-c]pyridin-5(4H)-yl)butane-1,4-dione;
126 4-oxo-4-(4-phenyl-6,7-dihydrothieno[3,2-c]pyridin-5(4H)-yl)-N-(2-(pyrazin-2-yl)ethyl)butanamide;
127 N,N-diethyl-4-oxo-4-(4-phenyl-6,7-dihydrothieno[3,2-c]pyridin-5(4H)-yl)-butanamide;
128 1-(4-phenyl-6,7-dihydrothieno[3,2-c]pyridin-5(4H)-yl)-4-(4-(pyridin-2-yl)piperazin-1-yl)butane-1,4-dione;
129 1-(4-(4-fluorophenyl)-6,7-dihydrothieno[3,2-c]pyridin-5(4H)-yl)-4-(4-(3-methoxyphenyl)piperazin-1-yl)butane-1,4-dione;
130 1-(4-isopropylpiperazin-1-yl)-4-(4-p-tolyl-6,7-dihydrothieno[3,2-c]pyridin-5(4H)-yl)butane-1,4-dione;
131 1-(4-p-tolyl-6,7-dihydrothieno[3,2-c]pyridin-5(4H)-yl)-4-(4-(3-(trifluoromethyl)-phenyl)piperazin-1-yl)butane-1,4-dione;
132 1-(4-phenylpiperazin-1-yl)-4-(4-m-tolyl-6,7-dihydrothieno[3,2-c]pyridin-5(4H)-yl)butane-1,4-dione;
133 1-(3,5-dimethylpiperidin-1-yl)-4-(4-m-tolyl-6,7-dihydrothieno[3,2-c]pyridin-5(4H)-yl)butane-1,4-dione;
134 1-(5,6-dihydropyridin-1(2H)-yl)-4-(4-(3-fluorophenyl)-6,7-dihydrothieno[3,2-c]-pyridin-5(4H)-yl)butane-1,4-dione;
135 1-(4-(3-fluorophenyl)-6,7-dihydrothieno[3,2-c]pyridin-5(4H)-yl)-4-(4-(pyrimidin-2-yl)piperazin-1-yl)butane-1,4-dione;
136 1-(4-(3-fluorophenyl)-6,7-dihydrothieno[3,2-c]pyridin-5(4H)-yl)-4-(3-methyl-piperidin-1-yl)butane-1,4-dione;
137 N-ethyl-4-(4-(3-fluorophenyl)-6,7-dihydrothieno[3,2-c]pyridin-5(4H)-yl)-4-oxo-N-(pyridin-4-ylmethyl)butanamide;
138 4-(4-acetylpiperazin-1-yl)-2-methyl-1-(4-phenyl-6,7-dihydrothieno[3,2-c]pyridin-5(4H)-yl)butane-1,4-dione;
139 4-(2,6-dimethylmorpholino)-2-methyl-1-(4-phenyl-6,7-dihydrothieno[3,2-c]-pyridin-5(4H)-yl)butane-1,4-dione;
140 4-(2,5-dihydro-1H-pyrrol-1-yl)-2-methyl-1-(4-phenyl-6,7-dihydrothieno[3,2-c]-pyridin-5(4H)-yl)butane-1,4-dione;
141 N-(2-methoxyphenethyl)-4-oxo-4-(4-phenyl-6,7-dihydrothieno[3,2-c]pyridin-5(4H)-yl)butanamide;
142 4-(4-(4-fluorophenyl)-6,7-dihydrothieno[3,2-c]pyridin-5(4H)-yl)-N-isopentyl-4-oxobutanamide;
143 N-(2,4-dimethoxybenzyl)-4-(4-(4-fluorophenyl)-6,7-dihydrothieno[3,2-c]pyridin-5(4H)-yl)-4-oxobutanamide;
144 4-(4-(4-chlorophenyl)-6,7-dihydrothieno[3,2-c]pyridin-5(4H)-yl)-N-((5-methyl-furan-2-yl)methyl)-4-oxobutanamide;
145 4-(4-(4-chlorophenyl)-6,7-dihydrothieno[3,2-c]pyridin-5(4H)-yl)-4-oxo-N-pentylbutanamide;
146 1-(4-benzylpiperidin-1-yl)-4-(4-(4-chlorophenyl)-6,7-dihydrothieno[3,2-c]-pyridin-5(4H)-yl)butane-1,4-dione;
147 1-(4-(4-chlorophenyl)-6,7-dihydrothieno[3,2-c]pyridin-5(4H)-yl)-4-(4-methyl-piperidin-1-yl)butane-1,4-dione;
148 1-(azetidin-1-yl)-4-(4-(4-chlorophenyl)-6,7-dihydrothieno[3,2-c]pyridin-5(4H)-yl)butane-1,4-dione;
149 1-(3,4-dihydroisoquinolin-2(1H)-yl)-2-methyl-4-(4-phenyl-6,7-dihydrothieno-[3,2-c]pyridin-5(4H)-yl)butane-1,4-dione;
150 1-(4-(benzo[d][1,3]dioxol-5-ylmethyl)piperazin-1-yl)-2-methyl-4-(4-phenyl-6,7-dihydrothieno[3,2-c]pyridin-5(4H)-yl)butane-1,4-dione;
151 N-benzyl-N-ethyl-4-oxo-4-(4-p-tolyl-6,7-dihydrothieno[3,2-c]pyridin-5(4H)-yl)butanamide;
152 N-methyl-4-oxo-N-phenethyl-4-(4-p-tolyl-6,7-dihydrothieno[3,2-c]pyridin-5(4H)-yl)butanamide;
153 ethyl 1-(4-oxo-4-(4-p-tolyl-6,7-dihydrothieno[3,2-c]pyridin-5(4H)-yl)butanoyl)piperidine-4-carboxylate;
154 (E)-1-(4-(3-phenylprop-2-enyl)-piperazin-1-yl)-4-(4-p-tolyl-6,7-dihydrothieno-[3,2-c]pyridin-5(4H)-yl)butane-1,4-dione;
155 1-(2-(thiazol-2-yl)pyrrolidin-1-yl)-4-(4-p-tolyl-6,7-dihydrothieno[3,2-c]pyridin-5(4H)-yl)butane-1,4-dione;
156 N-cyclohexyl-N-ethyl-4-oxo-4-(4-m-tolyl-6,7-dihydrothieno[3,2-c]pyridin-5(4H)-yl)butanamide;
157 N-ethyl-N-isopropyl-4-oxo-4-(4-m-tolyl-6,7-dihydrothieno[3,2-c]pyridin-5(4H)-yl)butanamide;
158 1-(pyrrolidin-1-yl)-4-(4-m-tolyl-6,7-dihydrothieno[3,2-c]pyridin-5(4H)-yl)butane-1,4-dione;
159 1-(4-(pyridin-4-yl)piperazin-1-yl)-4-(4-m-tolyl-6,7-dihydrothieno[3,2-c]pyridin-5(4H)-yl)butane-1,4-dione;
160 1-(2-(pyridin-2-ylmethyl)pyrrolidin-1-yl)-4-(4-m-tolyl-6,7-dihydrothieno[3,2-c]-pyridin-5(4H)-yl)butane-1,4-dione;
161 ethyl 1-(4-(4-(4-fluorophenyl)-6,7-dihydrothieno[3,2-c]pyridin-5(4H)-yl)-4-oxo-butanoyl)piperidine-3-carboxylate;
162 1-(4-(4-fluorophenyl)-6,7-dihydrothieno[3,2-c]pyridin-5(4H)-yl)-4-(4-(4-(trifluoro-methyl)phenyl)piperazin-1-yl)butane-1,4-dione;
163 1-(2-(5-bromopyridin-3-yl)pyrrolidin-1-yl)-4-(4-(4-fluorophenyl)-6,7-dihydro-thieno[3,2-c]pyridin-5(4H)-yl)butane-1,4-dione;
164 1-(4-ethylpiperazin-1-yl)-4-(4-phenyl-6,7-dihydrothieno[3,2-c]pyridin-5(4H)-yl)butane-1,4-dione;
165 1-(2-ethylpiperidin-1-yl)-4-(4-phenyl-6,7-dihydrothieno[3,2-c]pyridin-5(4H)-yl)butane-1,4-dione;
166 N,N-dibenzyl-4-oxo-4-(4-phenyl-6,7-dihydrothieno[3,2-c]pyridin-5(4H)-yl)butanamide;
167 N,N-diisobutyl-4-oxo-4-(4-phenyl-6,7-dihydrothieno[3,2-c]pyridin-5(4H)-yl)butanamide;
168 1-(3,4-dihydroquinolin-1(2H)-yl)-4-(4-(4-fluorophenyl)-6,7-dihydrothieno[3,2-c]-pyridin-5(4H)-yl)butane-1,4-dione;
169 1-(4-(3,4-dichlorobenzyl)piperazin-1-yl)-4-(4-p-tolyl-6,7-dihydrothieno[3,2-c]-pyridin-5(4H)-yl)butane-1,4-dione;
170 1-(4-p-tolyl-6,7-dihydrothieno[3,2-c]pyridin-5(4H)-yl)-4-(4-(2,4,6-trimethyl-benzyl)piperazin-1-yl)butane-1,4-dione;
171 1-(4-(4-bromobenzyl)piperazin-1-yl)-4-(4-m-tolyl-6,7-dihydrothieno[3,2-c]-pyridin-5(4H)-yl)butane-1,4-dione;
172 1-(4-(4-chlorobenzyl)piperazin-1-yl)-4-(4-m-tolyl-6,7-dihydrothieno[3,2-c]-pyridin-5(4H)-yl)butane-1,4-dione;
173 1-(2-((4,6-dimethylpyridin-2-yl)methyl)pyrrolidin-1-yl)-4-(4-(3-fluorophenyl)-6,7-dihydrothieno[3,2-c]pyridin-5(4H)-yl)butane-1,4-dione;
174 1-(4-(3-fluorophenyl)-6,7-dihydrothieno[3,2-c]pyridin-5(4H)-yl)-4-(2-((6-meythyl-pyridin-2-yl)methyl)pyrrolidin-1-yl)butane-1,4-dione;
175 1-(2-((5-ethylpyridin-2-yl)methyl)pyrrolidin-1-yl)-4-(4-(3-fluorophenyl)-6,7-dihydrothieno[3,2-c]pyridin-5(4H)-yl)butane-1,4-dione;
176 1-(4-(3-fluorophenyl)-6,7-dihydrothieno[3,2-c]pyridin-5(4H)-yl)-4-(2-(6-methoxypyridin-3-yl)piperidin-1-yl)butane-1,4-dione;
177 4-(4-(3-fluorophenyl)-6,7-dihydrothieno[3,2-c]pyridin-5(4H)-yl)-N-methyl-4-oxo-N-(pyridin-3-ylmethyl)butanamide;
178 N-(2,2-diphenylethyl)-3-methyl-4-oxo-4-(4-phenyl-6,7-dihydrothieno[3,2-c]-pyridin-5(4H)-yl)butanamide;
179 N-(cyclopropylmethyl)-4-oxo-4-(4-phenyl-6,7-dihydrothieno[3,2-c]pyridin-5(4H)-yl)butanamide;
180 1-(4-(3-fluorophenyl)-6,7-dihydrothieno[3,2-c]pyridin-5(4H)-yl)-4-(4-(4-methyl-benzyl)piperazin-1-yl)butane-1,4-dione;
181 2-methyl-1-(4-phenyl-6,7-dihydrothieno[3,2-c]pyridin-5(4H)-yl)-4-(2-(pyridin-2-ylmethyl)piperidin-1-yl)butane-1,4-dione;
182 4-(4-(3,5-dichloropyridin-4-yl)piperazin-1-yl)-2-methyl-1-(4-phenyl-6,7-dihydro-thieno[3,2-c]pyridin-5(4H)-yl)butane-1,4-dione;
183 1-(2-((4,6-dimethylpyridin-2-yl)methyl)piperidin-1-yl)-4-(4-phenyl-6,7-dihydro-thieno[3,2-c]pyridin-5(4H)-yl)butane-1,4-dione;
184 N-(4-fluorobenzyl)-N-methyl-4-oxo-4-(4-phenyl-6,7-dihydrothieno[3,2-c]pyridin-5(4H)-yl)butanamide;
185 1-(4-(3-fluorophenyl)-6,7-dihydrothieno[3,2-c]pyridin-5(4H)-yl)-4-(2-(pyridin-2-yl)pyrrolidin-1-yl)butane-1,4-dione;
186 4-(4-(4-methoxybenzyl)piperazin-1-yl)-2-methyl-1-(4-phenyl-6,7-dihydrothieno-[3,2-c]pyridin-5(4H)-yl)butane-1,4-dione;
187 2-methyl-1-(4-phenyl-6,7-dihydrothieno[3,2-c]pyridin-5(4H)-yl)-4-(3-(pyridin-3-yl)pyrrolidin-1-yl)butane-1,4-dione;
188 1-(4-(2-fluorobenzyl)piperazin-1-yl)-4-(4-phenyl-6,7-dihydrothieno[3,2-c]-pyridin-5(4H)-yl)butane-1,4-dione;
189 N-methyl-4-oxo-4-(4-phenyl-6,7-dihydrothieno[3,2-c]pyridin-5(4H)-yl)-N-(2-(pyridin-4-yl)ethyl)butanamide;
190 N-butyl-N-ethyl-3-methyl-4-oxo-4-(4-phenyl-6,7-dihydrothieno[3,2-c]pyridin-5(4H)-yl)butanamide;
191 N,3-dimethyl-4-oxo-4-(4-phenyl-6,7-dihydrothieno[3,2-c]pyridin-5(4H)-yl)-N-propylbutanamide;
192 4-((S)-2-(methoxymethyl)pyrrolidin-1-yl)-2-methyl-1-(4-phenyl-6,7-dihydro-thieno[3,2-c]pyridin-5(4H)-yl)butane-1,4-dione;
193 4-(4-(5-chloro-2-methylphenyl)piperazin-1-yl)-2-methyl-1-(4-phenyl-6,7-dihydrothieno[3,2-c]pyridin-5(4H)-yl)butane-1,4-dione;
194 N-(furan-2-ylmethyl)-N-methyl-4-oxo-4-(4-phenyl-6,7-dihydrothieno[3,2-c]-pyridin-5(4H)-yl)butanamide;
195 1-((4aR,8aS)-octahydroisoquinolin-2(1H)-yl)-4-(4-phenyl-6,7-dihydrothieno-[3,2-c]pyridin-5(4H)-yl)butane-1,4-dione;
196 1-(4-phenyl-6,7-dihydrothieno[3,2-c]pyridin-5(4H)-yl)-4-thiomorpholinobutane-1,4-dione;
197 1-(4-phenyl-6,7-dihydrothieno[3,2-c]pyridin-5(4H)-yl)-4-(2-(pyridin-3-yl)-pyrrolidin-1-yl)butane-1,4-dione;
198 N-benzyl-4-(4-(4-fluorophenyl)-6,7-dihydrothieno[3,2-c]pyridin-5(4H)-yl)-N-isopropyl-4-oxobutanamide;
199 N-benzyl-4-(4-(4-fluorophenyl)-6,7-dihydrothieno[3,2-c]pyridin-5(4H)-yl)-N-methyl-4-oxobutanamide;
200 N-(3,4-dimethoxyphenethyl)-4-(4-(4-fluorophenyl)-6,7-dihydrothieno[3,2-c]-pyridin-5(4H)-yl)-N-methyl-4-oxobutanamide;
201 1-(4-(4-fluorophenyl)-6,7-dihydrothieno[3,2-c]pyridin-5(4H)-yl)-4-(4-methyl-2-phenylpiperazin-1-yl)butane-1,4-dione;
202 1-(4-(4-chlorophenyl)-6,7-dihydrothieno[3,2-c]pyridin-5(4H)-yl)-4-(4-phenyl-piperidin-1-yl)butane-1,4-dione;
203 4-(4-(4-chlorophenyl)-6,7-dihydrothieno[3,2-c]pyridin-5(4H)-yl)-4-oxo-N-(2-(pyridin-2-yl)ethyl)butanamide;
204 N-benzyl-2-methyl-4-oxo-N-phenethyl-4-(4-phenyl-6,7-dihydrothieno[3,2-c]-pyridin-5(4H)-yl)butanamide;
205 1-(4-(4-fluorophenyl)-6,7-dihydrothieno[3,2-c]pyridin-5(4H)-yl)-4-(2-((6-methyl-pyridin-2-yl)methyl)piperidin-1-yl)butane-1,4-dione;
206 1-(4-(4-fluorophenyl)-6,7-dihydrothieno[3,2-c]pyridin-5(4H)-yl)-4-(4-(3-methyl-benzyl)piperazin-1-yl)butane-1,4-dione;
207 1-(4-(4-chlorophenyl)-6,7-dihydrothieno[3,2-c]pyridin-5(4H)-yl)-4-(2-(pyridin-4-ylmethyl)piperidin-1-yl)butane-1,4-dione;
208 1-(4-(4-chlorophenyl)-6,7-dihydrothieno[3,2-c]pyridin-5(4H)-yl)-4-(2-((5-ethyl-pyridin-2-yl)methyl)piperidin-1-yl)butane-1,4-dione;
209 1-(4-(4-chlorophenyl)-6,7-dihydrothieno[3,2-c]pyridin-5(4H)-yl)-4-(2-((3-methyl-pyridin-2-yl)methyl)piperidin-1-yl)butane-1,4-dione;
210 1-(4-(4-chlorophenyl)-6,7-dihydrothieno[3,2-c]pyridin-5(4H)-yl)-4-(4-(3-chloro-phenyl)piperazin-1-yl)butane-1,4-dione;
211 1-(4-(4-dhlorophenyl)-6,7-dihydrothieno[3,2-c]pyridin-5(4H)-yl)-4-(4-(2,3-dimethylphenyl)piperazin-1-yl)butane-1,4-dione;
212 1-(4-(4-chlorophenyl)-6,7-dihydrothieno[3,2-c]pyridin-5(4H)-yl)-4-(4-(3,4-dimethylphenyl)piperazin-1-yl)butane-1,4-dione;
213 1-(4-(4-chlorophenyl)-6,7-dihydrothieno[3,2-c]pyridin-5(4H)-yl)-4-(4-(3,4-dichlorophenyl)piperazin-1-yl)butane-1,4-dione;
214 1-(4-(4-tert-butylbenzyl)piperazin-1-yl)-4-(4-(4-chlorophenyl)-6,7-dihydro-thieno[3,2-c]pyridin-5(4H)-yl)butane-1,4-dione;
215 1-[4-(3,5-dichloro-4-pyridyl)-1-piperazinyl]-2-methyl-4-(4-phenyl-6,7-dihydro-4H-thieno[3,2-c]pyridin-5-yl)butane-1,4-dione;
216 1-[4-[(4-methoxyphenyl)methyl]-1-piperazinyl]-4-[4-(p-tolyl)-6,7-dihydro-4H-thieno[3,2-c]pyridin-5-yl]butane-1,4-dione;
217 1-[4-[(2-fluorophenyl)methyl]-1-piperazinyl]-4-[4-(p-tolyl)-6,7-dihydro-4H-thieno[3,2-c]pyridin-5-yl]butane-1,4-dione;
218 1-(4-methyl-2-phenyl-1-piperazinyl)-4-[4-(p-tolyl)-6,7-dihydro-4H-thieno[3,2-c]-pyridin-5-yl]butane-1,4-dione;
219 1-(4-phenyl-1-piperidinyl)-4-[4-(p-tolyl)-6,7-dihydro-4H-thieno[3,2-c]pyridin-5-yl]butane-1,4-dione;
220 1-[4-(p-tolyl)-6,7-dihydro-4H-thieno[3,2-c]pyridin-5-yl]-4-[2-(2-pyridyl)-1-pyrrolidinyl]butane-1,4-dione;
221 1-[4-(p-tolyl)-6,7-dihydro-4H-thieno[3,2-c]pyridin-5-yl]-4-[3-(3-pyridyl)-1-pyrrolidinyl]butane-1,4-dione;
222 4-oxo-4-[4-(p-tolyl)-6,7-dihydro-4H-thieno[3,2-c]pyridin-5-yl]-N-[2-(2-pyridyl)-ethyl]butanamide;
223 1-[4-[(4-methoxyphenyl)methyl]-1-piperazinyl]-4-[4-(m-tolyl)-6,7-dihydro-4H-thieno[3,2-c]pyridin-5-yl]butane-1,4-dione;
224 1-[4-[(2-fluorophenyl)methyl]-1-piperazinyl]-4-[4-(m-tolyl)-6,7-dihydro-4H-thieno[3,2-c]pyridin-5-yl]butane-1,4-dione;
225 1-(4-methyl-2-phenyl-1-piperazinyl)-4-[4-(m-tolyl)-6,7-dihydro-4H-thieno[3,2-c]-pyridin-5-yl]butane-1,4-dione;
226 1-[4-(m-tolyl)-6,7-dihydro-4H-thieno[3,2-c]pyridin-5-yl]-4-(4-phenyl-1-piperidinyl)butane-1,4-dione;
227 1-[4-(m-tolyl)-6,7-dihydro-4H-thieno[3,2-c]pyridin-5-yl]-4-[2-(2-pyridyl)-1-pyrrolidinyl]butane-1,4-dione;
228 1-[4-(m-tolyl)-6,7-dihydro-4H-thieno[3,2-c]pyridin-5-yl]-4-[3-(3-pyridyl)-1-pyrrolidinyl]butane-1,4-dione;
229 N-methyl-4-[4-(m-tolyl)-6,7-dihydro-4H-thieno[3,2-c]pyridin-5-yl]-4-oxo-N-[2-(4-pyridyl)ethyl]butanamide;
230 1-[4-(3-fluorophenyl)-6,7-dihydro-4H-thieno[3,2-c]pyridin-5-yl]-4-[4-[(4-methoxyphenyl)methyl]-1-piperazinyl]butane-1,4-dione;
231 1-[4-(3-fluorophenyl)-6,7-dihydro-4H-thieno[3,2-c]pyridin-5-yl]-4-[4-[(2-fluoro-phenyl)methyl]-1-piperazinyl]butane-1,4-dione;
232 4-[4-(m-tolyl)-6,7-dihydro-4H-thieno[3,2-c]pyridin-5-yl]-4-oxo-N-(p-tolyl)-butanamide;
233 N-(2,4-dimethylphenyl)-4-[4-(m-tolyl)-6,7-dihydro-4H-thieno[3,2-c]pyridin-5-yl]-4-oxobutanamide;
234 4-[4-(3-fluorophenyl)-6,7-dihydro-4H-thieno[3,2-c]pyridin-5-yl]-N-(1-methyl-6-indazolyl)-4-oxobutanamide;
235 4-[4-(3-fluorophenyl)-6,7-dihydro-4H-thieno[3,2-c]pyridin-5-yl]-4-oxo-N-(p-tolyl)butanamide;
236 4-[4-(3-fluorophenyl)-6,7-dihydro-4H-thieno[3,2-c]pyridin-5-yl]-4-oxo-N-phenyl-butanamide;
237 N-(2-chlorophenyl)-4-[4-(3-fluorophenyl)-6,7-dihydro-4H-thieno[3,2-c]-pyridin-5-yl]-4-oxobutanamide;
238 N-(2,4-dimethylphenyl)-4-[4-(3-fluorophenyl)-6,7-dihydro-4H-thieno[3,2-c]-pyridin-5-yl]-4-oxobutanamide;
239 4-[4-(4-chlorophenyl)-6,7-dihydro-4H-thieno[3,2-c]pyridin-5-yl]-N-(1-methyl-6-indazolyl)-4-oxobutanamide;
240 4-[4-(4-chlorophenyl)-6,7-dihydro-4H-thieno[3,2-c]pyridin-5-yl]-4-oxo-N-(p-tolyl)butanamide;
241 4-[4-(4-chlorophenyl)-6,7-dihydro-4H-thieno[3,2-c]pyridin-5-yl]-N-(4-methoxy-phenyl)-4-oxobutanamide;
242 4-[4-(4-chlorophenyl)-6,7-dihydro-4H-thieno[3,2-c]pyridin-5-yl]-4-oxo-N-phenyl-butanamide;
243 4-[4-(4-chlorophenyl)-6,7-dihydro-4H-thieno[3,2-c]pyridin-5-yl]-N-(2,4-dimethyl-phenyl)-4-oxobutanamide;
244 4-[4-(2,6-dimethylphenyl)-6,7-dihydro-4H-thieno[3,2-c]pyridin-5-yl]-4-oxo-N-[[3-(trifluoromethyl)phenyl]methyl]butanamide;
245 N-(2-cyclohexylethyl)-4-oxo-4-(4-phenyl-6,7-dihydro-4H-thieno[3,2-c]pyridin-5-yl)butanamide;
246 N-(3,3-dimethylbutyl)-4-oxo-4-(4-phenyl-6,7-dihydro-4H-thieno[3,2-c]pyridin-5-yl)butanamide;
247 N-(cyclohexylmethyl)-4-oxo-4-(4-phenyl-6,7-dihydro-4H-thieno[3,2-c]pyridin-5-yl)butanamide;
248 N-[[3-methyl-5-(trifluoromethoxy)phenyl]methyl]-4-oxo-4-(4-phenyl-6,7-dihydro-4H-thieno[3,2-c]pyridin-5-yl)butanamide;
249 N-[[4-methyl-3-(trifluoromethyl)phenyl]methyl]-4-oxo-4-(4-phenyl-6,7-dihydro-4H-thieno[3,2-c]pyridin-5-yl)butanamide;
250 N-[[4-fluoro-3-(trifluoromethyl)phenyl]methyl]-4-oxo-4-(4-phenyl-6,7-dihydro-4H-thieno[3,2-c]pyridin-5-yl)butanamide;
251 4-[4-(2-ethylphenyl)-6,7-dihydro-4H-thieno[3,2-c]pyridin-5-yl]-4-oxo-N-[[3-(trifluoromethyl)phenyl]methyl]butanamide;
252 4-[4-(2-isopropylphenyl)-6,7-dihydro-4H-thieno[3,2-c]pyridin-5-yl]-4-oxo-N-[[3-(trifluoromethyl)phenyl]methyl]butanamide;
253 N-(3-cyclohexylpropyl)-4-oxo-4-(4-phenyl-6,7-dihydro-4H-thieno[3,2-c]pyridin-5-yl)butanamide;
254 4-[4-(3-methyl-2-thienyl)-6,7-dihydro-4H-thieno[3,2-c]pyridin-5-yl]-4-oxo-N-[[3-(trifluoromethyl)phenyl]methyl]butanamide;
255 (1S,2S)-2-[oxo-(4-phenyl-6,7-dihydro-4H-thieno[3,2-c]pyridin-5-yl)methyl]-N-[[3-(trifluoromethyl)phenyl]methyl]-1-cyclopropanecarboxamide;
256 4-[4-(o-tolyl)-6,7-dihydro-4H-thieno[3,2-c]pyridin-5-yl]-4-oxo-N-[[2-(trifluoro-methyl)phenyl]methyl]butanamide;
257 4-[4-(o-tolyl)-6,7-dihydro-4H-thieno[3,2-c]pyridin-5-yl]-4-oxo-N-[[4-methyl)phenyl]methyl]butanamide;
258 4-(7-butyl-5,7-dihydro-4H-thieno[2,3-c]pyridin-6-yl)-4-oxo-N-[[3-(trifluoro-methyl)phenyl]methyl]butanamide;
259 4-[4-(cyclohexylmethyl)-6,7-dihydro-4H-thieno[3,2-c]pyridin-5-yl]-4-oxo-N-[[3-(trifluoromethyl)phenyl]methyl]butanamide;
260 4-(4-cyclopropyl-6,7-dihydro-4H-thieno[3,2-c]pyridin-5-yl)-4-oxo-N-[[3-(trifluoro-methyl)phenyl]methyl]butanamide;
261 4-oxo-4-(4-phenethyl-6,7-dihydro-4H-thieno[3,2-c]pyridin-5-yl)-N-[[3-(trifluoro-methyl)phenyl]methyl]butanamide;
262 4-[4-(4-fluoro-2-methylphenyl)-6,7-dihydro-4H-thieno[3,2-c]pyridin-5-yl]-4-oxo-N-[[3-(trifluoromethyl)phenyl]methyl]butanamide;
263 4-(4-cyclopentyl-6,7-dihydro-4H-thieno[3,2-c]pyridin-5-yl)-4-oxo-N-[[3-(trifluoro-methyl)phenyl]methyl]butanamide;
264 N-[[2-methyl-3-(trifluoromethyl)phenyl]methyl]-4-oxo-4-(4-phenyl-6,7-dihydro-4H-thieno[3,2-c]pyridin-5-yl)butanamide;
265 4-oxo-4-[4-(3-phenylpropyl)-6,7-dihydro-4H-thieno[3,2-c]pyridin-5-yl]-N-[[3-(trifluoromethyl)phenyl]methyl]butanamide;
266 N-[[2-methyl-5-(trifluoromethyl)phenyl]methyl]-4-oxo-4-(4-phenyl-6,7-dihydro-4H-thieno[3,2-c]pyridin-5-yl)butanamide;
267 N-cyclohexyl-4-oxo-4-(4-phenyl-6,7-dihydro-4H-thieno[3,2-c]pyridin-5-yl)-butanamide;
268 N-(2,2-dimethylpropyl)-4-oxo-4-(4-phenyl-6,7-dihydro-4H-thieno[3,2-c]pyridin-5-yl)butanamide;
269 1-(4-phenyl-6,7-dihydro-4H-thieno[3,2-c]pyridin-5-yl)-4-[7-(trifluoromethyl)-3,4-dihydro-1H-isoquinolin-2-yl]butane-1,4-dione;
270 4-oxo-N-pentyl-4-(4-phenyl-6,7-dihydro-4H-thieno[3,2-c]pyridin-5-yl)-butanamide;
271 1-(4-phenyl-6,7-dihydro-4H-thieno[3,2-c]pyridin-5-yl)-4-[5-(trifluoromethyl)-3,4-dihydro-1H-isoquinolin-2-yl]butane-1,4-dione;
272 4-oxo-4-(4-phenyl-6,7-dihydro-4H-thieno[3,2-c]pyridin-5-yl)-N-[5-(trifluoro-methyl)-1-tetralinyl]butanamide;
273 4-oxo-4-(4-phenyl-6,7-dihydro-4H-thieno[3,2-c]pyridin-5-yl)-N-[7-(trifluoro-methyl)-1-tetralinyl]butanamide;
274 4-[7-(o-tolyl)-5,7-dihydro-4H-thieno[2,3-c]pyridin-6-yl]-4-oxo-N-[[3-(trifluoro-methyl)phenyl]methyl]butanamide;
275 4-(7-cyclohexyl-5,7-dihydro-4H-thieno[2,3-c]pyridin-6-yl)-4-oxo-N-[[3-(trifluoro-methyl)phenyl]methyl]butanamide;
and physiologically acceptable salts of any of the foregoing compounds.

12. A pharmaceutical composition comprising a compound as claimed in claim 1, or a compound selected from the group consisting of:
1-morpholino-4-(4-(thiophen-2-yl)-6,7-dihydrothieno[3,2-c]pyridin-5(4H)-yl)butane-1,4-dione,
1-(4-acetylpiperazin-1-yl)-4-(4-(thiophen-2-yl)-6,7-dihydrothieno[3,2-c]pyridin-5(4H)-yl)butane-1,4-dione and
1-(3-phenyl-4,5-dihydropyrazol-1-yl)-4-(4-(thiophen-2-yl)-6,7-dihydrothieno-[3,2-c]pyridin-5(4H)-yl)butane-1,4-dione;
or a physiologically compatible salt thereof and at least one pharmaceutically acceptable carrier or auxiliary substance.

13. A method of treating or inhibiting a disorder or disease state selected from the group consisting of pain, epilepsy, urinary incontinence, anxiety, drug or alcohol dependency, mania, bipolar disorders, migraine, cognitive diseases, dystonia-associated dyskinesias and urinary incontinence in a subject in need thereof, said method comprising administering to said subject a pharmaceutically effective amount of a compound as claimed in claim 1 or a compound selected from the group consisting of:
1-morpholino-4-(4-(thiophen-2-yl)-6,7-dihydrothieno[3,2-c]pyridin-5(4H)-yl)butane-1,4-dione,
1-(4-acetylpiperazin-1-yl)-4-(4-(thiophen-2-yl)-6,7-dihydrothieno[3,2-c]pyridin-5(4H)-yl)butane-1,4-dione and
1-(3-phenyl-4,5-dihydropyrazol-1-yl)-4-(4-(thiophen-2-yl)-6,7-dihydrothieno-[3,2-c]pyridin-5(4H)-yl)butane-1,4-dione;
or a physiologically acceptable salt thereof.
